SEQUENCE LISTING (2 sequences)

SEQ ID NO: 1 - protein, 528 aa, synthetic construct
  source 1..528: note = Human Collagen alpha-1(III) chain
DVKSGVAVGGLAGYPGPAGPPGPPGPPGTSGHPGSPGSPGYQGPPGEPGQAGPSGPPGPPGAIGPSGPAGKDGESGRPGRPGERGLPGPPGIKGPAGIPGFPGMKGHRGFDGRNGEKGETGAPGLKGENGLPGENGAPGPMGPRGAPGERGRPGLPGAAGARGNDGARGSDGQPGPPGPPGTAGFPGSPGAKGEVGPAGSPGSNGAPGQRGEPGPQGHAGAQGPPGPPGINGSPGGKGEMGPAGIPGAPGLMGARGPPGPAGANGAPGLRGGAGEPGKNGAKGEPGPRGERGEAGIPGVPGAKGEDGKDGSPGEPGANGLPGAAGERGAPGFRGPAGPNGIPGEKGPAGERGAPGPAGPRGAAGEPGRDGVPGGPGMRGMPGSPGGPGSDGKPGPPGSQGESGRPGPPGPSGPRGQPGVMGFPGPKGNDGAPGKNGERGGPGGPGPQGPPGKNGETGPQGPPGPTGPGGDKGDTGPPGPQGLQGLPGTGGPPGENGKPGEPGPKGDAGAPGAPGGKGDAGAPGERGPP

SEQ ID NO: 2 - protein, 546 aa, synthetic construct
  source 1..546: note = Collagen Fragment
DVKSGVAVGGLAGYPGPAGPPGPPGPPGTSGHPGSPGSPGYQGPPGEPGQAGPSGPPGPPGAIGPSGPAGKDGESGRPGRPGERGLPGPPGIKGPAGIPGFPGMKGHRGFDGRNGEKGETGAPGLKGENGLPGENGAPGPMGPRGAPGERGRPGLPGAAGARGNDGARGSDGQPGPPGPPGTAGFPGSPGAKGEVGPAGSPGSNGAPGQRGEPGPQGHAGAQGPPGPPGINGSPGGKGEMGPAGIPGAPGLMGARGPPGPAGANGAPGLRGGAGEPGKNGAKGEPGPRGERGEAGIPGVPGAKGEDGKDGSPGEPGANGLPGAAGERGAPGFRGPAGPNGIPGEKGPAGERGAPGPAGPRGAAGEPGRDGVPGGPGMRGMPGSPGGPGSDGKPGPPGSQGESGRPGPPGPSGPRGQPGVMGFPGPKGNDGAPGKNGERGGPGGPGPQGPPGKNGETGPQGPPGPTGPGGDKGDTGPPGPQGLQGLPGTGGPPGENGKPGEPGPKGDAGAPGAPGGKGDAGAPGERGPPAIAGIGGEKAGGFAPYYG

(19) United States
(12) Patent Application Publication
(10) Pub. No.: US 2025/0019540 A1
(43) Pub. Date: Jan. 16, 2025

(54) PROTEIN POLYURETHANE ALLOYS COMPRISING A SUCCINYLATED PROTEIN AND METHODS OF MAKING THE SAME

(71) Applicant: MODERN MEADOW, INC., Nutley, NJ (US)

(72) Inventors: Monique DE LEEUW, Wallington, NJ (US); Gracia EL AYLE, West Orange, NJ (US); Shaobo CAI, Oradell, NJ (US)

(21) Appl. No.: 18/703,611

(22) PCT Filed: Oct. 27, 2022
(86) PCT No.: PCT/US2022/078755

§ 371 Date: Apr. 22, 2024

Related U.S. Application Data
(60) Provisional application No. 63/282,333, filed on Nov. 23, 2021.
(60) Provisional application No. 63/273,658, filed on Oct. 29, 2021.

(51) Int. Cl. C08L 75/04; C07K 14/415

(52) U.S. Cl. CPC C08L 75/04; C07K 14/415

(57) ABSTRACT

Protein polyurethane alloys including one or more succinylated proteins dissolved within one or more polyurethanes. The protein polyurethane alloys may have one or more mechanical properties that are superior to the polyurethane in the absence of protein. The protein polyurethane alloys may be incorporated into a layered material including one or more protein polyurethane alloy layers.

### REFERENCE TO SEQUENCE LISTING SUBMITTED ELECTRONICALLY

The content of the electronically submitted sequence listing ((Name: 4431_083PC02_Seqlisting_ST26.xml; Size: 4,335 bytes; and Date of Creation: Oct. 25, 2022) filed with the application is herein incorporated by reference in its entirety.

### FIELD

This disclosure relates to protein polyurethane alloys comprising one or more succinylated proteins dissolved in a polyurethane. In some embodiments, the protein polymer alloys can have the look, feel, and aesthetic and/or mechanical properties similar to natural leather, and can be used to make goods and articles previously prepared from natural leather.

### BACKGROUND

Leather is a versatile product used across many industries, including the furniture industry, where leather is regularly used as upholstery, the clothing industry, where leather is used to manufacture pants and jackets, the shoe industry, where leather is used to prepare casual and dress shoes, the luggage industry, the handbag and accessory industry, and in the automotive industry. The global trade value for leather is high, and there is a continuing and increasing demand for leather products. However, there are variety of costs, constraints, and social concerns associated with producing natural leather. Foremost, natural leathers are produced from animal skins, and as such, requires raising and slaughtering livestock. Raising livestock requires enormous amounts of feed, pastureland, water, and fossil fuels and contributes to air and waterway pollution, through, for example, greenhouse gases like methane. Leather production also raises social concerns related to the treatment of animals. In recent years, there has also been a fairly well documented decrease in the availability of traditional high quality hides. For at least these reasons, alternative means to meet the demand for leather are desirable.

### BRIEF SUMMARY

The present disclosure provides protein polyurethane alloys suitable for use in a variety of applications, including as a replacement for natural leather.

A first embodiment (1) of the present disclosure is directed to a protein polyurethane alloy comprising a succinylated protein dissolved within a polyurethane.

In a second embodiment (2), the succinylated protein of the protein polyurethane alloy of the first embodiment (1) is a succinylated soy protein.

In a third embodiment (3), the succinylated protein of the protein polyurethane alloy according to the second embodiment (2) is succinylated soy protein isolate.

In a fourth embodiment (4), the protein polyurethane alloy according to the second embodiment (2) or the third embodiment (3) has a second DMA modulus transition onset temperature ranging from about 130° C. to about 200° C.

In a fifth embodiment (5), the protein polyurethane alloy according to any one of embodiments (2)-(4) has a Young's modulus ranging from about 80 MPa to about 155 MPa.

In a sixth embodiment (6), the polyurethane of the protein polyurethane alloy according to any one of embodiments (2)-(5) has a second DMA modulus transition onset temperature in the absence of soy protein, and the protein polyurethane alloy has a second DMA modulus transition onset temperature ranging from about 15° C. to about 100° C. greater than the second DMA modulus transition onset temperature of the polyurethane in the absence of soy protein.

In a seventh embodiment (7), the protein polyurethane alloy according to any one of embodiments (2)-(6) has a tensile strength ranging from about 10 MPa to about 18 MPa.

In an eighth embodiment (8), the polyurethane of the protein polyurethane alloy according to any one of embodiments (2)-(7) has a moisture vapor transmission rate in the absence of soy protein, and the protein polyurethane alloy has a moisture vapor transmission rate ranging from about 200 g/m²/24 hr to about 300 g/m²/24 hr greater than the moisture vapor transmission rate of the polyurethane in the absence of soy protein.

In a ninth embodiment (9) the protein polyurethane alloy according to any one of embodiments (1)-(8) comprises about 10 wt % to about 50 wt % of the succinylated protein and about 50 wt % to about 90 wt % of the polyurethane.

In a tenth embodiment (10), the protein polyurethane alloy according to any one of embodiments (1)-(8) comprises about 20 wt % to about 35 wt % of the succinylated protein and about 65 wt % to about 80 wt % of the polyurethane.

In an eleventh embodiment (11), the protein polyurethane alloy according to any one of embodiments (2)-(10) has a color stability defined by a change in a spectrometer measured b* value in the CIELab color space of 6 or less, wherein the change in the spectrometer measured b* value is a change in b* over six days with the protein polyurethane alloy placed in a hydrolysis chamber at 70° C. and 95% humidity.

In a twelfth embodiment (12), the succinylated protein of the protein polyurethane alloy according to any one of embodiments (2)-(11) has a lysine modification of about 20% to 100%.

In a thirteenth embodiment (13), the succinylated protein of the protein polyurethane alloy according to any one of embodiments (2)-(12) has a solubility of about 50% to 100%.

In a fourteenth embodiment (14), the protein polyurethane alloy according to any one of embodiments (1)-(13) further comprises a colored dye.

A fifteenth embodiment (15) is directed to a succinylated soy protein comprising an average lysine modification of about 20% to 100% and a solubility of about 50% to 100%.

In a sixteenth embodiment (16), the succinylated soy protein according to the fifteenth embodiment (15) is succinylated soy protein isolate.

A seventeenth embodiment (17) is directed to a method of making a protein polyurethane alloy, the method comprising succinylating a protein; blending the succinylated protein with one or more polyurethanes in an aqueous solution to form a blended mixture comprising the succinylated protein dissolved within the one or more polyurethanes; and removing solvent from the aqueous solution.

In an eighteenth embodiment (18), succinylating the protein according to the seventeenth embodiment (17) comprises adding succinic anhydride to a protein aqueous solution comprising the protein; purifying the protein aqueous solution; and drying the purified protein aqueous solution.

In a nineteenth embodiment (19), the protein in the method according to the seventeenth embodiment (17) or the eighteenth embodiment (18) comprises soy protein.

In a twentieth embodiment (20), the protein in the method according to the seventeenth embodiment (17) or the eighteenth embodiment (18) comprises soy protein isolate.

### BRIEF DESCRIPTION OF THE FIGURES

The accompanying figures, which are incorporated herein, form part of the specification and illustrate embodiments of the present disclosure. Together with the description, the figures further serve to explain the principles of and to enable a person skilled in the relevant art(s) to make and use the disclosed embodiments. These figures are intended to be illustrative, not limiting. Although the disclosure is generally described in the context of these embodiments, it should be understood that it is not intended to limit the scope of the disclosure to these particular embodiments. In the drawings, like reference numbers indicate identical or functionally similar elements.

FIG. 1 is a graph depicting percent solubility of succinylated soy protein isolates versus the succinic anhydride concentration for the succinylation reaction used to succinylate the soy protein isolates.

FIG. 2 is a graph depicting percent lysine succinylation of succinylated soy protein isolates versus the succinic anhydride concentration for the succinylation reaction used to succinylate the soy protein isolates.

FIG. 3 is a graph comparing the Young's modulus of a soy protein polyurethane alloy made with non-succinylated soy protein isolate and soy protein polyurethane alloys made with soy protein isolates succinylated at different succinic anhydride concentrations according to some embodiments.

FIG. 4 is a graph comparing the tensile strength of a soy protein polyurethane alloy made with non-succinylated soy protein isolate and soy protein polyurethane alloys made with soy protein isolates succinylated at different succinic anhydride concentrations according to some embodiments.

FIG. 5 is a graph comparing the elongation length of a soy protein polyurethane alloy made with non-succinylated soy protein isolate and soy protein polyurethane alloys made with soy protein isolates succinylated at different succinic anhydride concentrations according to some embodiments.

FIG. 6 is a DMA thermogram comparing a polyurethane sample made of 100% L3360 and four succinylated soy protein alloys comprising L3360 according to some embodiments.

FIG. 7 is a graph comparing complex viscosity of a non-succinylated soy protein isolate solution and soy protein isolate solutions succinylated at different succinic anhydride concentrations according to some embodiments.

FIG. 8 is a graph comparing loss and storage moduli of a non-succinylated soy protein isolate solution and soy protein isolate solutions succinylated at different succinic anhydride concentrations according to some embodiments.

FIGS. 9A and 9B are graphs comparing the complex viscosity of a non-succinylated soy protein polyurethane alloy solution and soy protein polyurethane alloy solutions made with soy protein isolate succinylated at different succinic anhydride concentrations according to some embodiments.

FIG. 10 is a graph comparing the storage and loss moduli of a non-succinylated soy protein polyurethane alloy solution and soy protein polyurethane alloy solutions made with soy protein isolate succinylated at different succinic anhydride concentrations according to some embodiments.

FIG. 11 is a graph comparing the color stability for a soy protein polyurethane alloy made with non-succinylated soy protein isolate and soy protein polyurethane alloys made with soy protein isolates succinylated at different succinic anhydride concentrations according to some embodiments.

FIG. 12 is a graph comparing the Young's modulus of a soy protein polyurethane alloy made with non-succinylated soy protein isolate and soy protein polyurethane alloys made with soy protein isolates succinylated at different succinic anhydride concentrations and pHs according to some embodiments.

FIG. 13 is a graph comparing the tensile strength of a soy protein polyurethane alloy made with non-succinylated soy protein isolate and soy protein polyurethane alloys made with soy protein isolates succinylated at different succinic anhydride concentrations and pHs according to some embodiments.

FIG. 14 is a graph comparing the elongation length of a soy protein polyurethane alloy made with non-succinylated soy protein isolate and soy protein polyurethane alloys made with soy protein isolates succinylated at different succinic anhydride concentrations and pHs according to some embodiments.

FIG. 15 is a graph comparing the complex viscosity of a non-succinylated soy protein polyurethane alloy solution and soy protein polyurethane alloy solutions made with soy protein isolate succinylated at different succinic anhydride concentrations and pHs according to some embodiments.

FIG. 16 is a bar graph comparing the color stability of a soy protein polyurethane alloy made with non-succinylated soy protein isolate and soy protein polyurethane alloys made with soy protein isolates succinylated at different succinic anhydride concentrations and pHs according to some embodiments.

FIG. 17A is a graph comparing the measured b* values for a turquoise dyed soy protein polyurethane made with non-succinylated soy protein isolate and a turquoise dyed soy protein polyurethane alloy made with succinylated soy protein isolate according to some embodiments.

FIG. 17B is a graph comparing the measured a* values for a Bordeaux dyed soy protein polyurethane alloy made with non-succinylated soy protein isolate and a Bordeaux dyed soy protein polyurethane alloy made with succinylated soy protein isolate according to some embodiments.

FIG. 18 is a bar graph comparing the moisture vapor transmission rate of a soy protein polyurethane alloy made with non-succinylated soy protein isolate and a soy protein polyurethane alloy made with succinylated soy protein isolate according to some embodiments.

FIG. 19 is a graph comparing the Young's modulus a soy protein polyurethane alloy made with non-succinylated soy protein isolate and a soy protein polyurethane alloy made with succinylated soy protein isolate according to some embodiments.

FIG. 20 is a graph comparing the tensile strength of a soy protein polyurethane alloy made with non-succinylated soy protein isolate and a soy protein polyurethane alloy made with succinylated soy protein isolate according to some embodiments.

FIG. 21 is a graph comparing the elongation length of a soy protein polyurethane alloy made with non-succinylated soy protein isolate and a soy protein polyurethane alloy made with succinylated soy protein isolate according to some embodiments.

FIG. 22 is a DMA thermogram comparing a polyurethane sample made of 100% RC-2214, a non-succinylated soy protein alloy comprising RC-2214, and a succinylated soy protein alloy comprising RC-2214 according to some embodiments.

FIG. 23 is a representative DMA graph illustrating the methodology of measuring first and second DMA modulus transition onset temperatures.

FIG. 24 illustrates a layered material according to some embodiments.

FIG. 25 illustrates a layered material according to some embodiments.

FIG. 26 is a block diagram illustrating a method for making a layered material according to some embodiments.

FIGS. 27A-27F illustrate a method of making a layered material according to some embodiments.

FIG. 28 illustrates a spacer fabric according to some embodiments.

### DETAILED DESCRIPTION

The indefinite articles “a,” “an,” and “the” include plural referents unless clearly contradicted or the context clearly dictates otherwise.

The term “comprising” is an open-ended transitional phrase. A list of elements following the transitional phrase “comprising” is a non-exclusive list, such that elements in addition to those specifically recited in the list can also be present. The phrase “consisting essentially of” limits the composition of a component to the specified materials and those that do not materially affect the basic and novel characteristic(s) of the component. The phrase “consisting of” limits the composition of a component to the specified materials and excludes any material not specified.

Where a range of numerical values comprising upper and lower values is recited herein, unless otherwise stated in specific circumstances, the range is intended to include the endpoints thereof, and all integers and fractions within the range. It is not intended that the disclosure or claims be limited to the specific values recited when defining a range. Further, when an amount, concentration, or other value or parameter is given as a range, one or more ranges, or as list of upper values and lower values, this is to be understood as specifically disclosing all ranges formed from any pair of any upper range limit or value and any lower range limit or value, regardless of whether such pairs are separately disclosed. Finally, when the term “about” is used in describing a value or an end-point of a range, the disclosure should be understood to include the specific value or end-point referred to. Whether or not a numerical value or end-point of a range recites “about,” the numerical value or end-point of a range is intended to include two embodiments: one modified by “about,” and one not modified by “about.”

As used herein, the term “about” refers to a value that is within +10% of the value stated. For example, about 3 MPa can include any number between 2.7 MPa and 3.3 MPa. As used herein, a first layer described as “attached to” a second layer means that the layers are attached to each other either by direct contact and attachment between the two layers or via one or more intermediate adhesive layers. An intermediate adhesive layer can be any layer that serves to attach a first layer to a second layer.

As used herein, the phrase “disposed on” means that a first component (e.g., layer) is in direct contact with a second component. A first component “disposed on” a second component can be deposited, formed, placed, or otherwise applied directly onto the second component. In other words, if a first component is disposed on a second component, there are no components between the first component and the second component.

As used herein, the phrase “disposed over” means other components (e.g., layers or substrates) may or may not be present between a first component and a second component.

As used herein, a “bio-based polyurethane” is a polyurethane where the building blocks of polyols, such as diols and diacids like succinic acid, are derived from a biological material such as corn starch.

As used herein, the term “substantially free of” means that a component is present in a detectable amount not exceeding about 0.1 wt %.

As used herein, the term “free of” means that a component is not present in a blend or material (e.g., a protein polyurethane alloy), even in trace amounts.

As used herein “collagen” refers to the family of at least 28 distinct naturally occurring collagen types including, but not limited to collagen types I, II, III, IV, V, VI, VII, VIII, IX, X, XI, XII, XIII, XIV, XV, XVI, XVII, XVIII, XIX, and XX. The term collagen as used herein also refers to collagen prepared using recombinant techniques. The term collagen includes collagen, collagen fragments, collagen-like proteins, triple helical collagen, alpha chains, monomers, gelatin, trimers and combinations thereof. Recombinant expression of collagen and collagen-like proteins is known in the art (see, e.g., Bell, EP 1232182B1, Bovine collagen and method for producing recombinant gelatin; Olsen, et al., U.S. Pat. No. 6,428,978 and VanHeerde, et al., U.S. Pat. No. 8,188,230, incorporated by reference herein in their entireties) Unless otherwise specified, collagen of any type, whether naturally occurring or prepared using recombinant techniques, can be used in any of the embodiments described herein. That said, in some embodiments, the collagen described herein can be prepared using bovine Type I collagen. Collagens are characterized by a repeating triplet of amino acids, -(Gly-X-Y)n-, so that approximately one-third of the amino acid residues in collagen are glycine. X is often proline and Y is often hydroxyproline. Thus, the structure of collagen may consist of three intertwined peptide chains of differing lengths. Different animals may produce different amino acid compositions of the collagen, which may result in different properties (and differences in the resulting leather).

Any type of collagen, truncated collagen, unmodified or post-translationally modified, or amino acid sequence-modified collagen can be used as part of the protein polyurethane alloy.

In some embodiments, the collagen can be plant-based collagen. For example, the collagen can be a plant-based collagen made by CollPlant.

In some embodiments, a collagen solution can be fibrillated into collagen fibrils. As used herein, collagen fibrils refer to nanofibers composed of tropocollagen or tropocollagen-like structures (which have a triple helical structure). In some embodiments, triple helical collagen can be fibrillated to form nanofibrils of collagen.

In some embodiments, a recombinant collagen can comprise a collagen fragment of the amino acid sequence of a native collagen molecule capable of forming tropocollagen (trimeric collagen). A recombinant collagen can also comprise a modified collagen or truncated collagen having an amino acid sequence at least 70, 80, 90, 95, 96, 97, 98, or 99% identical or similar to a native collagen amino acid sequence (or to a fibril forming region thereof or to a segment substantially comprising [Gly-X-Y]n). In some embodiments, the collagen fragment can be a 50 kDa portion of a native collagen. Native collagen sequences include the amino acid sequences of Col1A1, Col1A2, and Col3A1, described by Accession Nos. NP_001029211.1, NP_776945.1 and NP_001070299.1, which are incorporated by reference. In some embodiments, the collagen fragment can be a portion of human collagen alpha-1 (III) (Col3A1; Uniprot #P02461, Entrez Gene ID #1281). In some embodiments, the collagen fragment can comprise the amino acid sequence listed as SEQ ID NO: 1. In some embodiments, the collagen fragment can comprise the amino acid sequence listed as SEQ ID NO: 2.

Methods of producing recombinant collagen and recombinant collagen fragments are known in the art. For example, U.S. Pub. Nos. 2019/0002893, 2019/0040400, 2019/0093116, and 2019/0092838 provide methods for producing collagen and collagen fragments that can be used to produce the recombinant collagen and recombinant collagen fragments disclosed herein. The contents of these four publications are incorporated by reference in their entirety.

Protein polyurethane alloys described herein can comprise a succinylated protein that is miscible with only one of a plurality of phases of a polyurethane, or a plurality of polyurethanes, with which it is blended. For example, in some embodiments, the protein polyurethane alloy can include a succinylated protein that is miscible with only the hard phase of the polyurethane, or the plurality of polyurethanes, having both a hard phase and a soft phase. Protein polyurethane alloys described herein can be free of or substantially free of protein in form of particles dispersed in a polyurethane. For example, in some embodiments, the protein polyurethane alloys can be free of or substantially free of protein particles having an average diameter of greater than 1 micron (μm).

In some embodiments, the protein polyurethane alloys can be free of or substantially free of soy protein particles having an average diameter of greater than 1 micron (μm). In some embodiments, the protein polyurethane alloys can be free of or substantially free of collagen particles having an average diameter of greater than 1 micron (μm). In some embodiments, the protein polyurethane alloys can be free of or substantially free of gelatin particles having an average diameter of greater than 1 micron (μm). In some embodiments, the protein polyurethane alloys can be free of or substantially free of bovine serum albumin particles having an average diameter of greater than 1 micron (μm). In some embodiments, the protein polyurethane alloys can be free of or substantially free of pea protein particles having an average diameter of greater than 1 micron (μm). In some embodiments, the protein polyurethane alloys can be free of or substantially free of egg white albumin particles having an average diameter of greater than 1 micron (μm). In some embodiments, the protein polyurethane alloys can be free of or substantially free of casein protein particles having an average diameter of greater than 1 micron (μm). In some embodiments, the protein polyurethane alloys can be free of or substantially free of peanut protein particles having an average diameter of greater than 1 micron (μm). In some embodiments, the protein polyurethane alloys can be free of or substantially free of edestin protein particles having an average diameter of greater than 1 micron (μm). In some embodiments, the protein polyurethane alloys can be free of or substantially free of whey protein particles having an average diameter of greater than 1 micron (μm). In some embodiments, the protein polyurethane alloys can be free of or substantially free of karanja protein particles having an average diameter of greater than 1 micron (μm). In some embodiments, the protein polyurethane alloys can be free of, or substantially free of, cellulase particles having an average diameter of greater than 1 micron (μm). In some embodiments, the protein polyurethane alloys can be free of, or substantially free of, recombinant collagen fragment particles having an average diameter of greater than 1 micron (μm). In some embodiments, the protein polyurethane alloys can be free of, or substantially free of, hemp protein particles having an average diameter of greater than 1 micron (μm).

The present disclosure provides a unique combination of a succinylated protein and a polyurethane in which the succinylated protein is dissolved within the polyurethane In particular embodiments, the present disclosure provides a unique combination of a succinylated protein and a polyurethane in which the succinylated protein is dissolved in only the hard phase of the polyurethane. The present disclosure also provides methods of making succinylated proteins and the protein polyurethane alloys described herein. The present disclosure also provides layered materials including one or more of the protein polyurethane alloy layers and methods of making the layered materials. The protein polyurethane alloys and the protein polyurethane alloy layers can include one or more types of succinylated protein and one or more polyurethanes.

Proteins for use in the polyurethane alloys disclosed herein are succinylated proteins. A succinylated protein is a protein modified with the addition of a succinyl group to a side chain of an amino acid in the protein. Most commonly, the succinyl group is added to lysine side chains. The method of adding a succinyl group to a side chain of an amino acid in a protein is commonly referred to as protein succinylation. Addition of succinyl groups on a protein can alter the protein's functional and structural properties. In some cases, the addition of a relatively large modification like a succinyl moiety can be expected to alter the tertiary structure of the protein. Further, with the addition of a succinyl moiety, a lysine side chain can be altered from a primary amine to an acid, making it more hydrophilic, and can change its charge from positive to negative in physiological pH. Succinylation can be accomplished using techniques and chemistry well known the art.

A succinylated protein for use in the polyurethane alloys disclosed herein can be a protein containing lysine. In some embodiments, the succinylated protein can be a succinylated soy protein. In some embodiments, the succinylated protein can be a succinylated soy protein isolate. In some embodiments, the succinylated protein can be a succinylated collagen. In some embodiments, the succinylated protein can be a succinylated gelatin. In some embodiments, the succinylated protein can be succinylated bovine serum albumin. In some embodiments, the succinylated protein can be a succinylated pea protein. In some embodiments, the succinylated protein can be succinylated egg white albumin. In some embodiments, the succinylated protein can be a succinylated casein protein. In some embodiments, the succinylated protein can be a succinylated peanut protein. In some embodiments, the succinylated protein can be a succinylated edestin protein. In some embodiments, the succinylated protein can be a succinylated whey protein. In some embodiments, the succinylated protein can be a succinylated karanja protein. In some embodiments, the succinylated protein can be a succinylated cellulase. In some embodiments, the succinylated protein can be a succinylated hemp protein.

In some embodiments, succinylation as described herein can increase the solubility of a protein in water. In some embodiments, succinylation can increase the average lysine modification of a protein. In some embodiments, succinylation can increase the solubility of a protein in water and increase the average lysine modification of a protein.

By achieving a suitable level of succinylation, and without wishing to be bound by theory, it is believed characteristics imparted by succinylation can enhance the interaction of the succinylated protein with the hard phase of a polyurethane. And by increasing the interaction with the hard phase, it is believed the dissolution of the succinylated protein within the hard phase can be enhanced. In some embodiments, the level of succinylation for a succinylated protein can be quantified by the solubility of the succinylated protein in water, the average lysine modification of the succinylated protein, or both.

In some embodiments, the succinylated protein can have a solubility in water, measured as described below, of about 50% to about 100%, including subranges. For example, in some embodiments, the succinylated protein can have a solubility in water of about 50% to about 100%, about 51% to about 100%, about 52% to about 100%, about 53% to about 100%, about 54% to about 100%, about 55% to about 100%, about 56% to about 100%, about 57% to about 100%, about 58% to about 100%, about 59% to about 100%, about 60% to about 100%, about 61% to about 100%, about 62% to about 100%, about 63% to about 100%, about 64% to about 100%, about 65% to about 100%, about 66% to about 100%, about 67% to about 100%, about 68% to about 100%, about 69% to about 100%, about 70% to about 100%, about 71% to about 100%, about 72% to about 100%, about 73% to about 100%, about 74% to about 100%, about 75% to about 100%, about 76% to about 100%, about 77% to about 100%, about 78% to about 100%, about 79% to about 100%, about 80% to about 100%, about 81% to about 100%, about 82% to about 100%, about 83% to about 100%, about 84% to about 100%, about 85% to about 100%, about 86% to about 100%, about 87% to about 100%, about 88% to about 100%, about 89% to about 100%, about 90% to about 100%, about 91% to about 100%, about 92% to about 100%, about 93% to about 100%, about 94% to about 100%, or about 95% to about 100%, or within a range having any two of these values as endpoints, inclusive of the endpoints.

In some embodiments, the succinylated protein can have a solubility in water of about 60% to about 90%, 61% to about 90%, about 62% to about 90%, about 63% to about 90%, about 64% to about 90%, about 65% to about 90%, about 66% to about 90%, about 67% to about 90%, about 68% to about 90%, about 69% to about 90%, about 70% to about 90%, about 71% to about 90%, about 72% to about 90%, about 73% to about 90%, about 74% to about 90%, about 75% to about 90%, about 76% to about 90%, about 77% to about 90%, about 78% to about 90%, about 79% to about 90%, about 80% to about 90%, about 81% to about 90%, about 82% to about 90%, about 83% to about 90%, about 84% to about 90%, or about 85% to about 90%. In still further embodiments, the succinylated protein can have a solubility in water of about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%.

Unless otherwise specified, the solubility of a protein in water is measured according to the following method. The succinylated protein is suspended to a 5% (w/v) aqueous solution with DI water and the total solids are measured with a moisture analyzer. Then 35 mL of the 5% solution is centrifuged in a 50 mL tube for 10 minutes at 15,000×g (times gravity). After centrifugation, the supernatant is decanted and the volume and total solids of the supernatant (soluble fraction) is measured. The solubility is then calculated as follows: solubility=(total solids of the supernatant*volume of the supernatant)/(Total solids of the 5% starting solution*volume of the 5% starting solution).

In some embodiments, the succinylated protein can have an average lysine modification, measured as specified below, of about 20% to about 100%, including subranges. For example, in some embodiments, the succinylated protein can have an average lysine modification of about 20% to about 100%, about 22% to about 100%, about 24% to about 100%, about 25% to about 100%, about 26% to about 100%, about 28% to about 100%, about 30% to about 100%, about 32% to about 100%, about 34% to about 100%, about 35% to about 100%, about 36% to about 100%, about 38% to about 100%, about 40% to about 100%, about 42% to about 100%, about 44% to about 100%, about 45% to about 100%, about 46% to about 100%, about 48% to about 100%, about 50% to about 100%, about 52% to about 100%, about 54% to about 100%, about 55% to about 100%, about 56% to about 100%, about 58% to about 100%, about 60% to about 100%, about 62% to about 100%, about 64% to about 100%, about 65% to about 100%, about 66% to about 100%, about 68% to about 100%, about 70% to about 100%, about 72% to about 100%, about 74% to about 100%, about 75% to about 100%, about 76% to about 100%, about 78% to about 100%, about 80% to about 100%, about 82% to about 100%, about 84% to about 100%, about 85% to about 100%, about 86% to about 100%, about 88% to about 100%, about 90% to about 100%, about 92% to about 100%, about 94% to about 100%, or about 95% to about 100%, or within a range having any two of these values as endpoints, inclusive of the endpoints.

In some embodiments, the succinylated protein can have an average lysine modification of about 60% to about 90%, about 62% to about 90%, about 64% to about 90%, about 65% to about 90%, about 66% to about 90%, about 68% to about 90%, about 70% to about 90%, about 72% to about 90%, about 74% to about 90%, about 75% to about 90%, about 76% to about 90%, about 78% to about 90%, about 80% to about 90%, about 82% to about 90%, about 84% to about 90%, or about 85% to about 90%.

In some embodiments, the succinylated protein can have an average lysine modification of about 60% to about 80%, about 62% to about 80%, about 64% to about 80%, about 65% to about 80%, about 66% to about 80%, about 68% to about 80%, about 70% to about 80%, about 72% to about 80%, about 74% to about 80%, or about 75% to about 80%.

In still further embodiments, the succinylated protein can have an average lysine modification of about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%.

Unless otherwise specified, the average lysine modification for a succinylated protein disclosed herein is measured using the following lysine modification assay. A protein sample is digested using trypsin and loaded onto a liquid chromatography-mass spectrometer (LC/MS). After running the sample on the LC/MS, the sample is mapped by matching the digested peptides against the reference protein sequence for Beta-conglycinin alpha′ subunit (Gene CG-1; Organism: Glycine max (soybean) (Glycine hispida), which contains the possibility for both modified and unmodified lysine residues. Following peptide mapping, peak areas of matched peptides were used to calculate the proportion of modified to unmodified lysine at each lysine residue in the protein sequence and then the average % lysine modification was calculated based on all detected lysines. The following calculations are used to calculate the average % lysine modification. For each detected lysine, the lysine modification % was calculated as equal to the (SUM succinylated lysine/(SUM succinylated lysine+SUM non-succinylated lysine). The overall average % lysine modification is then calculated by averaging the individual lysine modification % of all the detected lysines.

Suitable polyurethanes for use in the protein polyurethane alloys described herein include those that comprise at least two phases including a “soft phase” and a “hard phase.” The soft phase is formed from polyol segments within the polyurethane that separate from the urethane-containing phase due to differences in polarity. The urethane-containing phase is referred to as the hard phase. This phase separation is well known in the art and is the basis of the many of the properties of polyurethanes.

The soft phase is typically elastomeric at room temperature, and typically has a softening point or glass transition temperature (Tg) below room temperature. The Tg can be measured by Dynamic Mechanical Analysis (DMA) and quantified by either the peak of tan (8) or the onset of the drop in storage modulus. Alternately, Tg can be measured by Differential Scanning calorimetry (DSC). In some cases, there can be crystallinity in the soft phase, which can be seen as a melting point, typically between 0° C. and about 60° C.

The hard phase typically has a Tg or melting point above room temperature, more typically above about 80° C. The softening of the hard phase can be measured by measuring the onset of the drop in storage modulus (sometimes referred to as stiffness) as measured by DMA.

The “soft phase” for the polyurethane or the protein polyurethane alloy including the polyurethane comprises the polyol component of the polyurethane. Its function is to be soft and flexible at temperatures above its Tg to lend toughness, elongation, and flexibility to the polyurethane. Typical soft segments can comprise polyether polyols, polyester polyols, polycarbonate polyols, and mixtures thereof. They typically range in molecular weight from about 250 daltons to greater than about 5 kiloDaltons. The “hard phase” for the polyurethane or the protein polyurethane alloy including the polyurethane comprises the urethane segments of the polymer that are imparted by the isocyanate(s) used to connect the polyols along with short chain diols such as butane diol, propane diol, and the like. Typical isocyanates useful for the present polyurethanes include, but are not limited to, hexamethylene diisocyanate, isophorone diisocyanate, methylene diisocyanate, phenyl diisocyanate, and the like. These molecules are more polar and stiffer than the polyols used to make the soft segment. Therefore, the hard segment is stiffer and has a higher softening point compared to the soft segment. The function of the hard phase is to provide, among other properties, strength, temperature resistance, and abrasion resistance to the polyurethane.

In some embodiments described herein, the succinylated protein can be miscible with only the hard phase, leaving soft phase transitions substantially unaltered. Without wishing to be bound particular theory, it is believed that when the protein is dissolved in the hard phase, it significantly increases the temperature at which the hard phase begins to soften, thus increasing the temperature resistance of the alloy described herein. Protein polyurethane alloys described herein can also have increased stiffness and increased strength relative to the base polyurethane (i.e., the polyurethane by itself, in the absence of protein).

Protein polyurethane alloys and layers described herein can be formed by blending one or more succinylated proteins with one or more water-borne polyurethane dispersions in a liquid state and drying the blend. In some embodiments, the protein polyurethane alloys and layers described herein can be formed by blending one or more succinylated proteins dissolved or dispersed in an aqueous solution with one or more water-borne polyurethane dispersions in a liquid state and drying the blend. In some embodiments, the polyurethane dispersion can be ionic, and either anionic or cationic. In some embodiments, the polyurethane dispersion can be nonionic. In some embodiments, the blended protein and polyurethane can be formed into a sheet and can, in certain embodiments, be attached to a substrate layer using a suitable attachment process, such as direct coating, a lamination process or a thermo-molding process. In certain embodiments, the lamination process can include attaching the sheet to the substrate layer using an adhesive layer. In some embodiments, the blended protein and polyurethane can be coated or otherwise deposited over a substrate layer to attach the blended protein and polyurethane to the substrate layer. In some embodiments, attaching the blended succinylated protein and polyurethane to the substrate layer can result in a portion of the blended protein and polyurethane being integrated into a portion of the substrate layer.

In a protein polyurethane alloy including one or more miscible succinylated proteins and polyurethanes, the one or more succinylated proteins can be dissolved within the hard phase of the one or more polyurethanes. The protein polyurethane alloy can include at least one succinylated protein miscible with the hard phase of one or more polyurethanes in the alloy. In some embodiments, the protein polyurethane alloy can include a plurality of succinylated proteins and/or a plurality of polyurethane hard phases that are miscible with each other. In all of these embodiments, and without wishing to be bound by a particular theory, the succinylated protein, or plurality of proteins, is believed to be dissolved in the hard phase of the polyurethane, or plurality of polyurethanes.

One or more succinylated proteins dissolved within the hard phase of one or more polyurethanes can form a homogenous mixture when blended. In some embodiments, the protein polyurethane alloy can include a plurality of succinylated proteins dissolved within or more polyurethanes such that the succinylated proteins and the polyurethane(s) form a homogenous mixture when blended and dried. Typically, the protein polyurethane alloy including a homogenous mixture of succinylated protein and polyurethane does not include a substantial amount of protein not dissolved in the polyurethane. That said, and in some embodiments, the protein polyurethane alloy can include a fraction of succinylated protein dispersed within the polyurethane.

In embodiments described herein, the miscibility of the succinylated protein with the hard phase of the polyurethane can increase the DMA modulus transition softening onset temperature of the hard phase in a protein polyurethane alloy without significantly changing one or more other thermo-mechanical properties of the alloy relative to the thermo-mechanical properties of the polyurethane by itself. For example, the miscibility of the succinylated protein with the hard phase of the polyurethane can increase the DMA modulus transition onset temperature of the hard phase in the protein polyurethane alloy without significantly changing the DMA transition temperature of the soft phase in the alloy relative to the DMA transition temperature of the soft phase of the polyurethane by itself.

The DMA transition temperature of the soft phase can be referred to as the glass transition temperature (Tg) of a polyurethane or the protein polyurethane alloy. The DMA transition temperature of the soft phase, or Tg, can be quantified as (i) the DMA storage modulus transition onset temperature of the soft phase (referred to herein as the “first DMA modulus transition onset temperature”) or (ii) the DMA tan (8) peak temperature corresponding to the soft phase. The DMA transition temperature of the hard phase can be measured by the onset of the drop in the storage modulus of the polyurethane or the polyurethane protein alloy and can be quantified as the DMA modulus transition onset temperature of the hard phase (referred to herein as the “second DMA modulus transition onset temperature”). In some embodiments, the second DMA modulus transition onset temperature of the protein polyurethane alloy can be above about 130° C.

Although many types of succinylated proteins are contemplated for use in the protein polyurethane alloys described herein including, for example, succinylated collagen and soy proteins, it is understood that for all of the embodiments disclosed herein, the protein can be a protein other than collagen and/or a protein other than a soy protein. Thus, in some embodiments, the succinylated protein dissolved in the protein polyurethane alloy can be a protein other than collagen. In other embodiments, the succinylated protein dissolved in the protein polyurethane alloy can be a protein other than a soy protein. In some embodiments, the succinylated protein dissolved in the protein polyurethane alloy can be a protein other than collagen and a protein other than a soy protein. In some embodiments, the protein polyurethane alloy can be free of or substantially free of collagen. In some embodiments, the protein polyurethane alloy can be free of or substantially free of soy protein. In some embodiments, the protein polyurethane alloy can be free of or substantially free of soy protein and collagen.

As previously discussed, the soft phase and the hard phase of the polyurethane can be measured using Dynamic Mechanical Analysis (DMA). Accordingly, the one or more polyurethanes included in the protein polyurethane alloys described herein can have at least two DMA transition temperatures, one corresponding to the soft phase and one corresponding to the hard phase. The DMA transition temperature of the soft phase can be quantified as a “first DMA modulus transition onset temperature” or DMA tan (8) peak temperature corresponding to the soft phase. The DMA transition temperature of the hard phase can be quantified by a “second DMA modulus transition onset temperature.” The first DMA modulus transition onset temperature or a DMA tan (8) peak temperature is a lower DMA transition temperature and the second DMA modulus transition onset temperature is a higher DMA transition temperature.

Similarly, the protein polyurethane alloys described herein can have at least two phases. The at least two phases can include the soft phase and the hard phase. Different phases of the alloys can be measured and quantified in the same manner as described above for the polyurethanes.

The polyurethane or the protein polyurethane alloy having first and second DMA transition temperatures means that it has a first DMA transition temperature that occurs at a lower temperature than the second DMA transition temperature. However, the first and second transition temperatures need not be sequential transition temperatures. Other DMA transition temperatures could occur between the first and second transitions.

In some embodiments, the first DMA modulus transition onset temperature for a polyurethane can be below 30° C. In some embodiments, the first DMA modulus transition onset temperature for a polyurethane can range from about −65° C. to about 30° C., including subranges. For example, in some embodiments, the first DMA modulus transition onset temperature for a polyurethane can be about −65° C., about −60° C., about −55° C., about −50° C., about −45° C., about −40° C., about −35° C., about −30° C., about −25° C., about −20° C., about −15° C., about −10° C., about −5° C., about −1° C., 0° C., about 1° C., about 5° C., about 10° C., about 15° C., about 20° C., about 25° C., or about 30° C., or within a range having any two of these values as endpoints, inclusive of the endpoints. In some embodiments, the first DMA modulus transition onset temperature of a polyurethane can be about −65° C. to about 30° C., about −65° C. to about 25° C., about −65° C. to about 20° C., about −65° C. to about 15° C., about −65° C. to about 10° C., about −65° C. to about 5° C., about −65° C. to about 1° C., about −65° C. to 0° C., about −65° C. to about −1° C., about −65° C. to about −5° C., about −65° C. to about −10° C., about −65° C. to about −15° C., about −65° C. to about −20° C., about −65° C. to about −25° C., about −65° C. to about −30° C., about −65° C. to about −35° C., about −65° C. to about −35° C., about −65° C. to about −40° C., or about −65° C. to about −45° C.

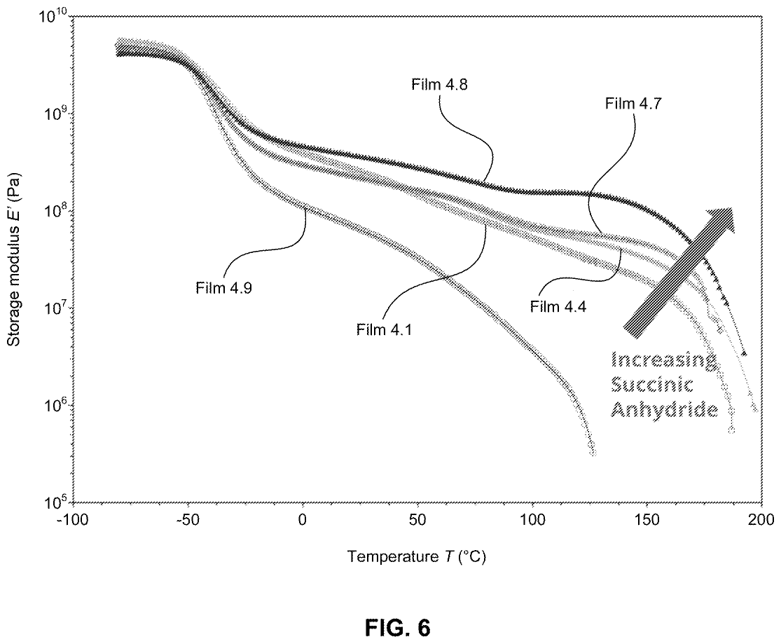

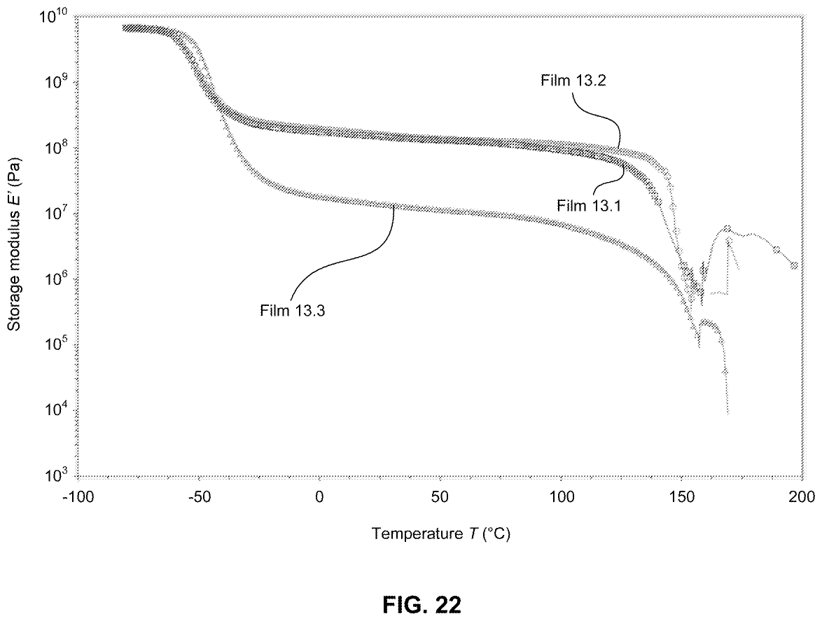

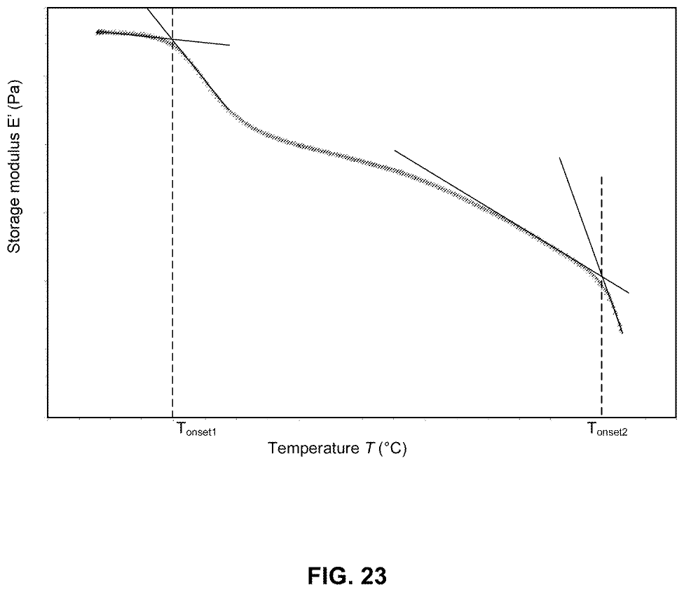

FIGS. 6 and 22 show DMA thermograms for exemplary polyurethanes (Films 4.9 and 13.3). The first DMA modulus transition onset temperature (Tₒₙₛₑₜ₁) for each exemplary polyurethane is the temperature at which the slope of the storage modulus (E′) curve begins to decrease significantly for a first time. The methodology of measuring this value is exemplified in FIG. 23. DMA equipment, such a DMA-850 from TA Instruments, can be programed to calculate this temperature automatically.

Table 1 below shows first DMA modulus transition onset temperatures for some additional exemplary polyurethanes. The “L3360” polyurethane is L3360 available from C.L. Hauthaway & Sons Corporation. The “UD-108,” “UD-250,” and “UD-303” polyurethanes are BONDTHANE™ UD-108, UD-250, and UD-303 polyurethanes available from Bond Polymers International. The “Impranil DLS” polyurethane is IMPRANIL® DLS, an aliphatic polyester polyurethane with 50% solids content in water from Covestro. The “Sancure” polyurethane is SANCURE™ 20025F, an aliphatic polyester polyurethane dispersion at 47% solids in water from Lubrizol. The “HD-2001” polyurethane is HD-2001 available from C.L. Hauthaway & Sons Corporation. The “L2996” polyurethane is an aliphatic polycarbonate polyurethane dispersion with 35% solids content in water from Hauthaway & Sons Corporation.

The properties of the exemplary polyurethanes in Table 1 are the measured properties of polyurethane films prepared by mixing 5.5 g of the listed waterborne polyurethane with 10 mL of de-ionized water and stirring at 1000 rpm (rotations per minute) for 30 minutes at 50° C. The solution was then pipetted into a Teflon evaporating dish with a diameter of 10 cm. The dish was dried in an oven at 45° C. overnight. After drying, the dried sample was conditioned at standard reference atmosphere (23° C., 50% humidity) for 24 hours, resulting in a polyurethane film.

For each polyurethane in Table 1, the DMA values shown were measured using a DMA-850 from TA Instruments. A 1 cm×2.5 cm strip was cut from each film using a metal die. The cut film samples were loaded into the film and fiber tension clamp for testing. During testing, a pre-load of 0.01 N was applied to the cut film samples. The instrument was cooled to −80° C., held for 1 minute, then the temperature was ramped at 4° C./minute to 200° C., or until the sample was too weak to be held in tension. During the temperature ramp, the sample was oscillated 0.1% strain at a frequency of 1 Hz.

TABLE 1
Properties of Exemplary Polyurethanes

| | | 1st Modulus | 2nd Modulus |
|---|---|---|---|
| | Tan(δ) peak | Transition | Transition |
| PU | Temp. (° C.) | Onset (° C.) | Onset (° C) |
| L3360 | −33 | −50 | 114.9 |
| UD-108 | −46 | −58 | 127.4 |
| UD-250 | −30 | — | 160.8 |
| UD-303 | −45 | −59 | 158.1 |
| Impranil DLS | −29 | −44 | 146.4 |
| Sancure | −39 | −50 | 49.6 |
| HD-2001 | −20 | −35 | 128 |
| L2996 | −20 | — | 186.2 |

In some embodiments, the DMA tan (8) peak temperature corresponding to the soft phase of a polyurethane can be below 30° C. In some embodiments, the DMA tan (8) peak temperature corresponding to the soft phase of a polyurethane can range from about −60° C. to about 30° C., including subranges. For example, in some embodiments, the DMA tan (8) peak temperature corresponding to the soft phase of a polyurethane can be about −60° C., about −55° C., about −50° C., about −45° C., about −40° C., about −35° C., about −30° C., about −25° C., about −20° C., about −15° C., about −10° C., about −5° C., about −1° C., 0° C., about 1° C., about 5° C., about 10° C., about 15° C., about 20° C., about 25° C., or about 30° C., or within a range having any two of these values as endpoints, inclusive of the endpoints. In some embodiments, the DMA tan (8) peak temperature corresponding to the soft phase of a polyurethane can be about −60° C. to about 30° C., about −60° C. to about 25° C., about −60° C. to about 20° C., about −60° C. to about 15° C., about −60° C. to about 10° C., about −60° C. to about 5° C., about −60° C. to about 1° C. about −60° C. to 0° C., about −60° C. to about −1° C., about −60° C. to about −5° C., about −60° C. to about −10° C., about −60° C. to about −15° C., about −60° C. to about −20° C., about −60° C. to about −25° C., about −60° C. to about −30° C., about −60° C. to about −35° C., or about −60° C. to about −40° C.

Like DMA modulus transition onset temperatures, DMA equipment, such a DMA-850 from TA Instruments, can be programed to calculate this temperature automatically. Table 1 above lists the DMA tan (8) peak temperature automatically calculated for the listed polyurethanes.

In some embodiments, the second DMA modulus transition onset temperature for a polyurethane can be above 30° C. In some embodiments, the second DMA modulus transition onset temperature for a polyurethane can range from about 45° C. to about 165° C. For example, in some embodiments, the second DMA modulus transition onset temperature for a polyurethane can be about 45° C., about 50° C., about 55° C., about 60° C., about 65° C., about 70° C., about 75° C., about 80° C., about 85° C., about 90° C., about 95° C., about 100° C., about 105° C., about 110° C., about 115° C., about 120° C., about 125° C., about 130° C., about 135° C., about 140° C., about 145° C., about 150° C., about 155° C., about 160° C., or about 165° C., or within any range having any two of these values as endpoints, inclusive of the endpoints. In some embodiments, the second DMA modulus transition onset temperature for a polyurethane can be about 45° C. to about 165° C., about 50° C. to about 160° C., about 55° C. to about 155° C., about 60° C. to about 150° C., about 65° C. to about 145° C., about 70° C. to about 140° C., about 75° C. to about 135° C., about 80° C. to about 130° C., about 85° C. to about 125° C., about 90° C. to about 120° C., about 95° C. to about 115° C., or about 100° C. to about 110° C.

The DMA thermograms in FIGS. 6 and 22 show the second DMA modulus transition onset temperatures for exemplary polyurethanes (Films 4.9 and 13.3). The second DMA modulus transition onset temperature (Tₒₙₛₑₜ₂) for each exemplary polyurethane is the temperature at which the slope of the storage modulus (E′) curve begins decrease significantly for a second time. The methodology of measuring this value is exemplified in FIG. 23. DMA equipment, such a DMA-850 from TA Instruments, can be programed to calculate this temperature automatically. Table 1 above shows second DMA modulus transition onset temperatures for some additional exemplary polyurethanes.

In some embodiments, the polyurethane can exhibit crystallinity in the soft phase. This is common in polyether soft segments containing polytetramethylene glycol and some polyester polyols. In such embodiments, the polyurethane can exhibit at least three transitions: the Tg of the soft phase, the melting point of the soft phase, and the modulus transition of the hard phase. Such melting in the soft phase typically occurs between 0° C. and about 60° C., when present. In embodiments exhibiting crystallinity in the soft phase, the protein polyurethane alloy will typically still exhibit the melting in the soft phase because the protein is miscible with the hard phase, leaving the mechanical properties of the soft phase substantially unchanged.

In typical embodiments described herein, the protein polyurethane alloy can have a second DMA modulus transition onset temperature higher than the second DMA modulus transition temperature of the polyurethane in absence of succinylated protein (i.e., the polyurethane by itself). It is believed that this increase in the second DMA modulus transition onset temperature in the alloy is due to the miscibility of the succinylated protein and the hard phase of the polyurethane. This selective miscibility of the succinylated protein is indicated by an increase in the second DMA modulus transition onset temperature without a similar increase in DMA transition temperature of the soft phase (quantified by a first DMA modulus transition onset temperature or the DMA tan (8) peak temperature corresponding to the soft phase). This selective miscibility can be utilized to control properties of the protein polyurethane alloy, for example mechanical and thermal properties.

In some embodiments, the protein polyurethane alloys and/or the layered materials described herein can have a look and feel, as well as mechanical properties, similar to natural leather. For example, the protein polyurethane alloy layer or the layered material including the protein polyurethane alloy layer can have, among other things, haptic properties, aesthetic properties, mechanical/performance properties, manufacturability properties, and/or thermal properties similar to natural leather. Mechanical/performance properties that can be similar to natural leather include, but are not limited to, tensile strength, tear strength, elongation at break, resistance to abrasion, internal cohesion, water resistance, breathability (quantified in some embodiments by a moisture vapor transmission rate measurement), and the ability to be dyed with reactive dyes and to retain color when rubbed (color fastness). Haptic properties that can be similar to natural leather include, but are not limited to, softness, rigidity, coefficient of friction, and compression modulus. Aesthetic properties that can be similar to natural leather include, but are not limited to, dyeability, embossability, aging, color, color depth, and color patterns. Manufacturing properties that can be similar to natural leather include, but are not limited to, the ability to be stitched, cut, skived, and split. Thermal properties that can be similar to natural leather include, but are not limited to, heat resistance and resistance to stiffening or softening over a significantly wide temperature range, for example 25° C. to 100° C.

Desirable properties for the protein polyurethane alloy described herein, include but are not limited to, optical properties, haptic properties, aesthetic properties, thermal properties, mechanical properties, and/or breathability properties. Exemplary thermal properties include heat resistance and resistance to melting, and can be quantified by, for example, measuring the second modulus transition onset temperature (Tₒₙₛₑₜ₂) of a material. Exemplary mechanical properties include abrasion resistance, maximum tensile stress (also referred to as “tensile strength”), and Young's modulus. Unless otherwise specified, maximum tensile stress values and Young's modulus values disclosed herein are measured according to ISO3376 (“Leather—Physical and mechanical tests”), with the exception that the dogbone test samples had an overall width of 10 mm, a gauge length of 15 mm, and a gauge width of 5 mm. Exemplary breathability properties include moisture vapor transmission rate (MVTR) measured in g/m²/24 hr (grams per meters squared per 24 hours). Unless otherwise specified, moisture vapor transmission rates disclosed herein are measured according to the methods provided by ASTM E96—Method B.

In some embodiments, the protein polyurethane alloy can be transparent. In some embodiments, a transparent protein polyurethane alloy can indicate that the protein is miscible with the hard phase of the polyurethane in the alloy. As used herein, a “transparent” material means material having an opacity of about 50% or less. Opacity is measured by placing a sample of material over a white background to measure the Y tristimulus value (“Over white Y”) in reflectance with a spectrometer using the D65 10 degree illuminant. The same sample is then placed over a black background and the measurement is repeated, yielding “Over black Y”. Percent opacity is calculated as “Over black Y” divided by “Over white Y” times 100. 100% opacity is defined as lowest transparency and 0% opacity is defined as the highest transparency.

In some embodiments, the protein polyurethane alloy can be transparent and can have an opacity ranging from 0% to about 50%, including subranges. For example, the transparent protein polyurethane alloy, can have an opacity ranging from 0% to about 40%, 0% to about 30%, 0% to about 20%, 0% to about 10%, or 0% to about 5%. The transparency of the protein polyurethane alloy is evaluated before dying or otherwise coloring the protein polyurethane alloy.

A transparent protein polyurethane alloy can be created by selecting and blending the appropriate combination of one or more succinylated proteins and one or more polyurethanes. While not all combinations of succinylated protein and polyurethane will result in a transparent protein polyurethane alloy, it is within the skill of the ordinarily skilled artisan to identify whether a given blend results in a transparent protein polyurethane alloy in view of this disclosure. In embodiments directed to a layered material including a transparent protein polyurethane alloy layer described herein, the transparent protein polyurethane alloy layer can provide unique characteristics for the layered material. For example, compared to a non-transparent layer, the transparent protein polyurethane alloy layer can provide unique depth of color when dyed. Likewise, the transparent protein polyurethane alloy layer can provide its mechanical properties to the layered material without significantly influencing the aesthetic properties of the material.

In some embodiments, the protein polyurethane alloy can include one or more coloring agents. In some embodiments, the coloring agent can be a colored dye, for example a fiber reactive dye, a direct dye, or a natural dye. Exemplary dyes, include but are not limited to, Azo structure acid dyes, metal complex structure acid dyes, anthraquinone structure acid dyes, and azo/diazo direct dyes. In some embodiments, the coloring agent can be pigment, for example a lake pigment.

Suitable polyurethanes for blending with one or more succinylated proteins according to embodiments described herein include, but are not limited to, aliphatic polyurethanes, aromatic polyurethanes, bio-based polyurethanes, or acrylic acid modified polyurethanes. Suitable polyurethanes are commercially available from manufacturers including Lubrizol, Hauthaway, Stahl, and the like. In some embodiments, a polyurethane for a protein polyurethane alloy can be bio-polyurethane. In some embodiments, the polyurethane is a water-dispersible polyurethane. In some embodiments, the polyurethane can be a polyester polyurethane. In some embodiments, the polyurethane can be a polyether polyurethane. In some embodiments, the polyurethane can be a polycarbonate-based polyurethane. In some embodiments, the polyurethane can be an aliphatic polyester polyurethane. In some embodiments, the polyurethane can be an aliphatic polyether polyurethane. In some embodiments, the polyurethane can be an aliphatic polycarbonate polyurethane. In some embodiments, the polyurethane can be an aromatic polyester polyurethane. In some embodiments, the polyurethane can be an aromatic polyether polyurethane. In some embodiments, the polyurethane can be an aromatic polycarbonate polyurethane.

In some embodiments, the polyurethane can have a soft segment selected from the group consisting of: polyether polyols, polyester polyols, polycarbonate polyols, and mixtures thereof. In some embodiments, the polyurethane can have a hard segment comprising diisocyanates and optionally short chain diols. Suitable diisocyanates can be selected from the group consisting of: aliphatic diicocyanates such as hexamethylene diisocyanate, isophorone diisocyanate; aromatic diisocyanates such as 4,4′ diphenyl methylene diisocyanate, toluene diisocyanate, phenyl diisocyanate, and mixtures thereof. Suitable short chain diols include ethylene glycol, propane diol, butane diol, 2,2 methyl 1,3 propane diol, pentane diol, hexane diol and mixtures thereof. In some embodiments crosslinkers such as multifunctional alcohols, for example, trimethylol propane triol, or diamines such as ethylene diamine or 4,4′ diamino, diphenyl diamine.

Exemplary commercial polyurethanes, include but are not limited to L3360 and Hauthane HD-2001 available from C.L. Hauthaway & Sons Corporation, SANCURE™ polyurethanes available from Lubrizol Corporation, BONDTHANE™ polyurethanes, for example UD-108, UD-250, and UD-303 available from Bond Polymers International, EPOTAL R ECO 3702 and EPOTALR P100 ECO from BASF, and Permutex Evo EX-RC-2214 (RC-2214) from Stahl. L3360 is a aliphatic polyester polyurethane polymer aqueous dispersion having a 35% solids content, a viscosity of 50 to 500 cps (centipoise), and a density of about 8.5 lb/gal (pounds per gallon). HD-2001 is an aliphatic polyester polyurethane polymer aqueous dispersion having a 40% solids content, a viscosity of 50 to 500 cps, and a density of about 8.9 lb/gal. BONDTHANE™ UD-108 is an aliphatic polyether polyurethane polymer aqueous dispersion having a 33% solids content, a viscosity of 300 cps, and a density of 8.7 lb/gal. BONDTHANE™ UD-250 is an aliphatic polyester polyurethane polymer aqueous dispersion having a 35% solids content, a viscosity of 200 cps, and a density of 8.8 lb/gal. BONDTHANE™ UD-303 is an aliphatic polyether polyurethane polymer aqueous dispersion having a 35% solids content, a viscosity of less than 500 cps, and a density of 8.7 lb/gal. EPTOAL® P100 ECO is a polyester polyurethane elastomer aqueous dispersion having approximately 40% solids and a viscosity of about 40 mPas. RC-2214 is an aliphatic polyether polyurethane polymer aqueous dispersion having a 58-60% solids content, a viscosity of 4,000 to 15,000 cps, and a density of about 8.9 lb/gal.

Exemplary bio-based polyurethanes include, but are not limited to, L3360 available from C.L. Hauthaway & Sons Corporation, IMPRANIL R Eco DLS, IMPRANIL R Eco DL 519, IMPRANILR Eco DLP-R, and IMPRAPERMR DL 5249 available from Covestro. IMPRANILR Eco DLS is an anionic, aliphatic polyester polyurethane polymer aqueous dispersion having approximately 50% solids content, a viscosity of less than 1,200 MPa's, and a density of about 1.1 g/cc. IMPRANIL R Eco DL 519 is an anionic, aliphatic polyester polyurethane polymer aqueous dispersion. IMPRANILR Eco DLP-R is an anionic, aliphatic polyester polyurethane polymer aqueous dispersion. IMPRAPERMR DL 5249 is an anionic aliphatic polyester-polyurethane polymer aqueous dispersion.

In some embodiments, the polyurethane can include reactive groups that can be cross-linked with a succinylated protein. Exemplary reactive groups include, but are not limited to, a sulfonate, an aldehyde, a carboxylic acid or ester, a blocked isocyanate, or the like, and combinations thereof. In such embodiments, the polyurethane can be crosslinked to the succinylated protein in the protein polyurethane alloy through the reaction of a reactive group on the protein with the reactive group present in the polyurethane.

Suitable succinylated proteins for blending with one or more polyurethanes according to embodiments described herein include, but are not limited to, collagen, gelatin, bovine serum albumin (BSA), soy proteins, pea protein, egg white albumin, casein, peanut protein, edestin protein, whey protein, karanja protein, cellulase, and hemp. Suitable collagens include, but are not limited to, recombinant collagen (r-Collagen), a recombinant collagen fragment, and extracted collagens. Suitable soy proteins include, but are not limited to, soy protein isolate (SPI), soymeal protein, and soy protein derivatives. In some embodiments, the soy protein isolate can be partially hydrolyzed soy protein isolate. Suitable pea proteins include, but are not limited to, pea protein isolate, and pea protein derivatives. In some embodiments, the pea protein isolate can be partially hydrolyzed pea protein isolate.

Table 2 below lists some exemplary proteins that can be succinylated as described herein, and properties of those proteins before succinylation. The gelatin is gelatin from porcine skin, Type A (Sigma Aldrich G2500). The collagen is extracted bovine collagen purchased from Wuxi BIOT Biology-technology Company. The bovine serum albumin Sigma Aldrich 5470 bovine serum albumin. The r-Collagen is recombinant collagen from Modern Meadow. The soy protein isolate is soy protein isolate purchased from MP Medicals (IC90545625). The pea protein is pea protein powder purchased from Bobs Red Mills (MTX5232). The egg white albumin protein is albumin from chicken egg white (Sigma Aldrich A5253). The casein protein is casein from bovine milk (Sigma Aldrich C7078). The peanut protein is peanut protein powder purchased from Tru-Nut. The whey protein is whey from bovine milk (Sigma Aldrich W1500). Other suitable soy protein isolates include, but are not limited to, soy protein isolate purchased from AMD (Clarisoy 100, 110, 150, 170, 180), or DuPont (SUPRO® XT 55, SUPRO® XT 221D, and SOBIND® Balance). Other suitable pea protein powders include, but are not limited to, pea protein powder purchased from Puris (870 and 870H).

Karanja protein is a protein found in Karanja seeds harvested from Pongamia pinnata trees (also known as Pongamia glabra trees). See Rahman, M., and Netravali, “Green Resin from Forestry Waste Residue ‘Karanja (Pongamia pinnata) Seed Cake’ for Biobased Composite Structures,” ACS Sustainable Chem. Eng., 2:2318-2328 (2014); see also Mandal et al., “Nutritional Evaluation of Proteins from three Non-traditional Seeds with or without Amino Acids Supplementation in Albino Rats,” Proc. Indian natn. Sci. Acad., B50, No. 1, 48-56 (1984). The protein can be extracted from Karanja seeds using a solvent extraction process. Id. In some embodiments, the karanja protein can be karanja protein isolate. In such embodiments, karanja protein isolate can be obtained by alkaline extraction and acid precipitation of defatted karanja seed cake. See Rahman, M., and Netravali, “Green Resin from Forestry Waste Residue ‘Karanja (Pongamia pinnata) Seed Cake’ for Biobased Composite Structures,” ACS Sustainable Chem. Eng., 2:2318-2328 (2014).

Suitable cellulase proteins are listed below in Table 2. The “Cellulase-RG” protein is Native Trichoderma sp. Cellulase available from CREATIVE ENZYMES®. The “Cellulase-IG” protein is laboratory grade cellulase available from Carolina Biological Supply Company.

The 50 KDa recombinant collagen fragment (50 KDa r-Collagen fragment) in Table 2 is a collagen fragment comprising the amino acid sequence listed as SEQ ID NO: 2.

The “dissolution method” listed in Table 2 is an exemplary aqueous solvent in which the succinylated protein can be dissolved in a solution that is miscible with the hard phase of the polyurethane as described herein. Succinylated proteins that can be at least partly dissolved in an aqueous solution are suitable for forming protein polyurethane alloys with polyurethane dispersions.

TABLE 2
Example Proteins

| | | | | | | Amino Acid | Protein |
|---|---|---|---|---|---|---|---|
| Protein | Protein | Dissolution | Molecular | | Isoelectric | Comp.: Lysine | Thermostability |
| Name | Source | Method | Weight | | Point | (g/100 g) | up to 200° C. |
| Gelatin | Porcine | Water | ~100 | KDa | ~4.8 | 2.6 | Yes |
| Collagen | Bovine | Water | ~120 | KDa | — | 2.2 | — |
| BSA | Bovine | Water | ~66 | KDa | ~4.7 | 11.98 | — |
| (Bovine | | | | | | | |
| Serum | | | | | | | |
| Albumin) | | | | | | | |
| r-Collagen | Yeast | Water | ~100 | KDa | — | 3.6 | — |
| Soy protein | Soy | Water + | ~30 to 60 | KDa | ~4.0 to 5.0 | 5.6 | Yes |
| isolate | | NaOH | | | | | |
| Pea Protein | Pea | Water + | ~60 to 80 | KDa | ~4.5 | 7.6 | Yes |
| | | NaOH | | | | | |
| Egg White | Chicken Egg | Water | ~40 | KDa | ~4.8 | 5.7 | Yes |
| Albumin | | | | | | | |
| Casein | Bovine Milk | Water + | ~24 | KDa | ~4.6 | 7.4 | — |
| | | NaOH | | | | | |
| Peanut | Peanut | Water + | ~60 | KDa | ~4.5 | 2.5 | Yes |
| Protein | | NaOH | | | | | |
| Whey | Bovine Milk | Water | ~18 | KDa | ~4.5 to 5.2 | 9.7 | No |
| Karanja | Karanja Seed | Water | — | | — | 14.6 | — |
| protein | | | | | | | |
| isolate | | | | | | | |
| 50 KDa r- | Yeast | Water | ~50 | KDa | ~9.3 | 3.9 | — |
| Collagen | | | | | | | |
| fragment | | | | | | | |
| Cellulase- | Trichoderma | Water | ~20 to 90 | KDa | ~4.6 to 6.9 | — | — |
| RG | reesei | | | | | | |
| Cellulase- | Trichoderma | Water | — | | — | — | — |
| IG | reesei | | | | | | |

In some embodiments, before succinylation, the protein can have one or more of the following properties: (i) a molecular weight within a range described herein (ii) an isoelectric point within a range described below, (iii) an amino acid composition measured in grams of lysine per 100 grams of protein in a range described below, and (iv) protein thermo-stability up to 200° C.

### Protein Molecular Weight

In some embodiments, before succinylation, the protein can have a molecular weight ranging from about 1 KDa (kilodaltons) to about 700 KDa, including subranges. For example, the protein can have a molecular weight ranging from about 1 KDa to about 700 KDa, about 10 KDa to about 700 KDa, about 20 KDa to about 700 KDa, about 50 KDa to about 700 KDa, about 100 KDa to about 700 KDa, about 200 KDa to about 700 KDa, about 300 KDa to about 700 KDa, about 400 KDa to about 700 KDa, about 500 KDa to about 700 KDa, about 600 KDa to about 700 KDa, about 1 KDa to about 600 KDa, about 1 KDa to about 500 KDa, about 1 KDa to about 400 KDa, about 1 KDa to about 300 KDa, about 1 KDA to about 200 KDa, about 1 KDa to about 100 KDa, about 1 KDa to about 50 KDa, about 1 KDa to about 20 KDa, or about 1 KDa to about 10 KDa, or within a range having any two of these values as endpoints, inclusive of the endpoints.

### Protein Isoelectric Point

In some embodiments, before succinylation, the protein can have an isoelectric point ranging from about 4 to about 10, including subranges. For example, the protein can have an isoelectric point ranging from about 4 to about 10, about 4.5 to about 9.5, about 5 to about 9, about 5.5 to about 8.5, about 6 to about 8, about 6.5 to about 7.5, or about 6.5 to about 7, or within a range having any two of these values as endpoints, inclusive of the endpoints. In some embodiments, the protein can have an isoelectric point ranging from about 4 to about 5.

### Protein Amino Acid Composition

In some embodiments, before succinylation, the protein can have an amino acid composition measured in grams of lysine per 100 grams of protein (as referred to as a “lysine weight percent”) ranging from about 0.5 wt % to about 100 wt %, including subranges. For example, the protein can have a lysine weight percent ranging from about 0.5 wt % to about 100 wt %, about 1 wt % to about 100 wt %, about 5 wt % to about 100 wt %, about 10 wt % to about 100 wt %, about 20 wt % to about 100 wt %, about 30 wt % to about 100 wt %, about 40 wt % to about 100 wt %, about 50 wt % to about 100 wt %, about 60 wt % to about 100 wt %, about 70 wt % to about 100 wt %, about 80 wt % to about 100 wt %, or about 90 wt % to about 100 wt %, or within a range having any two of these values as endpoints, inclusive of the endpoints. In some embodiments, the protein can be a polylysine.

In some embodiments, before succinylation, the protein can have a lysine weight percent ranging from about 0.5 wt % to about 20 wt %, including subranges. For example, the protein can have a lysine weight percent ranging from about 0.5 wt % to about 20 wt %, about 1 wt % to about 19 wt %, about 2 wt % to about 18 wt %, about 3 wt % to about 17 wt %, about 4 wt % to about 16 wt %, about 5 wt % to about 15 wt %, about 6 wt % to about 14 wt %, about 7 wt % to about 13 wt %, about 8 wt % to about 12 wt %, about 9 wt % to about 11 wt %, or about 9 wt % to about 10 wt %, or within a range having any two of these values as endpoints, inclusive of the endpoints. In some embodiments, the protein can have a lysine weight percent ranging from about 1 wt % to about 20 wt %. In some embodiments, the protein can have a lysine weight percent ranging from about 5 wt % to about 20 wt %. In some embodiments, the protein can have a lysine weight percent ranging from about 1 wt % to about 12 wt %. In some embodiments, the protein can have a lysine weight percent ranging from about 5 wt % to about 12 wt %. In some embodiments, the protein can have a lysine weight percent ranging from about 1 wt % to about 15 wt %. In some embodiments, the protein can have a lysine weight percent ranging from about 5 wt % to about 15 wt %.

In some embodiments, the succinylated protein can be thermo-stable. In some embodiments, the succinylated protein can be non-thermo-stable. As described herein, protein thermo-stability is determined by a differential scanning calorimetry (DSC), where a pre-dried protein powder (with moisture less than 3%) is scanned from 0° C. to 200° C. In the protein's DSC curves, an endothermic peak larger than 10 mW/mg is determined to be a “denaturation peak”, and the temperature corresponding to the endothermic “denaturation peak” is defined as the “denaturation temperature” of the protein. A protein that is “thermo-stable” means that the protein has denaturation temperature of 200° C. or more. For purposes of the present disclosure, a protein with a denaturation temperature below 200° C. is considered “non-thermo-stable.”

### Protein Dissolution

In some embodiments, before blending with one or more polyurethanes, one or more succinylated proteins can be dissolved in an aqueous solution to form an aqueous protein mixture. In some embodiments, dissolving the succinylated protein in an aqueous solution before blending the protein with one or more polyurethanes can facilitate miscibility of the protein with the hard phase of the one or more polyurethanes. For example, dissolving the succinylated protein in an aqueous solution before blending the protein with one or more polyurethanes can facilitate miscibility of the protein with the hard phase of the polyurethane(s). Not all proteins are naturally miscible with any phase of a polyurethane.

Suitable aqueous solutions include, but are not limited to, water, an aqueous alkali solution, an aqueous acid solution, an aqueous solution including an organic solvent, a urea solution, and mixtures thereof. In some embodiments, the aqueous alkali solution can be a basic solution such as a sodium hydroxide, ammonia or ammonium hydroxide solution. In some embodiments, examples of an acidic aqueous solution can be an acetic acid or hydrochloric acid (HCl) solutions. Suitable organic solvents include, but are not limited to, ethanol, isopropanol, acetone, ethyl acetate, isopropyl acetate, glycerol, and the like. In some embodiments, the protein concentration in the aqueous protein mixture can range from about 10 g/L to about 300 g/L, including subranges. For example, the protein concentration in the aqueous protein mixture can be about 10 g/L, about 20 g/L, about 30 g/L, about 40 g/L, about 50 g/L, about 60 g/L, about 70 g/L, about 80 g/L, about 90 g/L, about 100 g/L, about 150 g/L, about 200 g/L, about 250 g/L, or about 300 g/L, or within a range having any two of these values as endpoints, inclusive of the endpoints. In some embodiments, the protein concentration in the aqueous protein mixture can range from about 10 g/L to about 300 g/L, about 20 g/L to about 250 g/L, about 30 g/L to about 200 g/L, about 40 g/L to about 150 g/L, about 50 g/L to about 100 g/L, about 60 g/L to about 90 g/L, or about 70 g/L to about 80 g/L.

In some embodiments, the amount of succinylated protein in the protein polyurethane alloy can range from about 10 wt % to about 50 wt % of protein, including subranges. For example, in some embodiments, the amount of succinylated protein in the polyurethane alloy range from about 10 wt % to about 50 wt %, about 15 wt % to about 50 wt %, about 20 wt % to about 50 wt %, about 25 wt % to about 50 wt %, about 30 wt % to about 50 wt %, about 35 wt % to about 50 wt %, about 40 wt % to about 50 wt %, about 45 wt % to about 50 wt %, about 10 wt % to about 45 wt %, about 10 wt % to about 40 wt %, about 10 wt % to about 35 wt %, about 10 wt % to about 30 wt %, about 10 wt % to about 25 wt %, about 10 wt % to about 20, or about 10 wt % to about 15%, or within a range having any two of these values as endpoints, inclusive of the endpoints. In some embodiments, the amount of succinylated protein in the protein polyurethane alloy can range from about 20 wt % to about 35 wt %.

In some embodiments, the amount of polyurethane in the protein polyurethane alloy can range from about 50 wt % to about 90 wt %, including subranges. For example, in some embodiments, the amount of polyurethane in the protein polyurethane alloy can range from about 50 wt % to about 90 wt %, about 55 wt % to about 90 wt %, about 60 wt % to about 90 wt %, about 65 wt % to about 90 wt %, about 70 wt % to about 90 wt %, about 75 wt % to about 90 wt %, about 80 wt % to about 90 wt %, about 85 wt % to about 90 wt %, about 50 wt % to about 85 wt %, about 50 wt % to about 80 wt %, about 50 wt % to about 75 wt %, about 50 wt % to about 70 wt %, about 50 wt % to about 65 wt %, about 50 wt % to about 60 wt %, or about 50 wt % to about 55 wt %, or within a range having any two of these values as endpoints, inclusive of the endpoints. In some embodiments, the amount of polyurethane in the protein polyurethane alloy can range from about 65 wt % to about 80 wt %.

Any of the above-listed ranges for the weight percentages of succinylated protein and polyurethane in the protein polyurethane alloy can be combined. For example, in some embodiments, the weight percentages of succinylated protein and polyurethane in the protein polyurethane alloy can be any of the following. The amount of succinylated protein in the polyurethane alloy can range from about 10 wt % to about 50 wt % and the amount of polyurethane in the protein polyurethane alloy can range from about 50 wt % to about 90 wt %. The amount of succinylated protein in the polyurethane alloy can range from about 15 wt % to about 50 wt % and the amount of polyurethane in the protein polyurethane alloy can range from about 50 wt % to about 85 wt %. The amount of succinylated protein in the polyurethane alloy can range from about 20 wt % to about 50 wt % and the amount of polyurethane in the protein polyurethane alloy can range from about 50 wt % to about 80 wt %. The amount of succinylated protein in the polyurethane alloy can range from about 25 wt % to about 50 wt % and the amount of polyurethane in the protein polyurethane alloy can range from about 50 wt % to about 75 wt %. The amount of succinylated protein in the polyurethane alloy can range from about 30 wt % to about 50 wt % and the amount of polyurethane in the protein polyurethane alloy can range from about 50 wt % to about 70 wt %. The amount of succinylated protein in the polyurethane alloy can range from about 10 wt % to about 40 wt % and the amount of polyurethane in the protein polyurethane alloy can range from about 60 wt % to about 90 wt %. The amount of succinylated protein in the polyurethane alloy can range from about 15 wt % to about 40 wt % and the amount of polyurethane in the protein polyurethane alloy can range from about 60 wt % to about 85 wt %. The amount of succinylated protein in the polyurethane alloy can range from about 20 wt % to about 40 wt % and the amount of polyurethane in the protein polyurethane alloy can range from about 60 wt % to about 80 wt %. The amount of succinylated protein in the polyurethane alloy can range from about 20 wt % to about 35 wt % and the amount of polyurethane in the protein polyurethane alloy can range from about 65 wt % to about 80 wt %. In some embodiments, the above-listed weight percent values and ranges can be based on the total weight of the protein polyurethane alloy or protein polyurethane alloy layer. In some embodiments, the above-listed weight percent values and ranges can be based on the total weight of only succinylated protein and polyurethane in a protein polyurethane alloy or protein polyurethane alloy layer. Unless otherwise specified, a weight percent value or range for the polyurethane and the succinylated protein is based on the total weight of only succinylated protein and polyurethane in a protein polyurethane alloy or protein polyurethane alloy layer.

In some embodiments, the sum of the amount of succinylated protein plus the amount of polyurethane in the protein polyurethane alloy can be about 80 wt % or more. For example, in some embodiments, the sum of the amount of succinylated protein plus the amount of polyurethane in the protein polyurethane alloy can range from about 80 wt % to 100 wt %, about 82 wt % to 100 wt %, about 84 wt % to 100 wt %, about 86 wt % to 100 wt %, about 88 wt % to 100 wt %, about 90 wt % to 100 wt %, about 92 wt % to 100 wt %, about 94 wt % to 100 wt %, about 96 wt % to 100 wt %, or about 98 wt % to 100 wt %.

In some embodiments, the protein polyurethane alloy can include water making up a portion of the total weight percent of the material. In some embodiments, the amount of water in the protein polyurethane alloy can range from about 1 wt % to about 10 wt %, including subranges. For example, in some embodiments, the amount of water in the protein polyurethane alloy can range from about 1 wt % to about 10 wt %, about 2 wt % to about 10 wt %, about 3 wt % to about 10 wt %, about 4 wt % to about 10 wt %, about 5 wt % to about 10 wt %, about 6 wt % to about 10 wt %, about 7 wt % to about 10 wt %, about 8 wt % to about 10 wt %, about 1 wt % to about 9 wt %, about 1 wt % to about 8 wt %, about 1 wt % to about 7 wt %, about 1 wt % to about 6 wt %, about 1 wt % to about 5 wt %, about 1 wt % to about 4 wt %, or about 1 wt % to about 3 wt %, or within a range having any two of these values as endpoints, inclusive of the endpoints.

A protein polyurethane alloy described herein can have one or more of (i) a second Dynamic Mechanical Analysis (DMA) modulus transition onset temperature greater than the second DMA modulus transition onset temperature of the unalloyed polyurethane (ii) a first Dynamic Mechanical Analysis (DMA) modulus transition onset temperature substantially the same as the first DMA modulus transition onset temperature of the unalloyed polyurethane, (iii) a DMA tan (8) peak at a temperature substantially the same as the temperature of the DMA tan (8) peak corresponding to the soft phase of the unalloyed polyurethane, or (iv) a moisture vapor transmission rate (MVTR) greater than the MVTR of the unalloyed polyurethane.

Further, as discussed above, succinylation as described herein can increase the solubility of a protein in water and/or increase the average lysine modification of a protein, compared to a non-succinylated protein. Without wishing to be bound by theory, it is believed these characteristics imparted by succinylation enhance the interaction of the succinylated protein with the hard phase of a polyurethane. By increasing the interaction with the hard phase, it is believed the dissolution of the succinylated protein within the hard phase can be enhanced compared to a non-succinylated protein.

The enhanced interaction with the hard phase in a protein polyurethane can be quantified by one or more of: a higher second Dynamic Mechanical Analysis (DMA) modulus transition onset temperature, (ii) a higher Young's modulus, (iii) a higher tensile strength, or (iv) a higher moisture vapor transmission rate (MVTR) for protein polyurethane alloy made with a succylinated protein compared to a protein polyurethane alloy made with the same protein, but not succinylated. Furthermore, the increased interaction with the polyurethane's hard phase can result in improved aesthetic properties, for example improved color stability, improved ability to accept and display the intended color of a dye, or both.

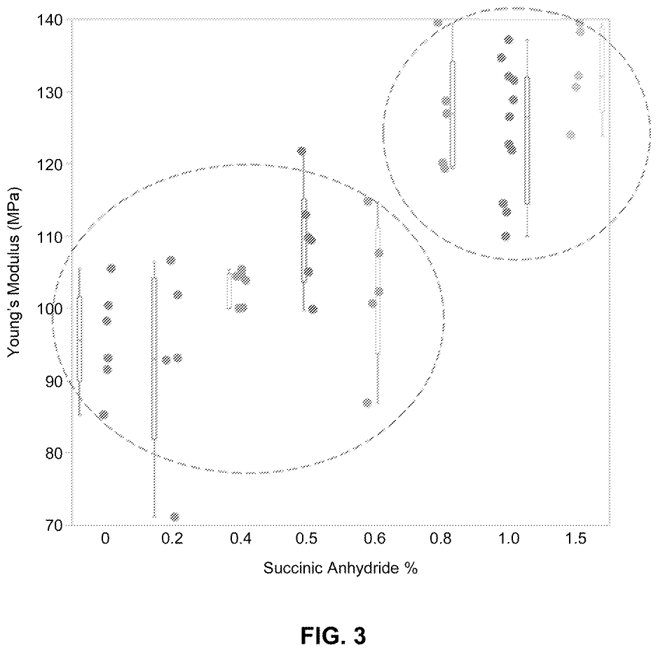

FIGS. 3-22 illustrate the effects of dissolving succinylated soy protein isolates in water and dissolving succinylated soy protein isolates in polyurethanes to form protein polyurethane alloys according to some embodiments. Example Nos. 4-12 detail each soy protein isolate solution and protein polyurethane alloy evaluated.

With respect to second DMA modulus transition onset temperature, FIGS. 6 and 22 show that protein polyurethane alloys made with succinylated soy protein isolate can exhibit higher DMA modulus transition onset temperatures compared to an unalloyed polyurethane, a protein polyurethane alloy made with non-succinylated soy protein isolate, or both.

As shown in FIG. 6, an unalloyed Film 4.9 had a second DMA modulus transition onset temperature of about 111.72° C., while alloyed Films 4.4, 4.7, and 4.8 made with succinylated soy protein isolate had a second DMA modulus transition onset temperature of 168.0° C., 168.1° C., and 169.5° C., respectively. This is an increase of about 58° C. compared to the unalloyed Film 4.9. Compared to an alloyed Film 4.1 made with non-succinylated soy protein isolate, the alloyed Films 4.4, 4.7, and 4.8 showed an increase in second DMA modulus transition onset temperature of about 2° C. Further, Film 4.1 had a storage modulus of about 35 MPa at 125° C., whereas Films 4.4, 4.7, and 4.8 had a storage modulus of about 52 MPa, 58 MPa, and 154 MPa at 125° C. This change in storage modulus at 125° C. indicates the succinylated soy protein can have enhanced interaction with the polyurethane's hard phase in the alloy.

Similarly, as shown in FIG. 22, Films 13.1 and 13.2 made with non-succinylated soy protein isolate and succinylated soy protein isolate, respectively, showed improved thermostability compared to an unalloyed polyurethane Film 13.3. Although Films 13.1 and 13.2 did not show a significant increase in second DMA modulus transition onset temperature compared Film 13.3, the widening of the rubbery plateau in the thermogram indicates the succinylated and non-succinylated soy protein isolate were dissolved in polyurethane's hard phase in the alloy.

With respect to Young's modulus, FIGS. 3, 12, and 19 show that protein polyurethane alloys made with succinylated soy protein isolate can exhibit a Young's modulus substantially the same or higher than a protein polyurethane alloy made with a non-succinylated soy protein isolate. As shown in FIG. 3, protein polyurethane alloys made with succinylated soy protein isolate (data points corresponding to 0.2-1.5% succinic anhydride (SA) (average lysine modification of about 23.3% to about 94.17%, solubility of about 51.5% to about 95.33%, see Table 3)) had a Young's modulus ranging from about 90 MPa to about 135 MPa. Similarly, a protein polyurethane alloy made with non-succinylated soy protein isolate (data points corresponding to 0% SA) had a Young's modulus of about 95 MPa. For polyurethane alloys made with soy protein isolate succinylated at 0.8-1.5% SA (average lysine modification of about 81.8% to about 94.17%, solubility of about 87.10% to about 95.33%, see Table 3), the alloys had a Young's modulus ranging from about 125 MPa to about 135 MPa, which represents an increase in Young's modulus of about 30 MPa to about 40 MPa compared to the protein polyurethane alloy made with non-succinylated soy protein isolate.

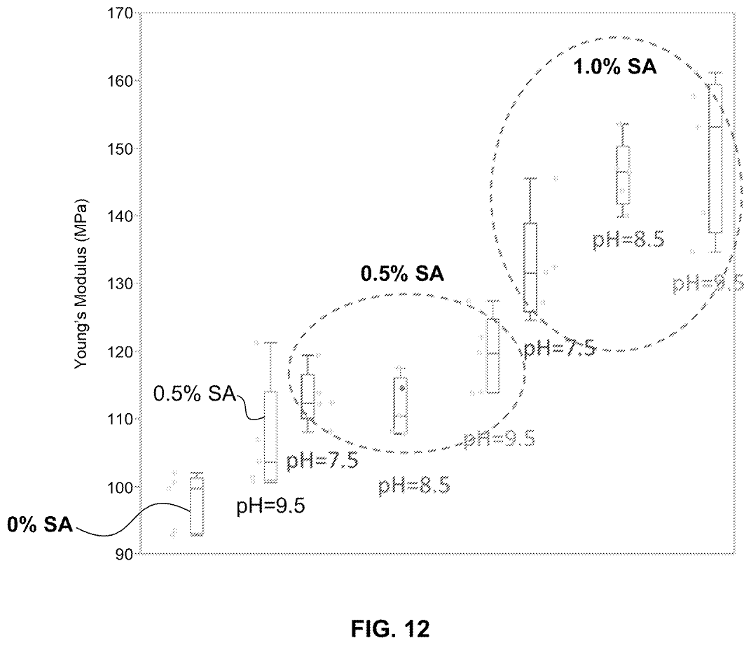

As shown in FIG. 12, protein polyurethane alloys made with succinylated soy protein isolate succinylated at different pHs had a Young's modulus ranging from about 100 MPa to about 155 MPa. Similarly, a protein polyurethane alloy made with non-succinylated soy protein isolate had a Young's Modulus of about 100 MPa. For polyurethane alloys made with soy protein isolate succinylated at 1.0% SA, the alloys had a Young's modulus ranging from about 130 MPa to about 150 MPa, which represents an increase in Young's modulus of about 30 MPa to about 50 MPa compared to the protein polyurethane alloy made with non-succinylated soy protein isolate. FIG. 12 also illustrates that the pH at which a soy protein isolate is succinylated can change the Young's modulus of a protein polyurethane alloy made with succinylated soy protein isolate.

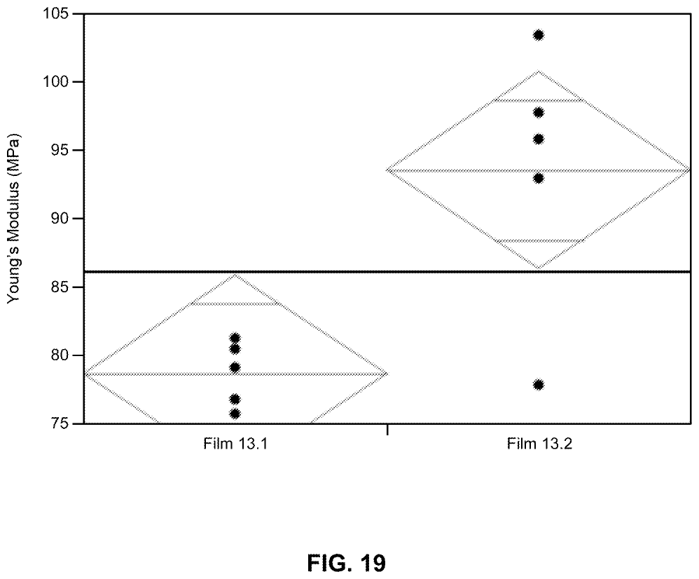

As shown in FIG. 19, a protein polyurethane alloy made with succinylated soy protein isolate (Film 13.2) can exhibit a higher Young's modulus compared to the same protein polyurethane alloy made with non-succinylated soy protein isolate (13.1). Film 13.2 had a Young's modulus of about 93 MPa, whereas Film 13.1 had a Young's modulus of about 78 MPa.

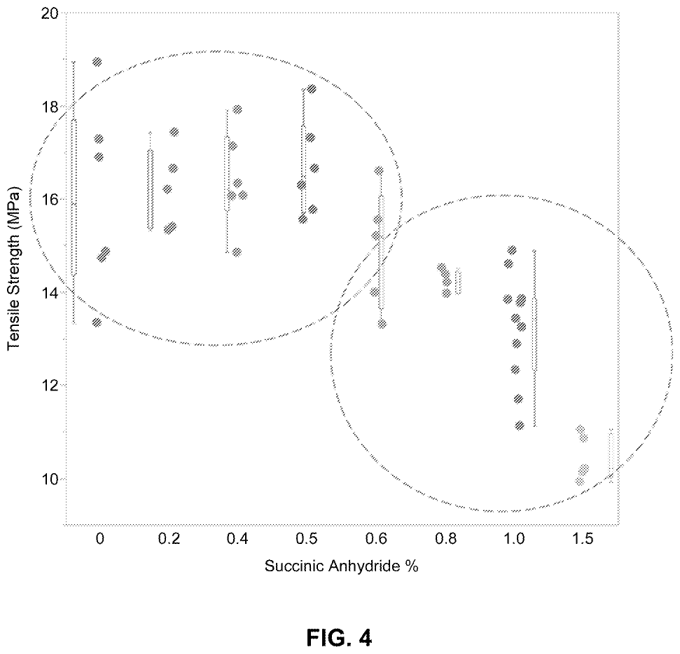

With respect to tensile strength, FIGS. 4, 13, and 20 show that protein polyurethane alloys made with succinylated soy protein isolate can exhibit a tensile strength substantially the same or higher than a protein polyurethane alloy made with a non-succinylated soy protein isolate. As shown in FIG. 4, protein polyurethane alloys made with succinylated soy protein isolate (data points corresponding to 0.2-1.5% succinic anhydride (SA) (average lysine modification of about 23.3% to about 94.17%, solubility of about 51.5% to about 95.33%, see Table 3)) had a tensile strength ranging from about 10 MPa to about 16.5 MPa. Similarly, a protein polyurethane alloy made with non-succinylated soy protein isolate (data points corresponding to 0% SA) had a tensile strength of about 16 MPa.

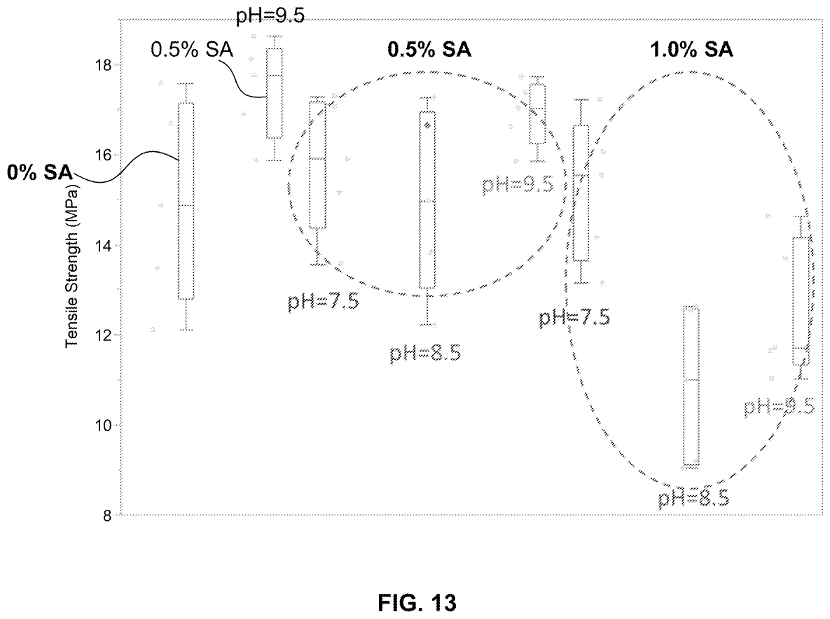

As shown in FIG. 13, protein polyurethane alloys made with succinylated soy protein isolate succinylated at different pHs had a tensile strength ranging from about 11 MPa to about 18 MPa. Similarly, a protein polyurethane alloy made with non-succinylated soy protein isolate had a tensile strength of about 15 MPa. FIG. 13 also illustrates that the pH at which a soy protein isolate is succinylated can change the tensile strength of a protein polyurethane alloy made with succinylated soy protein isolate.

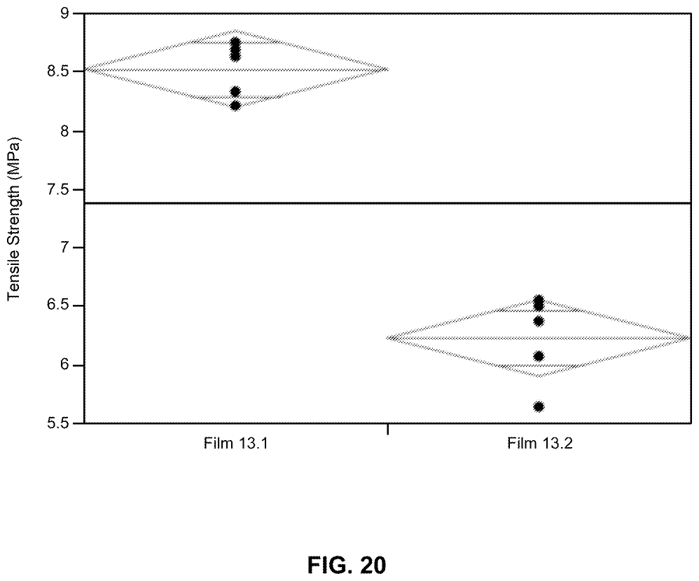

As shown in FIG. 20, a protein polyurethane alloy made with succinylated soy protein isolate (Film 13.2) can exhibit a higher tensile strength compared to the same protein polyurethane alloy made with non-succinylated soy protein isolate (Film 13.1). Film 13.2 had a tensile strength of about 8.5 MPa, whereas Film 13.1 had a tensile strength of about 6.25 MPa.

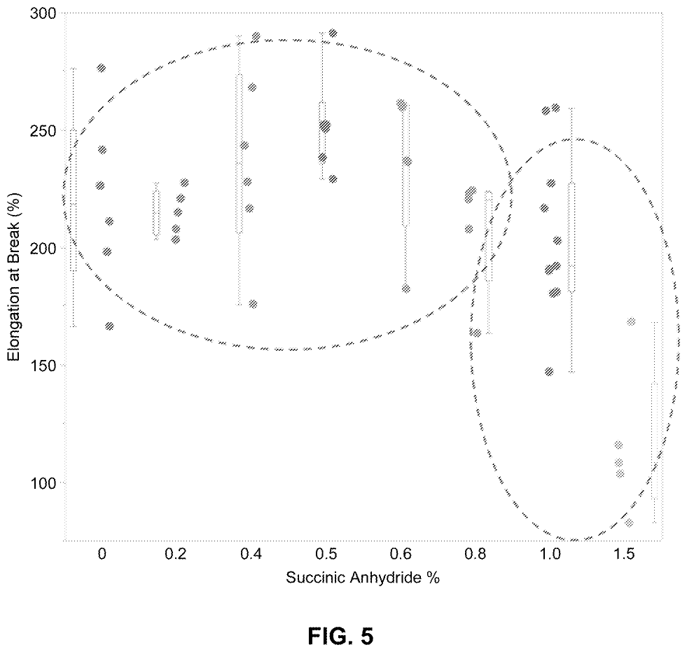

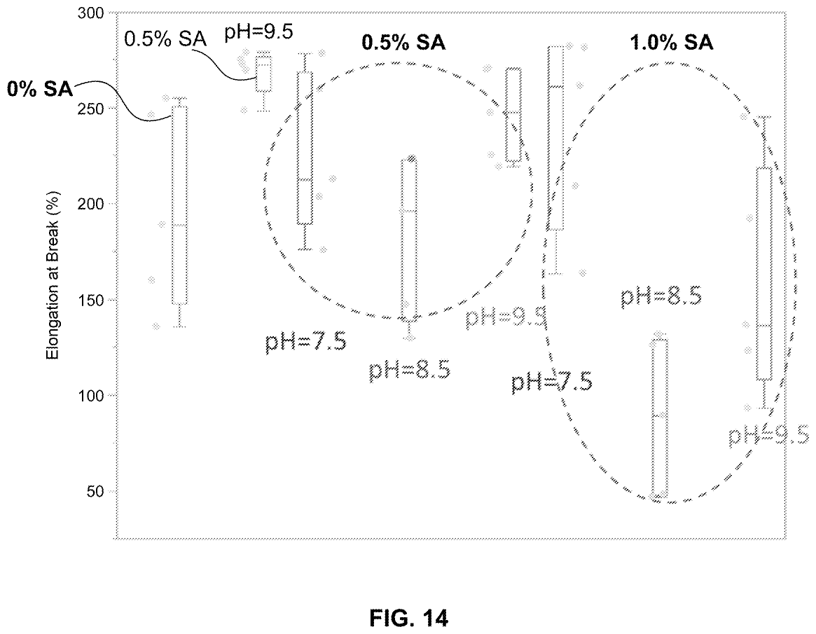

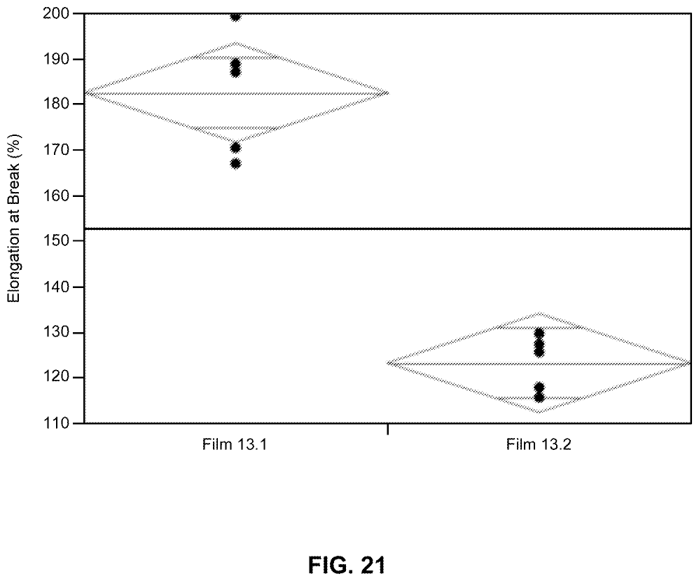

FIGS. 5, 14, and 21 show that protein polyurethane alloys made with succinylated soy protein isolate can exhibit a different elongation at break compared to a protein polyurethane alloy made with a non-succinylated protein. As shown in FIG. 5, protein polyurethane alloys made with succinylated soy protein isolate (data points corresponding to 0.2-1.5% succinic anhydride (SA) (average lysine modification of about 23.3% to about 94.17%, solubility of about 51.5% to about 95.33%, see Table 3)) had an elongation at break ranging from about 115% to about 250% A protein polyurethane alloy made with non-succinylated soy protein isolate (data points corresponding to 0% SA) had an elongation at break of about 220%. As shown in FIG. 14, protein polyurethane alloys made with soy protein isolate succinylated at different pHs had an elongation at break ranging from about 90% to about 270%, while a protein polyurethane alloy made with non-succinylated soy protein isolate (data points corresponding to 0% SA) had an elongation at break of about 190%. FIG. 21 shows a protein polyurethane alloy made with succinylated soy protein isolate (Film 13.2) can exhibit a higher elongation at break compared to the same protein polyurethane alloy made with non-succinylated soy protein isolate (Film 13.1). Film 13.2 had an elongation at break of about 182%, whereas Film 13.1 had an elongation at break of about 125%.

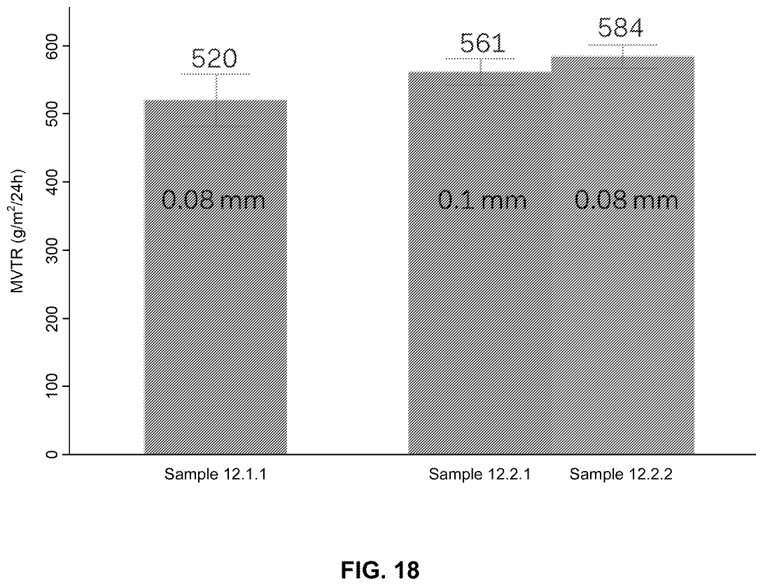

With respect to moisture vapor transmission rate (MVTR), FIG. 18 shows that protein polyurethane alloys made with succinylated soy protein isolate can exhibit higher MVTR compared to an unalloyed polyurethane, a protein polyurethane alloy made with a non-succinylated soy protein isolate, or both. Protein polyurethane alloys made with succinylated soy protein isolate (Samples 12.2.1 and 12.2.2 (average lysine modification of about 68.49%, solubility of about 73.73, see Table 3)) had a MVTR of about 561 g/m²/24 hr and 584 g/m²/24 hr, respectively, which is an increase of about 40 to 60 g/m²/24 hr compared to a protein polyurethane alloy made with non-succinylated soy protein isolate (Sample 12.1.1). For purposes of comparison, an unalloyed polyurethane film made with the same polyurethane as Samples 12.1.1, 12.2.1, 12.2.2 can have a MVTR of about 338 g/m²/24 hr at a thickness of 0.08 mm. As such, protein polyurethane alloys made with succinylated soy protein isolate can exhibited improved MVTR compared to an unalloyed polyurethane film made of the same polyurethane. The MVTR ranging from about 561 g/m²/24 hr to 584 g/m²/24 hr for the succinylated soy protein isolate alloys represents an increase in MVTR of about 200 g/m²/24 hr to 300 g/m²/24 hr compared to the unalloyed polyurethane film made of the same polyurethane.

The unalloyed polyurethane film having a MVTR of about 338 g/m²/24 hr was made by the following process. The film was prepared by mixing 0.4 g of AF-715 (an antifoaming agent available from Quaker Color) into 38 g of waterborne polyurethane dispersion IMPRAPERM® DL 5249 from Covestro. The mixture was mixed using an impeller at a rate of 500 rpm and allowed to stir for 5 minutes at room temperature. After the mixture was properly mixed, 0.6 g BORCHIR Gel L 75 N was added to increase the viscosity of the mixture and the mixture was allowed to mix for 5 minutes. The mixture was then coated using a Mathis LTE-S Labcoater coater onto a release paper and was dried at 75° C. for 10 minutes and at 100° C. for 10 minutes. The coating was then removed from the release paper to create a polyurethane film containing no protein.

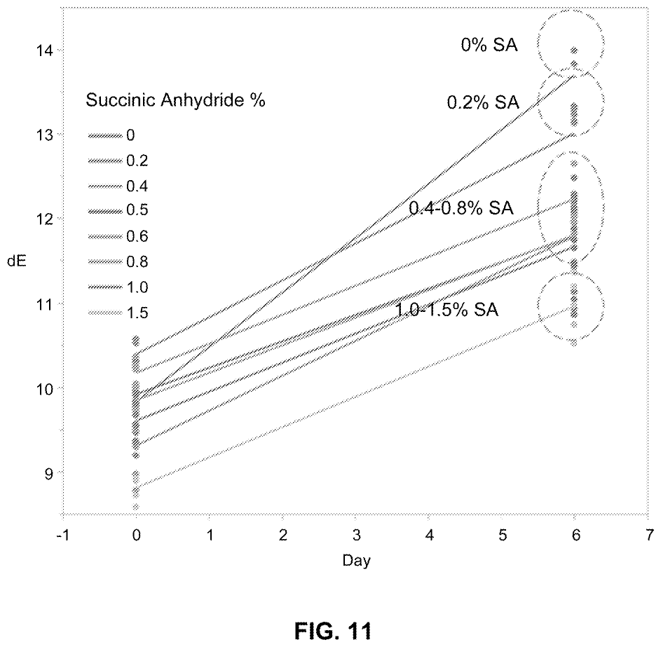

Improved aesthetic properties of protein polyurethane alloys made with succinylated proteins according to embodiments described herein are exemplified in FIGS. 11, 16, 17A, and 17B.

FIG. 11 shows improved color stability for protein polyurethane alloys made with succinylated soy protein isolate (data points corresponding to 0.2-1.5% succinic anhydride (SA) (average lysine modification of about 23.3% to about 94.17%, solubility of about 51.5% to about 95.33%, see Table 3)) compared to a protein polyurethane alloy made with non-succinylated soy protein isolate (data points corresponding to 0% SA). All the protein polyurethane alloys made with succinylated soy protein isolate had a smaller change in color (ΔE, dE*) after hydrolysis for six days compared to the alloy made with non-succinylated soy protein isolate.

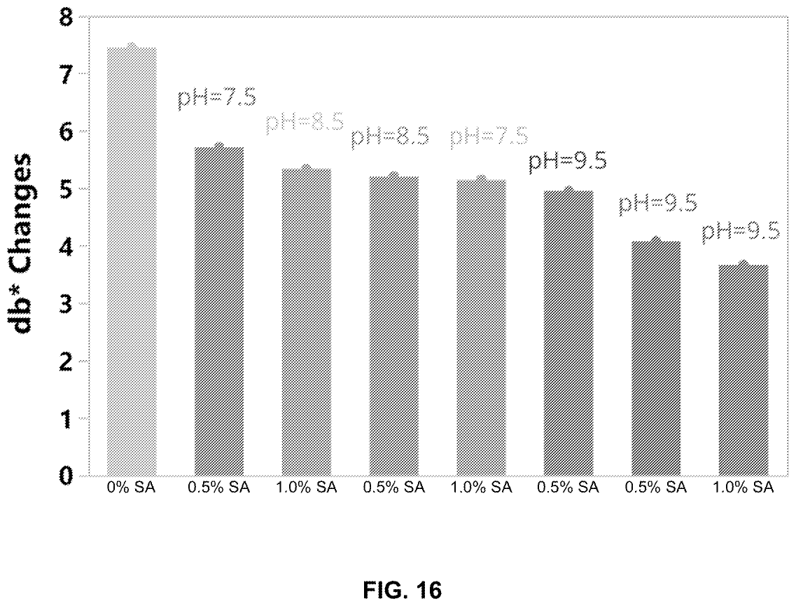

FIG. 16 shows improved color stability for protein polyurethane alloys made with soy protein isolate succinylated at different pHs (data points corresponding to 0.5 and 1.0% succinic anhydride (SA)) compared to a protein polyurethane alloy made with non-succinylated soy protein isolate (data point corresponding to 0% succinic anhydride (SA)). The protein polyurethane alloys made with succinylated soy protein isolate had a smaller change in blueness-yellowness difference (Δb*, db*) after hydrolysis for six days compared to the alloy made with non-succinylated soy protein isolate.

In some embodiments, the protein polyurethane alloys described herein can have a color stability defined by a change in a spectrometer measured b* value in the CIELab color space of 6 or less, wherein the change in the spectrometer measured b* value is a change in b* over six days with the protein polyurethane alloy placed in a hydrolysis chamber at 70° C. and 95% humidity. In some embodiments, the protein polyurethane alloys described herein can have a color stability defined by a change in a spectrometer measured b* value in the CIELab color space of greater than or equal to 3 to less than or equal to 6, wherein the change in the spectrometer measured b* value is a change in b* over six days with the protein polyurethane alloy placed in a hydrolysis chamber at 70° C. and 95% humidity.

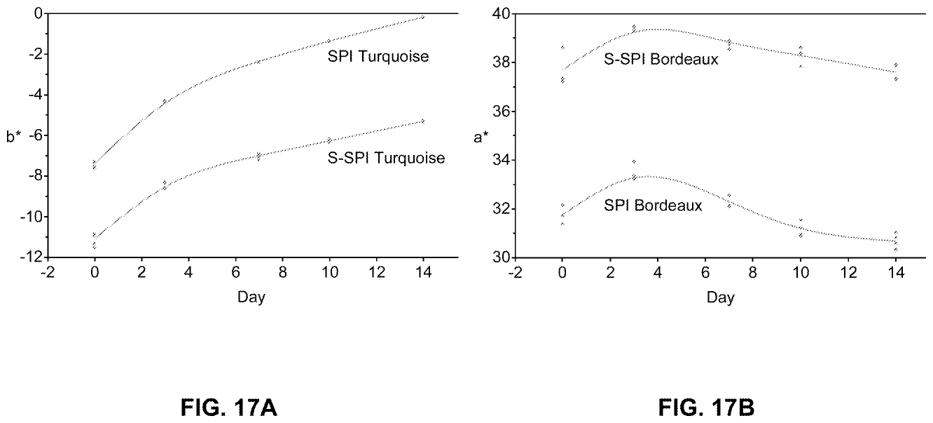

FIGS. 17A and 17B show that a protein polyurethane alloy made with succinylated soy protein isolate can exhibit an improved ability to accept and display the intended color of a dye compared to a protein polyurethane alloy made with non-succinylated soy protein isolate.

FIG. 17A shows the change in measured b* values for a turquoise-dyed protein polyurethane alloy made with succinylated soy protein isolate and a turquoise-dyed protein polyurethane alloy made with non-succinylated soy protein isolate over a 14 day period. FIG. 17B shows the change in measured a* values for a bordeaux-dyed protein polyurethane alloy made with succinylated soy protein isolate and a bordeaux-dyed protein polyurethane alloy made with non-succinylated soy protein isolate over a 14 day period. The turquoise-dyed and bordeaux-dyed alloys were placed in a hydrolysis chamber at 70° C. and 95% humidity between measurements. The alloy made with succinylated soy protein isolate exhibited a lower (more negative) b* value at each measurement, which indicates the alloy was superior at accepting and displaying the turquoise dye color initially and over time. Similarly, the alloy made with succinylated soy protein isolate exhibited a higher a* value at each measurement, which indicates the alloy was superior at accepting and displaying the Bordeaux dye color initially and over time.

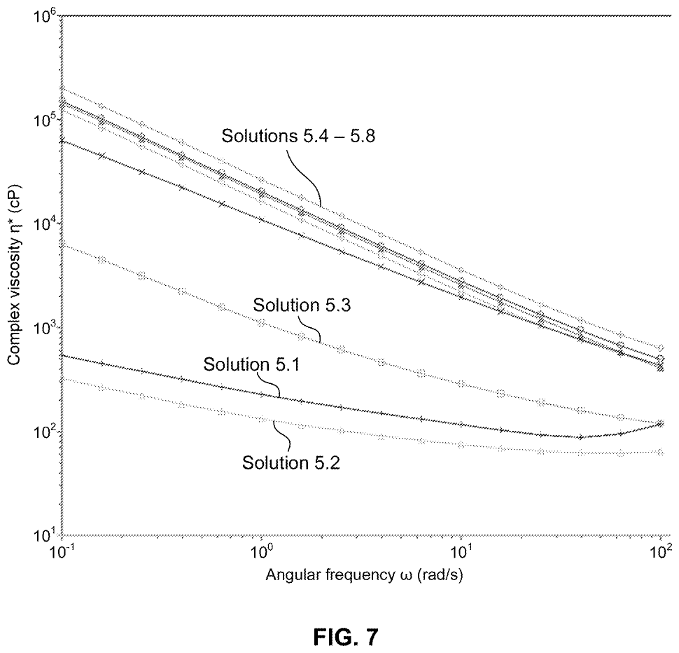

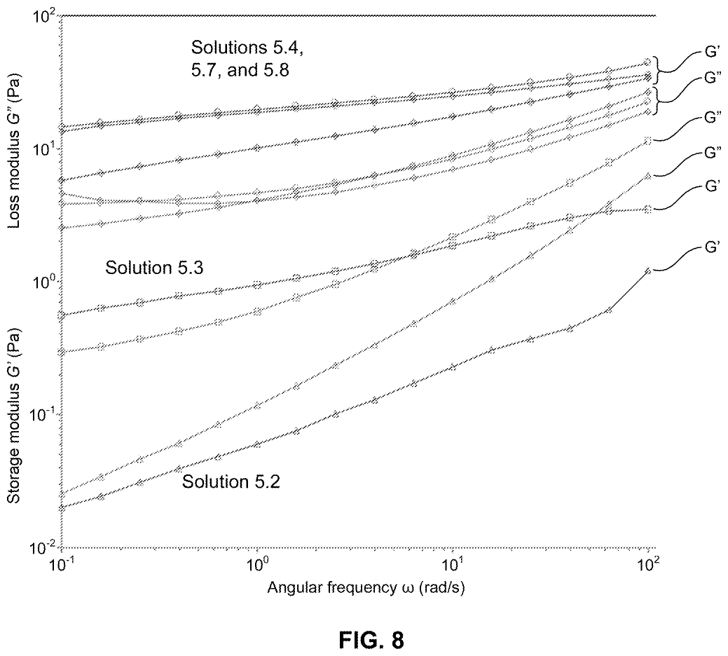

The enhanced solubility of a succinylated protein as described herein is exemplified in FIGS. 7 and 8, which show that complex viscosity, as well as storage and loss moduli, of aqueous solutions made with soy protein isolate increase because of succinylation. As shown in FIG. 7, aqueous protein solutions made with soy protein isolate succinylated at 0.5 to 1.5 w/v % succinic anhydride (SA) (Solutions 5.4-5.8, average lysine modification of about 68.49% to about 94.17%, solubility of about 73.73% to about 95.33%, see Table 3)) exhibited increased complex viscosity compared to aqueous protein solutions made with non-succinylated soy protein isolate (Solution 5.1) and soy protein isolate succinylated at 0.1 and 0.3 w/v % SA (Solutions 5.2 and 5.3, average lysine modification of about 23.3% to about 41.4%, solubility of about 51.5% to about 57.1%, see Table 3)). Relatedly, as shown in FIG. 8, aqueous protein solutions made with soy protein isolate succinylated at 0.5, 1.0. and 1.5 w/v % SA (Solutions 5.4-5.8) exhibited increased storage and loss moduli compared to aqueous protein solutions made with soy protein isolate succinylated at 0.1 and 0.3 w/v % SA.

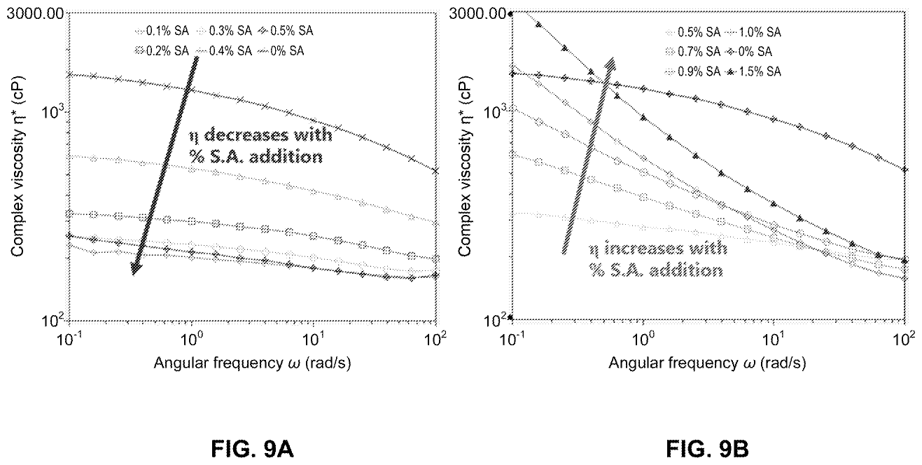

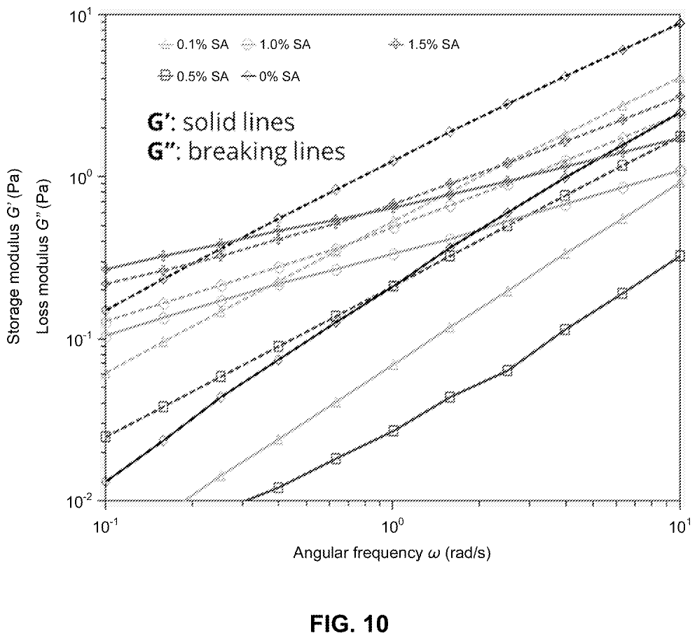

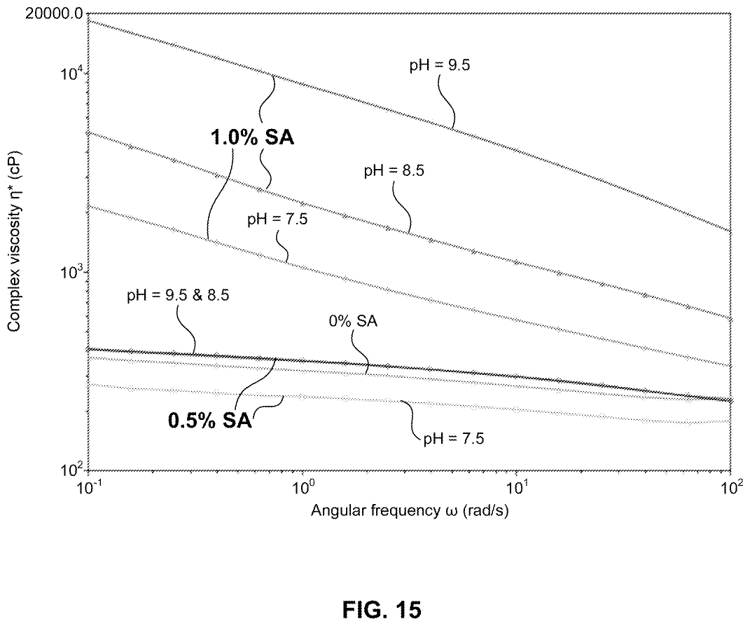

Further, the enhanced interaction between a succinylated protein and the hard phase of a polyurethane is exemplified in FIGS. 9A, 9B, 10, and 15, which show that complex viscosity, as well as storage and loss moduli, of protein polyurethane alloy solutions made with soy protein isolate change based on succinylation conditions. As shown in FIGS. 9A and 9B, protein polyurethane alloy solutions made with soy protein isolate succinylated at 0.1 to 0.5 w/v % succinic anhydride (average lysine modification of about 23.3% to about 68.49%, solubility of about 51.5% to about 73.73%, see Table 3) displayed a Newtonian behavior while the alloy solutions made with soy protein isolate succinylated at 0.5 to 1.5 w/v % succinic anhydride (average lysine modification of about 68.49% to about 94.17%, solubility of about 73.73% to about 95.33%, see Table 3) displayed a shear thinning behavior. Relatedly, as shown in FIG. 10, shows that the storage modulus (G′) and loss modulus (G″) for protein polyurethane alloy solutions made with succinylated soy protein isolate increased as the w/v % of succinic anhydride (SA) used to succinylate the soy protein isolate increased. While the solution made with soy protein isolate succinylated at 1.5 w/v % succinic anhydride had similar viscosity to the solution made with non-succinylated soy protein isolate (see FIGS. 9A and 9B), its storage modulus (G′) was higher than its loss modulus (G″). Without wishing to be bound by theory, these trends in complex viscosity, loss modulus, and storage modulus suggests there may be increased network between the polyurethane and the protein due to succinylation, which may indicate enhanced interaction between the protein and the polyurethane's hard phase within the alloy. FIG. 15 illustrates that the pH at which a soy protein isolate is succinylated can change the complex viscosity of a protein polyurethane solution made with succinylated soy protein isolate.

In some embodiments, the protein polyurethane alloy can comprise a polyurethane having a second DMA modulus transition onset temperature in the absence of protein. That same protein polyurethane alloy can have a second DMA modulus transition onset temperature ranging from about 15° C. to about 100° C. greater than the second DMA modulus transition onset temperature of the polyurethane in the absence of protein. This relative increase in the second DMA modulus transition onset temperature can be referred to as “Delta 2″d Modulus Transition Onset.” In some embodiments, the Delta 2^{nd} Modulus Transition Onset can be about 15° C. or more. In some embodiments, the Delta 2^{nd} Modulus Transition Onset can range from about 15° C. to about 100° C., about 15° C. to about 95° C., about 15° C. to about 90° C., about 15° C. to about 85° C., about 15° C. to about 80° C., about 15° C. to about 75° C., about 15° C. to about 70° C., about 15° C. to about 65° C., about 15° C. to about 60° C., about 15° C. to about 55° C., about 15° C. to about 50° C., about 15° C. to about 45° C., about 15° C. to about 40° C., about 15° C. to about 35° C., about 15° C. to about 30° C., about 15° C. to about 25° C., about 15° C. to about 20° C., about 20° C. to about 100° C., about 25° C. to about 100° C., about 30° C. to about 100° C., about 35° C. to about 100° C., about 40° C. to about 100° C., about 45° C. to about 100° C., about 50° C. to about 100° C., about 55° C. to about 100, about 60° C. to about 100° C., about 65° C. to about 100° C., about 70° C. to about 100° C., about 75° C. to about 100° C., about 80° C. to about 100° C., about 85° C. to about 100° C., about 90° C. to about 100° C., or about 95° C. to about 100° C., or within an range having any two of these values as endpoints, inclusive of the endpoints.

In some embodiments, the protein polyurethane alloy can have a second DMA modulus transition onset temperature ranging from about 130° C. to about 200° C., including subranges. For example, in some embodiments, the protein polyurethane alloy can have a second DMA modulus transition onset temperature ranging from about 130° C. to about 200° C., about 130° C. to about 195° C., about 130° C. to about 190° C., about 130° C. to about 185° C., about 130° C. to about 180° C., about 130° C. to about 175° C., about 130° C. to about 170° C., about 130° C. to about 165° C., about 130° C. to about 160° C., about 130° C. to about 155° C., about 130° C. to about 150° C., about 130° C. to about 145° C., about 130° C. to about 140° C., about 130° C. to about 135° C., about 135° C. to about 200° C., about 140° C. to about 200° C., about 145° C. to about 200° C., about 150° C. to about 200° C., about 155° C. to about 200° C., about 160° C. to about 200° C., about 165° C. to about 200° C., about 170° C. to about 200° C., about 175° C. to about 200° C., or about 180° C. to about 200° C., or within a range having any two of these values as endpoints, inclusive of the endpoints.

In some embodiments, the protein polyurethane alloy can have a first DMA modulus transition temperature below 30° C. In some embodiments, the protein polyurethane alloy can have a first DMA modulus transition onset temperature ranging from about −65° C. to about 30° C., including subranges. For example, in some embodiments, the first DMA modulus transition onset temperature for the protein polyurethane alloy can range from about −65° C. to about 30° C., about −65° C. to about 25° C., about −65° C. to about 20° C., about −65° C. to about 15° C., about −65° C. to about 10° C., about −65° C. to about 5° C., about −65° C. to about 1° C., about −65° C. to 0° C., about-65° C. to about 1° C., about −65° C. to about −5° C., about −65° C. to about −10° C., about −65° C. to about −15° C., about −65° C. to about −20° C., about −65° C. to about −25° C., about −65° C. to about −30° C., about −65° C. to about −35° C., about −65° C. to about −35° C., about −65° C. to about −40° C., or about −65° C. to about −45° C., or within a range having any two of these values as endpoints, inclusive of the end points.

In some embodiments, the protein polyurethane alloy can comprise a polyurethane having a first DMA modulus transition onset temperature in the absence of protein. That same protein polyurethane alloy can have a first DMA modulus transition onset temperature that is +/−X° C. the first DMA modulus transition onset temperature of the polyurethane in the absence of protein. This relative increase or decrease in the first DMA modulus transition onset temperature can be referred to as “Delta 1^{st} Modulus Transition Onset.” In some embodiments, X can be 1, 2, 3, 4, 5, 6, 7, 8, 9, or 10.

In some embodiments, the protein polyurethane alloy can have a DMA tan (8) peak temperature below 30° C. In some embodiments, the protein polyurethane alloy can have a DMA tan (8) peak temperature ranging from about −60° C. to about 30° C., including subranges. For example, in some embodiments, the DMA tan (8) peak temperature for the protein polyurethane alloy can range from about −60° C. to about 30° C., about −60° C. to about 25° C., about −60° C. to about 20° C., about −60° C. to about 15° C., about −60° C. to about 10° C., about −60° C. to about 5° C., about −60° C. to about 1° C., about −60° C. to 0° C., about −60° C. to about 1° C., about −60° C. to about −5° C., about −60° C. to about −10° C., about −60° C. to about −15° C., about −60° C. to about −20° C., about −60° C. to about −25° C., about −60° C. to about −30° C., about −60° C. to about −35° C., or about −60° C. to about −40° C., or within a range having any two of these values as endpoints.

In some embodiments, the protein polyurethane alloy can include a polyurethane having a DMA tan (8) peak temperature corresponding to the soft phase of the polyurethane in the absence of protein. That same the protein polyurethane alloy can have a DMA tan (8) peak temperature that is +/−Y° C. the DMA tan (8) peak temperature corresponding to the soft phase of the polyurethane in the absence of protein. This relative increase or decrease in the DMA tan (8) peak temperature can be referred to as “Delta Tan (8) Peak Temperature.” In some embodiments, Y can be 1, 2, 3, 4, 5, 6, 7, 8, 9, or 10.

In some embodiments, the protein polyurethane alloy can have a tensile strength ranging from about 10 MPa to about 18 MPa, including subranges. For example, in some embodiments, the protein polyurethane alloy can have a tensile strength ranging from about 10 MPa to about 18 MPa, about 12 MPa to about 18 MPa, or about 14 MPa to about 18 MPa, or within a range having any two of these values as endpoints, inclusive of the endpoints.

In some embodiments, the protein polyurethane alloy can have a Young's modulus ranging from about 80 MPa to about 155 MPa, including subranges. For example, in some embodiments, the protein polyurethane alloy can have a Young's modulus ranging from about 80 MPa to about 155 MPa, about 80 MPa to about 125 MPa, or about 80 MPa to about 100 MPa, or within a range having any two of these values as endpoints, inclusive of the endpoints.

In some embodiments, the protein polyurethane alloy can comprise a polyurethane having a moisture vapor transmission rate in the absence of protein. That same protein polyurethane alloy can comprise a moisture vapor transmission rate ranging from about 200 g/m²/24 hr to about 300 g/m²/24 hr greater than the moisture vapor transmission rate of the polyurethane in the absence of protein. This relative increase in moisture vapor transmission rate can be referred to as “Delta MVTR.”

In some embodiments, the protein polyurethane alloy can comprise a moisture vapor transmission rate ranging from about 500 g/m²/24 hr to about 1000 g/m²/24 hr, including subranges. For example, in some embodiments, the protein polyurethane alloy can comprise a moisture vapor transmission rate ranging from about 500 g/m²/24 hr to about 1000 g/m²/24 hr, about 500 g/m²/24 hr to about 900 g/m²/24 hr, about 500 g/m²/24 hr to about 800 g/m²/24 hr, about 500 g/m²/24 hr to about 700 g/m²/24 hr, or about 500 g/m²/24 hr to about 600 g/m²/24 hr, or within a range having any two of these values as endpoints, inclusive of the endpoints.

In some embodiments, a method of making a protein polyurethane alloy as described herein can comprise succinylating a protein, blending the succinylated protein with one or more polyurethanes in an aqueous solution to form a blended mixture comprising the succinylated protein dissolved within the one or more polyurethanes, and removing solvent from the aqueous solution.

In some embodiments, succinylating the protein can comprise adding one or more activated carboxylic acids to a protein aqueous solution comprising the protein.

Exemplary activated carboxylic acids include, but are not limited to, acyl chlorides and carboxylic acid anhydrides. In some embodiments, the activated carboxylic acid can be succinic anhydride. In other embodiments, the activated carboxylic acid can be an alkyl anhydride, such as acetic anhydride or propionic anhydride, or a succinic anhydride derivative, such as, but not limited to, maleic anhydride, methyl maleic anhydride, or itaconic anhydride.

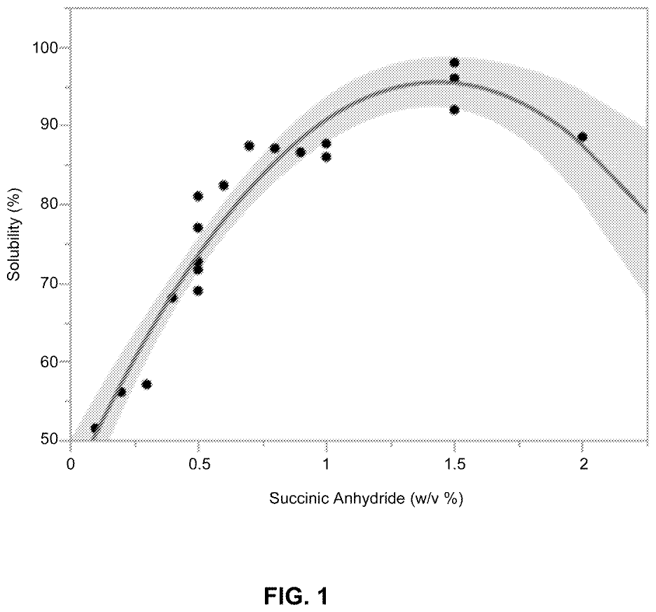

In some embodiments, the concentration of the activated carboxylic acid (measured in percent weight of activated carboxylic acid (in grams) to volume of total succinylation solution (in milliliters)) used for the succinylation reaction can be selected to achieve a desired solubility of a succinylated protein in water. For example, in some embodiments, the concentration of succinic anhydride used for a succinylation reaction can be tailored to achieve a desired solubility of a protein in water. FIG. 1 shows a graph depicting percent solubility of succinylated soy protein isolates according to some embodiments versus the succinic anhydride concentration for the succinylation reaction used to succinylate the soy protein isolates. As shown in FIG. 1, the solubility of the succinylated soy protein isolate varies across a succinic anhydride concentration of 0.1 w/v % to 2 w/v %. The solubility was about 50% at a succinic anhydride concentration of 0.1 w/v % and increased to near 100% at a succinic anhydride concentration of about 1.5 w/v %.

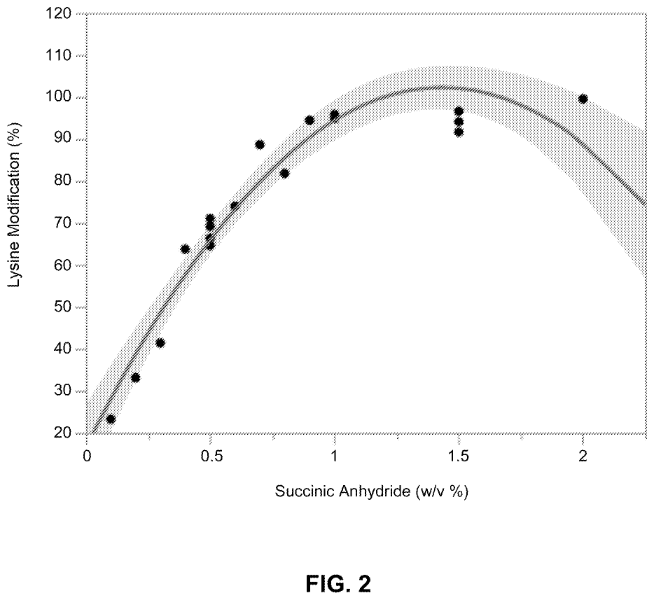

In some embodiments, the concentration of the activated carboxylic acid used for a succinylation reaction can be tailored to achieve a desired average lysine modification for a succinylated protein. For example, in some embodiments, the concentration of succinic anhydride used for the succinylation reaction can be tailored to achieve a desired average lysine modification. FIG. 2 shows a graph depicting percent solubility of succinylated soy protein isolates according to some embodiments versus the succinic anhydride concentration for the succinylation reaction used to succinylate the soy protein isolates. As shown in FIG. 2, the average lysine modification for the soy protein isolate varies across a succinic anhydride concentration of 0.1 w/v % to 2 w/v %. The average lysine modification was about 20% at a succinic anhydride concentration of 0.1 w/v % and increased to near 100% at a succinic anhydride concentration of about 1.5 w/v %.

In some embodiments, for succinylation, the concentration of the activated carboxylic acid in the protein aqueous solution can range from about 0.1 w/v % to about 15 w/v %, including subranges. For example, in some embodiments, the concentration of the activated carboxylic acid in the protein aqueous solution can range from about 0.1 w/v % to about 15 w/v %, about 0.1 w/v % to about 10 w/v %, about 0.1 w/v % to about 5 w/v %, about 0.1 w/v % to about 2 w/v %, about 0.1 w/v % to about 1.5 w/v %, about 0.1 w/v % to about 1 w/v %, or about 0.1 w/v % to about 0.5 w/v %, or within a range having any two of these values as endpoints, inclusive of the endpoints. In some embodiments, the pH of the protein aqueous solution can be above 7 and within a range of about 7.5 to about 9.5 during succinylation. In some embodiments, the pH of the protein aqueous solution can be above 7 and within a range of about 7 to about 7.5 during succinylation. In some embodiments, the pH of the protein aqueous solution can be about 8 to about 8.5 during succinylation. In some embodiments, the pH of the protein aqueous solution can be about 9 to about 9.5 during succinylation.

In some embodiments, after succinylation, the succinylated protein aqueous solution can be purified. Exemplary purification techniques include dialysis, diafiltration, and desalting chromatography.

After succinylation, the succinylated protein aqueous solution can be dried. In some embodiments, drying the succinylated protein aqueous solution can include drying the solution and forming the dried solution into a powder. Exemplary drying techniques include, but are not limited to, lyophilization and spray drying. In some embodiments, drying the succinylated protein aqueous solution can be performed after purifying the succinylated protein aqueous solution.

In some embodiments, a method of making a material comprising a protein polyurethane alloy as described herein can comprise making a layered material comprising one or more protein polyurethane alloy layers,

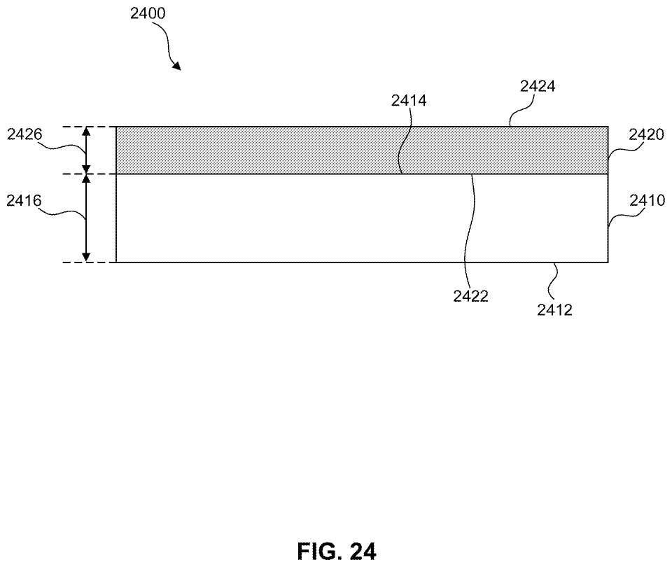

FIG. 24 shows a layered material 2400 according to some embodiments. Layered material 2400 includes a polyurethane protein alloy layer 2420 attached to a substrate layer 2410. Polyurethane protein alloy layer 2420 can be directly attached to a surface of substrate layer 2410 or attached to a surface of substrate layer 2410 via an intermediate layer, for example an adhesive layer. Direct attachment can be achieved using, for example, a thermal bonding process or a stitching. Polyurethane protein alloy layer 2420 can be referred to as a “first polyurethane protein alloy layer.”

Polyurethane protein alloy layer 2420 can include one or more types of protein and one or more polyurethanes. In some embodiments, polyurethane protein alloy layer 2420 can include one or more proteins dissolved within one or more polyurethanes. In some embodiments, polyurethane protein alloy layer 2420 can be transparent. The transparency of a polyurethane protein alloy layer is evaluated before dying or otherwise coloring a polyurethane protein alloy layer.

A transparent protein polyurethane alloy layer can provide unique characteristics for a layered material. For example, compared to a non-transparent layer, a transparent protein polyurethane alloy layer can provide unique depth of color when dyed. Likewise, a transparent protein polyurethane alloy layer can provide its mechanical properties to a layered material without significantly influencing the aesthetic properties of the material.

Protein polyurethane alloy layer 2420 includes a bottom surface 2422, a top surface 2424, and thickness 2426 measured between bottom surface 2422 and top surface 2424. In some embodiments, thickness 2426 can range from about 25 microns to about 400 microns (micrometers, μm), including subranges. For example, thickness 2426 can be about 25 microns, about 50 microns, about 100 microns, about 125 microns, about 150 microns, about 175 microns, about 200 microns, about 250 microns, about 300 microns, about 350 microns, or about 400 microns, or within a range having any two of these values as endpoints, inclusive of the endpoints. In some embodiments, thickness 2426 can range from about 50 microns to about 350 microns, about 75 microns to about 300 microns, about 100 microns to about 250 microns, about 125 microns to about 200 microns, or about 150 microns to about 175 microns.

Protein polyurethane alloy layer 2420 can have a dry weight, measured in grams per square meter (gsm, g/m²), ranging from about 25 g/m² to about 125 g/m², including subranges. For example, protein polyurethane alloy layer 2420 can have a dry weight of about 25 g/m², about 50 g/m², about 75 g/m², about 100 g/m², or about 125 g/m², or within a range having any two of these values as endpoints, inclusive of the endpoints. In some embodiments, protein polyurethane alloy layer 2420 can have a dry weight ranging from about 25 g/m² to about 125 g/m², about 25 g/m² to about 100 g/m², or about 50 g/m² to about 100 g/m².

Unless specified otherwise, the dry weight of a layer is measured during the process of making a material using the following method. First, before applying the layer in question to the material, a first sample (about 10 centimeters in diameter) of the material is cut, and the weight and dimensions are measured to calculate a first dry weight. If a sacrificial layer is present, it is removed before measuring the weight and dimensions. Second, after applying and drying the layer in question, a second sample of the same size is cut from the material, and the weight and dimensions are measured to calculate a second dry weight. If a sacrificial layer is present, it is removed before measuring the weight and dimensions. Third, the first dry weight is subtracted from the second dry weight to obtain the dry weight of the layer in question. All the weight and dimension measurements are performed at the same humidity level, typically the humidity level of the manufacturing environment in which the material is made. For purposes of calculating a dry weight, three separate dry weight tests are performed, and the average dry weight is reported as the dry weight of the layer.

In some embodiments, protein polyurethane alloy layer 2420 can be a non-foamed layer. A “non-foamed” layer means a layer having a density, measured in the percent void space for the layer, of 5% void space or less, for example 0% void space to 5% void space. In some embodiments, protein polyurethane alloy layer 2420 can be a foamed layer. In such embodiments, protein polyurethane alloy layer 2420 can have a density, measured in the percent void space for layer 2420, ranging from about 5% void space to about 70% void space, including subranges. For example, protein polyurethane alloy layer 2420 can have about 5% void space, about 10% void space, about 20% void space, about 30% void space, about 35% void space, about 40% void space, about 45% void space, about 50% void space, about 55% void space, about 60% void space, about 65% void space, or about 70% void space, or within a range having any two of these values as endpoints, inclusive of the endpoints. In some embodiments, protein polyurethane alloy layer 2420 can have a percent void space ranging from about 10% to about 65%, about 20% to about 60%, about 30% to about 55%, about 35% to about 50%, or about 40% to about 45%.

A percent void space (which can also be referred to as a “percent porosity”) can be measured by image analysis of a cross-section of a layer or by measuring the bulk density of sample of a layer using a pycnometer. Unless specified otherwise, a percent void space reported herein is measured by image analysis of a cross-section of a layer. The images are analyzed using ImageJ software (or equivalent software) at 37× magnification. The ImageJ software uses a trainable Weka segmentation classifier to calculate the percent void space in the layer. For purposes of calculating a percent void space, three to five separate images of a cross-section are evaluated, and the average percent void space is reported as the percent void space for the layer. In some embodiments, protein polyurethane alloy layer 2420 can include one or more foaming agents and/or foam stabilizers. Suitable foaming agents and foam stabilizers include those discussed herein for layers 2430 and 2440.

Substrate layer 2410 includes a bottom surface 2412, a top surface 2414, and a thickness 2416 measured between bottom surface 2412 and top surface 2414. In some embodiments, thickness 2416 can range from about 50 microns to about 1000 microns, including subranges. For example, thickness 2416 can be about 50 microns, about 100 microns, about 150 microns, about 200 microns, about 250 microns, about 300 microns, about 350 microns, about 400 microns, about 500 microns, about 600 microns, about 700 microns, about 800 microns, about 900 microns, or about 1000 microns, or within a range having any two of these values as endpoints, inclusive of the endpoints. In some embodiments, thickness 2416 can range from about 100 microns to about 900 microns, about 150 microns to about 800 microns, about 200 microns to about 700 microns, about 250 microns to about 600 microns, about 300 microns to about 500 microns, or about 350 microns to about 400 microns.

Substrate layer 2410 can have a dry weight, measured in grams per square meter (g/m²), ranging from about 50 g/m² to about 600 g/m², including subranges. For example, substrate layer 2410 can have a dry weight of about 50 g/m², about 75 g/m², about 100 g/m², about 125 g/m², about 150 g/m², about 175 g/m², about 200 g/m², about 300 g/m², about 400 g/m², about 500 g/m², or about 600 g/m², or within a range having any two of these values as endpoints. In some embodiments, substrate layer 2410 can have a dry weight ranging from about 75 g/m² to about 500 g/m², about 100 g/m² to about 400 g/m², about 125 g/m² to about 300 g/m², about 150 g/m² to about 200 g/m², or about 175 g/m² to about 200 g/m².

Substrate layer 2410 can include one or more textile layers. The one or more textile layers can be, for example, a woven layer, a non-woven layer, a knit layer, a mesh fabric layer, or a leather layer. The one or more textile layer can be comprised of recycled or virgin fibers, filaments or yarns. In some embodiments, substrate layer 2410 can be, or can include, a polyester knit layer, a polyester cotton spandex blend knit layer, or a suede layer. In some embodiments, substrate layer 2410 can be made from one or more natural fibers, for example fibers made from cotton, linen, silk, wool, kenaf, flax, cashmere, angora, bamboo, bast, hemp, soya, seacell, milk or milk proteins, spider silk, chitosan, mycelium, cellulose including bacterial cellulose, or wood. Mycelium is the vegetative part of a fungus or fungus-like bacterial colony, composed of a mass of branching, thread-like hyphae. Fungi are composed primarily of a cell wall that is constantly being extended at the apex of the hyphae. Unlike the cell wall of a plant, which is composed primarily of cellulose, or the structural component of an animal cell, which relies on collagen, the structural oligosaccharides of the cell wall of fungi rely primarily on chitin and beta glucan. Chitin is a strong, hard substance, also found in the exoskeletons of arthropods.

In some embodiments, substrate layer 2410 can be made from one or more synthetic fibers, for example fibers made from polyesters, nylons, aromatic polyamides, polyolefin fibers such as polyethylene, polypropylene, rayon, lyocell, viscose, antimicrobial yarn (A.M.Y.), Sorbtek, nylon, elastomers such as LYCRA®, spandex, or ELASTANER, polyester-polyurethane copolymers, aramids, carbon including carbon fibers and fullerenes, glass, silicon, minerals, metals or metal alloys including those containing iron, steel, lead, gold, silver, platinum, copper, zinc, and titanium, or mixtures thereof.

In some embodiments, non-woven substrate layer 2410 can be a staple non-woven, melt-blown non-woven, spunlaid non-woven, flashspun non-woven, or a combination thereof. In some embodiments, non-woven substrate layer 2410 can be made by carding, can be air-laid, or can be wet-laid. In some embodiments, the carded, air-laid, or wet-laid substrates can be bonded by, for example, needle-punch, hydroentanglement, lamination, or thermal bonding. In some embodiments, non-woven substrate layer 2410 can include one or more natural fibers, for example fibers made from cotton, linen, silk, wool, kenaf, flax, cashmere, angora, bamboo, bast, hemp, soya, seacell, milk or milk proteins, spider silk, chitosan, mycelium, cellulose including bacterial cellulose, or wood.

In some embodiments, non-woven substrate layer 2410 can include polymeric fibers with functional particles in the polymer. Exemplary functional particles include ceramic particles mixed in a polymeric resin during an extrusion process for making the polymeric fibers. Such ceramic particles can provide the polymeric fibers with desirable heat dissipation and flame resistance properties. In some embodiments, non-woven substrate layer 2410 can include fibers made of fruit pulp (e.g., grape pulp or apple pulp) or pineapple fibers. In some embodiments, non-woven substrate layer 2410 can include fibers made from recycled materials, for example recycled plastics. In some embodiments, non-woven substrate layer 2410 can include algae fibers. In some embodiments, a non-woven substrate layer 2410 can include cork fibers.

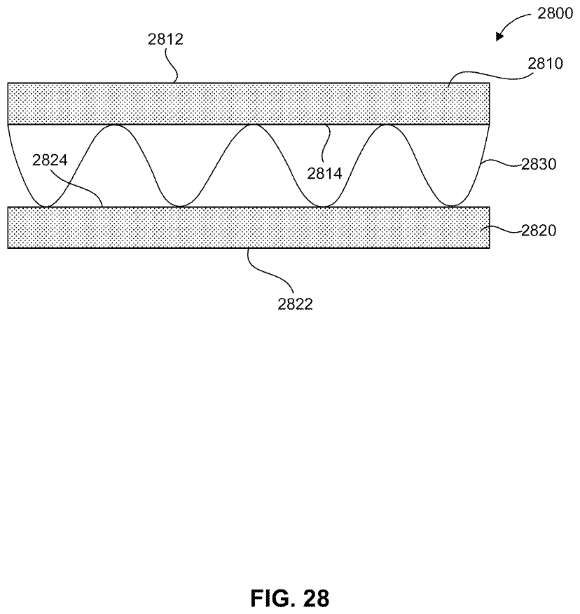

In some embodiments, substrate layer 2410 can be, or can include, a spacer fabric, for example spacer fabric 2800, shown in FIG. 28. Spacer fabric 2800 includes a first fabric layer 2810 and a second fabric layer 2820 connected by one or more spacer yarns 2830. Spacer yarn(s) 2830 are disposed between first fabric layer 2810 and second fabric layer 2820 and define a distance between an interior surface 2814 of first fabric layer 2810 and an interior surface 2824 of second fabric layer 2820. Exterior surface 2812 of first fabric layer 2810 and exterior surface 2822 of second fabric layer 2820 can define top surface 2414 and bottom surface 2412 of substrate layer 2410, respectively.

First fabric layer 2810 and second fabric layer 2820 can include one or more layers of fabric material. In some embodiments, first fabric layer 2810 and second fabric layer 2820 can include one or more textile layers made from staple fibers, filaments, or mixtures thereof. As used herein, “staple fibers” are fibers having a short length, between about 0.2 mm to about 5 centimeters (cm). Staple fibers can be naturally occurring or can be cut filaments. As used herein, “filaments” are long fibers having a length of 5 cm or more. In some embodiments, first fabric layer 2810 and second fabric layer 2820 can include one or more layers of a woven material or a knitted material. In some embodiments, exterior surface 2812 of first fabric layer 2810 can be defined by a woven fabric layer or a knitted fabric layer. In some embodiments, exterior surface 2822 of second fabric layer 2820 can be defined by a woven fabric layer or a knitted fabric layer.

In some embodiments, first fabric layer 2810 and second fabric layer 2820 can be made from one or more natural fibers, for example fibers made from cotton, linen, silk, wool, kenaf, flax, cashmere, angora, bamboo, bast, hemp, soya, seacell, milk or milk proteins, spider silk, chitosan, mycelium, cellulose including bacterial cellulose, or wood. In some embodiments, first fabric layer 2810 and second fabric layer 2820 can be made from one or more synthetic fibers, for example fibers made from polyesters, nylons, aromatic polyamides, polyolefin fibers such as polyethylene, polypropylene, rayon, lyocell, viscose, antimicrobial yarn (A.M.Y.), Sorbtek, nylon, elastomers such as LYCRA R, spandex, or ELASTANER, polyester-polyurethane copolymers, aramids, carbon including carbon fibers and fullerenes, glass, silicon, minerals, metals or metal alloys including those containing iron, steel, lead, gold, silver, platinum, copper, zinc, and titanium, or mixtures thereof. Spacer yarn(s) 2830 can include mono-filament yarn(s) composed of any of the natural or synthetic materials listed above for first fabric layer 2810 and second fabric layer 2820.

In some embodiments, substrate layer 2410 can be colored with a coloring agent. In some embodiments the coloring agent can be a dye, for example an acid dye, a fiber reactive dye, a direct dye, a sulfur dye, a basic dye, or a reactive dye. In some embodiments, the coloring agent can be pigment, for example a lake pigment. In some embodiments, a first coloring agent can be incorporated into one or more protein polyurethane alloy layers and a second coloring agent can be incorporated into substrate layer 2410, depending on the desired aesthetic of a layered material.

A fiber reactive dye includes one or more chromophores that contain pendant groups capable of forming covalent bonds with nucleophilic sites in fibrous, cellulosic substrates in the presence of an alkaline pH and raised temperature. These dyes can achieve high wash fastness and a wide range of brilliant shades. Exemplary fiber reactive dyes, include but are not limited to, sulphatoethylsulphone (Remazol), triazine, vinylsulphone, and acrylamido dyes. These dyes can dye protein fibers such as silk, wool and nylon by reacting with fiber nucleophiles via a Michael addition. Direct dyes are anionic dyes capable of dying cellulosic or protein fibers. In the presence of an electrolyte such as sodium chloride or sodium sulfate, near boiling point, these dyes can have an affinity to cellulose. Exemplary direct dyes include, but are not limited to, azo, stilbene, phthalocyanine, and dioxazine.

In some embodiments, layered material 2400 can include a protein polyurethane alloy layer 2420 attached to top surface 2414 of substrate layer 2410. In some embodiments, bottom surface 2422 of protein polyurethane alloy layer 2420 can be in direct contact with top surface 2414 of substrate layer 2410. In some embodiments, bottom surface 2422 of protein polyurethane alloy layer 2420 can be attached to top surface 2414 of substrate layer 2410 via an adhesive layer (e.g., adhesive layer 2450). In some embodiments, layered material 2400 can include a protein polyurethane alloy layer 2420 attached to bottom surface 2412 of substrate layer 2410. In some embodiments, top surface 2424 of protein polyurethane alloy layer 2420 can be in direct contact with bottom surface 2412 of substrate layer 2410. In some embodiments, top surface 2424 of protein polyurethane alloy layer 2420 can be attached to bottom surface 2412 of substrate layer 2410 via an adhesive layer (e.g., adhesive layer 2450). In some embodiments, layered material 2400 can include a protein polyurethane alloy layer 2420 attached to top surface 2414 of substrate layer 2410 and a protein polyurethane alloy layer 2420 attached to bottom surface 2412 of substrate layer 2410. In such embodiments, layered material 2400 includes protein polyurethane alloy layers 2420 disposed on opposing surfaces of substrate layer 2410.

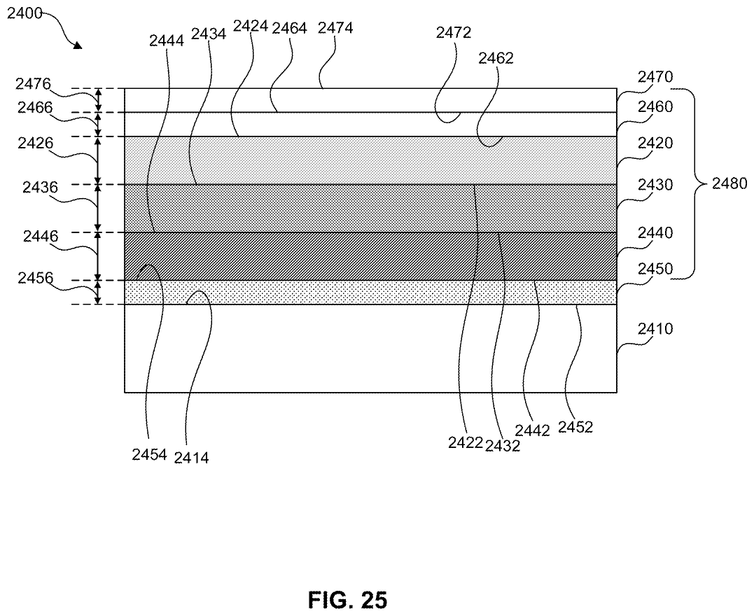

In some embodiments, as shown for example in FIG. 25, layered material 2400 can include a second protein polyurethane alloy layer 2430 disposed between protein polyurethane alloy layer 2420 and substrate layer 2410. In such embodiments, second protein polyurethane alloy layer 2430 is attached to protein polyurethane alloy layer 2420. In some embodiments, bottom surface 2422 of protein polyurethane alloy layer 2420 can be in direct contact with a top surface 2434 of second protein polyurethane alloy layer 2430.

Second protein polyurethane alloy layer 2430 includes a bottom surface 2432, top surface 2434, and a thickness 2436 measured between bottom surface 2432 and top surface 2434. In some embodiments, thickness 2436 can range from about 25 microns to about 600 microns, including subranges. For example, thickness 2436 can be about 25 microns, about 50 microns, about 100 microns, about 125 microns, about 150 microns, about 175 microns, about 200 microns, about 225 microns, about 250 microns, about 275 microns, about 300 microns, about 400 microns, about 500 microns, or about 600 microns, or within a range having any two of these values as endpoints, inclusive of the endpoints. In some embodiments, thickness 2436 can range from about 50 microns to about 500 microns, about 75 microns to about 400 microns, about 100 microns to about 300 microns, about 125 microns to about 275 microns, about 150 microns to about 250 microns, about 175 microns to about 225 microns, or about 200 microns to about 225 microns. In some embodiments, thickness 2436 can be greater than thickness 2426. In some embodiments, thickness 2436 can be less than thickness 2426. In some embodiments, thickness 2436 can be greater than or less than thickness 2426 by 5 microns or more.

Second protein polyurethane alloy layer 2430 can have a dry weight, measured in grams per square meter (g/m²), ranging from about 30 g/m² to about 600 g/m², including subranges. For example, second protein polyurethane alloy layer 2430 can have a dry weight of about 30 g/m², about 40 g/m², about 60 g/m², about 80 g/m², about 100 g/m², about 120 g/m², about 140 g/m², about 150 g/m², about 200 g/m², about 300 g/m², about 400 g/m², about 500 g/m², or about 600 g/m², or within a range having any two of these values as endpoints, inclusive of the endpoints. In some embodiments, second protein polyurethane alloy layer 2430 can have a dry weight ranging from about 40 g/m² to about 500 g/m², about 60 g/m² to about 400 g/m², about 80 g/m² to about 300 g/m², about 100 g/m² to about 200 g/m², about 120 g/m² to about 150 g/m², or about 140 g/m² to about 150 g/m². In some embodiments, protein polyurethane alloy layer 2420 can have a first weight and second protein polyurethane alloy layer 2430 can have a second weight, and the first weight can be less than the second weight. In some embodiments, the first weight can be less than the second weight by 5 g/m² or more.

In some embodiments, second protein polyurethane alloy layer 2430 can include a foaming agent. In some embodiments, second protein polyurethane alloy layer 2430 can include a foam stabilizer. The foaming agent or foam stabilizer can facilitate the formation of voids in second protein polyurethane alloy layer 2430 during blending of second protein polyurethane alloy layer 2430. Suitable foam stabilizers include, but are not limited to, HeiQ Chemtex 2216-T (a stabilized blend of nonionic and anionic surfactants), HeiQ Chemtex 2241-A (a modified HEUR (hydrophobically-modified ethylene oxide urethane) thickener), HeiQ Chemtex 2243 (a non-ionic silicone dispersion), or HeiQ Chemtex 2317 (a stabilized blend of nonionic and anionic surfactants) foam stabilizers available from HeiQ Chemtex. When used, a foam stabilizer serves to stabilize mechanically created foam (air voids). The mechanically created foam may be created by, for example, a rotor and/or compressed air. When used, a foaming agent can create foam (air voids) within a layer by a chemical reaction and/or via heat generation within the layer.

In some embodiments, second protein polyurethane alloy layer 2430 can be referred to as a “foamed protein polyurethane alloy layer” because either (i) layer 2430 includes one or more foaming agents or foam stabilizers and/or (ii) layer 2430 includes a density less than protein polyurethane alloy layer 2420.

Second protein polyurethane alloy layer 2430 can have a density, measured in the percent void space for layer 2430, ranging from about 5% void space to about 70% void space, including subranges. For example, second protein polyurethane alloy layer 2430 can have about 5% void space, about 10% void space, about 20% void space, about 30% void space, about 35% void space, about 40% void space, about 45% void space, about 50% void space, about 55% void space, about 60% void space, about 65% void space, or about 70% void space, or within a range having any two of these values as endpoints, inclusive of the endpoints. In some embodiments, second protein polyurethane alloy layer 2430 can have a percent void space ranging from about 10% to about 65%, about 20% to about 60%, about 30% to about 55%, about 35%, to about 50%, or about 40% to about 45%. In some embodiments, protein polyurethane alloy layer 2420 can have a first density and second protein polyurethane alloy layer 2430 can have a second density, and the first density can be greater than the second density. In some embodiments, the first density can be greater than the second density by 5% void space or more.

Layering a plurality of protein polyurethane alloy layers having different weights and/or densities can be used to tailor the material properties of a layered material. For example, layers having lighter weights and/or densities can be used to increase the softness and/or flexibility of a layered material. On the other hand, layers having high weights and/or densities can increase the strength of the layered material. Additionally, using one or more layers having relatively lighter weight and/or density can increase the ease in which cutting, stitching, and/or shaping process steps (e.g., skyving) can be performed on a layered material. Layering a plurality of protein polyurethane alloy layers gives lot of freedom in designing of a material.

In some embodiments, second protein polyurethane alloy layer 2430 can further include, in addition to any other components that may be present, such as a foaming agent, a foam stabilizer, or one or more coloring agents. The coloring agent type and content for second protein polyurethane alloy layer 2430 can be any of the types and amounts described herein for protein polyurethane alloy layer 2420. In some embodiments, second protein polyurethane alloy layer 2430 can be free or substantially free of a coloring agent.

In some embodiments, as shown for example in FIG. 25, layered material 2400 can include a third protein polyurethane alloy layer 2440 disposed between second protein polyurethane alloy layer 2430 and substrate layer 2410. In such embodiments, third protein polyurethane alloy layer 2440 is attached to second protein polyurethane alloy layer 2430. In some embodiments, bottom surface 2432 of second protein polyurethane alloy layer 2430 can be in direct contact with a top surface 2444 of third protein polyurethane alloy layer 2440.

Third protein polyurethane alloy layer 2440 includes a bottom surface 2442, top surface 2444, and a thickness 2446 measured between bottom surface 2442 and top surface 2444. In some embodiments, thickness 2446 can range from about 25 microns to about 600 microns, including subranges. For example, thickness 2446 can be about 25 microns, about 50 microns, about 100 microns, about 125 microns, about 150 microns, about 175 microns, about 200 microns, about 225 microns, about 250 microns, about 275 microns, about 300 microns, about 400 microns, about 500 microns, or about 600 microns, or within a range having any two of these values as endpoints, inclusive of the endpoints. In some embodiments, thickness 1746 can range from about 50 microns to about 500 microns, about 75 microns to about 400 microns, about 100 microns to about 300 microns, about 125 microns to about 275 microns, about 150 microns to about 250 microns, about 175 microns to about 225 microns, or about 175 microns to about 200 microns. In some embodiments, thickness 2446 can be greater than thickness 2426. In some embodiments, thickness 2446 can be less than thickness 2426. In some embodiments, thickness 2446 can be greater than or less than thickness 2426 by 5 microns or more. In some embodiments, thickness 2446 can be the same as thickness 2436. In some embodiments, thickness 2446 can be greater than or less than thickness 2436. In some embodiments, thickness 2446 can be greater than or less than thickness 2436 by 5 microns or more.

Third protein polyurethane alloy layer 2440 can have a dry weight, measured in grams per square meter (g/m²), ranging from about 30 g/m² to about 600 g/m², including subranges. For example, third protein polyurethane alloy layer 2440 can have a dry weight of about 30 g/m², about 40 g/m², about 60 g/m², about 80 g/m², about 100 g/m², about 120 g/m², about 140 g/m², about 150 g/m², about 200 g/m², about 300 g/m², about 400 g/m², about 500 g/m², or about 600 g/m², or within a range having any two of these values as endpoints, inclusive of the endpoints. In some embodiments, third protein polyurethane alloy layer 2440 can have a dry weight ranging from about 40 g/m² to about 500 g/m², about 60 g/m² to about 400 g/m², about 80 g/m² to about 300 g/m², about 100 g/m² to about 200 g/m², about 120 g/m² to about 150 g/m², or about 120 g/m² to about 140 g/m². In some embodiments, protein polyurethane alloy layer 2420 can have a first weight and third protein polyurethane alloy layer 2440 can have a third weight, and the first weight can be less than the third weight. In some embodiments, protein polyurethane alloy layer 2420 can have a first weight, second protein polyurethane alloy layer 2430 can have a second weight, and third protein polyurethane alloy layer 2440 can have a third weight, and the first weight can be less than the second weight and the third weight. In some embodiments, the first weight can be less than the second weight and/or the third weight by 5 g/m² or more.

In some embodiments, third protein polyurethane alloy layer 2440 can include a foaming agent. In some embodiments, third protein polyurethane alloy layer 2440 can include a foam stabilizer. The foaming agent and/or foam stabilizer can facilitate the formation of voids in third protein polyurethane alloy layer 2440 during blending of third protein polyurethane alloy layer 2440. Suitable foaming agents include, but are not limited to, HeiQ Chemtex 2216-T (a stabilized blend of nonionic and anionic surfactants), HeiQ Chemtex 2241-A (a modified HEUR (hydrophobically-modified ethylene oxide urethane) thickener), HeiQ Chemtex 2243 (a non-ionic silicone dispersion), or HeiQ Chemtex 2317 (a stabilized blend of nonionic and anionic surfactants) foam stabilizers available from HeiQ Chemtex.

In some embodiments, third protein polyurethane alloy layer 2440 can be referred to as a “foamed protein polyurethane alloy layer” because either (i) layer 2440 includes one or more foaming agents or foam stabilizers and/or (ii) layer 2440 includes a density less than protein polyurethane alloy layer 2420.

Third protein polyurethane alloy layer 2440 can have a density, measured in the percent void space for layer 2440, ranging from about 5% void space to about 70% void space, including subranges. For example, third protein polyurethane alloy layer 2440 can have about 5% void space, about 10% void space, about 20% void space, about 30% void space, about 35% void space, about 40% void space, about 45% void space, about 50% void space, about 55% void space, about 60% void space, about 65% void space, or about 70% void space, or within a range having any two of these values as endpoints, inclusive of the endpoints. In some embodiments, third protein polyurethane alloy layer 2440 can have a percent void space ranging from about 10% to about 65%, about 20% to about 60%, about 30% to about 55%, about 35% to about 50%, or about 40% to about 45%. In some embodiments, protein polyurethane alloy layer 2420 can have a first density and third protein polyurethane alloy layer 2440 can have a third density, and the first density can be greater than the third density. In some embodiments, protein polyurethane alloy layer 2420 can have a first density, second protein polyurethane alloy layer 2430 can have a second density, and third protein polyurethane alloy layer 2440 can have a third density, and the first density can be greater than the second density and third density. In some embodiments, the first density can be greater than the second density and/or the third density by 5% void space or more.

In some embodiments, layered material 2400 can include a plurality of protein polyurethane alloy layers having the same protein and polyurethane. In some embodiments, layered material 2400 can include a plurality of protein polyurethane alloy layers and the different layers can have a different protein and/or a different polyurethane.

In some embodiments, third protein polyurethane alloy layer 2440 can further include, in addition to any other components that may be present, such as a foaming agent, a foam stabilizer, one or more coloring agents. The coloring agent type and content for third protein polyurethane alloy layer 2440 can be any of the types and amounts described herein for protein polyurethane alloy layer 2420. In some embodiments, third protein polyurethane alloy layer 2440 can be free or substantially free of a coloring agent.

In some embodiments, and as shown for example in FIG. 25, layered material 2400 can include a basecoat layer 2460. Basecoat layer 2460 can be disposed over top surface 2424 of protein polyurethane alloy layer 2420. Basecoat layer 2460 can be directly or indirectly attached to protein polyurethane alloy layer 2420. In some embodiments, basecoat layer 2460 can be disposed on top surface 2424 of protein polyurethane alloy layer 2420. In some embodiments, a bottom surface 2462 of basecoat layer 2460 can be in direct contact with top surface 2424 of protein polyurethane alloy layer 2420.

Basecoat layer 2460 includes bottom surface 2462, a top surface 2464, and a thickness 2466 measured between bottom surface 2462 and top surface 2464. In some embodiments, thickness 2466 can range from about 20 microns to about 200 microns, including subranges. For example, thickness 2466 can be about 20 microns, about 30 microns, about 40 microns, about 50 microns, about 60 microns, about 70 microns, about 80 microns, about 90 microns, about 100 microns, about 150 microns, or about 200 microns, or within a range having any two of these values as endpoints, inclusive of the endpoints. In some embodiments, thickness 2466 can range from about 30 microns to about 150 microns, about 40 microns to about 100 microns, about 50 microns to about 90 microns, about 60 microns to about 80 microns, or about 60 microns to about 70 microns.

In embodiments including basecoat layer 2460, basecoat layer 2460 can provide one or more of the following properties for layered material 2400: (i) abrasion performance, color fastness, or hydrolytic resistance. Basecoat layer 2460 may also serve to adhere to a top-coat layer to layered material 2400 in embodiments including a top-coat layer. In some embodiments, basecoat layer 2460 can include one or more polymeric materials. Suitable materials for basecoat layer 2460 include, but are not limited to, polyether polyurethanes, polycarbonate polyurethanes, polyester polyurethanes, acrylic polymers, and cross-linkers such as isocyanate or carbodiimide. In some embodiments, layered material 2400 can include a plurality of basecoat layers 2460. In some embodiments, basecoat layer 2460 can be absent from layered material 2400.

Basecoat layer 2460 can have a dry weight, measured in grams per square meter (g/m²), ranging from about 20 g/m² to about 100 g/m², including subranges. For example, basecoat layer 2460 can have a dry weight of about 20 g/m², about 30 g/m², about 40 g/m², about 50 g/m², about 60 g/m², about 70 g/m², about 80 g/m², about 90 g/m², or about 100 g/m², or within a range having any two of these values as endpoints, inclusive of the endpoints. In some embodiments, basecoat layer 2460 can have a dry weight ranging from about 30 g/m² to about 90 g/m², about 40 g/m² to about 80 g/m², or about 50 g/m² to about 70 g/m².

In some embodiments, as shown for example in FIG. 25, layered material 2400 can include a top-coat layer 2470. Top-coat layer 2470 can be disposed over top surface 2424 of protein polyurethane alloy layer 2420. Top-coat layer 2470 can be directly or indirectly attached to protein polyurethane alloy layer 2420. In some embodiments, a bottom surface 2472 of top-coat layer 2470 can be in direct contact with top surface 2424 of protein polyurethane alloy layer 2420. In embodiments including basecoat layer 2460, top-coat layer 2470 can be disposed over top surface 2464 of basecoat layer 2460. In some embodiments, top-coat layer 2470 can be disposed on top surface 2464 of basecoat layer 2460. In some embodiments, a bottom surface 2472 of top-coat layer 2470 can be in direct contact with top surface 2464 of basecoat layer 2460.

Top-coat layer 2470 includes bottom surface 2472, a top surface 2474, and a thickness 2476 measured between bottom surface 2472 and top surface 2474. In some embodiments, thickness 2476 can range from about 10 microns to about 80 microns, including subranges. For example, thickness 2476 can be about 10 microns, about 20 microns, about 30 microns, about 40 microns, about 50 microns, about 60 microns, about 70 microns, or about 80 microns, or within a range having any two of these values as endpoints, inclusive of the endpoints. In some embodiments, thickness 2476 can range from about 20 microns to about 70 microns, about 30 microns to about 60 microns, or about 30 microns to about 50 microns.

In embodiments including top-coat layer 2470, top-coat layer 2470 can provide one or more of the following properties for layered material 2400: surface feel, stain resistance, flame resistance, gloss level, or color appearance. In some embodiments, top-coat layer 2470 can include one or more polymeric materials. Suitable materials for top-coat layer 2470 include but are not limited to, polyurethanes, acrylics, silicone-based feel agents, matte agents, and gloss agents. In some embodiments, layered material 2400 can include a plurality of top-coat layers 2470. In some embodiments, top-coat layer 2470 can be absent from layered material 2400. In some embodiments, top-coat layer 2470 can be transparent or translucent. In some embodiments, top-coat layer 2470 can include one or more dyes, one or more pigments and/or one or more reflective agents to affect appearance.

Top-coat layer 2470 can have a dry weight, measured in grams per square meter (g/m²), ranging from about 10 g/m² to about 80 g/m², including subranges. For example, top-coat layer 2470 can have a dry weight of about 10 g/m², about 20 g/m², about 30 g/m², about 40 g/m², about 50 g/m², about 60 g/m², about 70 g/m², or about 80 g/m², or within a range having any two of these values as endpoints, inclusive of the endpoints. In some embodiments, top-coat layer 2470 can have a dry weight ranging from about 20 g/m² to about 70 g/m², about 30 g/m² to about 60 g/m², or about 30 g/m² to about 50 g/m².

Together, protein polyurethane alloy layer(s) 2420, 2430, 2440, basecoat layer(s) 2460, and/or top-coat layer(s) 2470 can define a layered assembly 2480 of a layered material 2400. Layered assembly 2480 can include any number of protein polyurethane alloy layers as described herein. For example, layered assembly 2480 can include 2, 3, 4, 5, 6, 7, 8, 9, 10, 11, 12, 13, 14, 15, 16, 17, 18, 19, or 20 protein polyurethane alloy layers. In some embodiments, layered material 2400 can include a layered assembly 2480 attached to bottom surface 2412 of substrate layer 2410. Layered assembly 2480 attached to bottom surface 2412 of substrate layer 2410 can include any of the layers and materials as described herein for a layered assembly 2480 attached to top surface 2414 of substrate layer 2410. In some embodiments, layered material 2400 can include a layered assembly 2480 attached to top surface 2414 of substrate layer 2410 and a layered assembly 2480 attached to bottom surface 2412 of substrate layer 2410. In such embodiments, layered material 2400 includes layered assemblies 2480 disposed over opposing surfaces 2412 and 2414 of substrate layer 2410.

In some embodiments, a protein polyurethane alloy layer of layered material 2400 is attached to a surface of substrate layer 2410 with an adhesive layer 2450. In such embodiments, adhesive layer 2450 includes a bottom surface 2452, a top surface 2454, and a thickness 2456 measured between bottom surface 2452 and top surface 2454. In some embodiments, thickness 2456 can range from about 10 microns to about 50 microns, including subranges. For example, thickness 2456 can be about 10 microns, about 20 microns, about 30 microns, about 40 microns, or about 50 microns, or within a range having any two of these values as endpoints, inclusive of the endpoints. In some embodiments, thickness 2456 can range from about 20 microns to about 40 microns. Suitable adhesives for adhesive layer 2450 include, but are not limited to, polyurethane adhesives, hot melt adhesives, emulsion polymer adhesives, dry web adhesives, dry laminating adhesives, or wet laminating adhesives. Hauthane HD-2001 available from C.L. Hauthaway & Sons Corporation is an exemplary laminating adhesive suitable for adhesive layer 1750. Exemplary polyurethane adhesives include, but are not limited to, L-2183, L-2245, L-2255 from Hauthaway and IMPRANILR DAH, DAA from Covestro. Exemplary dry web adhesives include, but are not limited to, 9D8D20 from Protechnic. In some embodiments, layered material 2400 does not include an adhesive layer 2450.

Adhesive layer 2450 can have a dry weight, measured in grams per square meter (g/m²), ranging from about 10 g/m² to about 50 g/m², including subranges. For example, adhesive layer 2450 can have a dry weight of about 10 g/m², about 20 g/m², about 30 g/m², about 40 g/m², or about 50 g/m², or within a range having any two of these values as endpoints, inclusive of the endpoints. In some embodiments, adhesive layer 2450 can have a dry weight ranging from about 20 g/m² to about 40 g/m².

Layered material 2400 can be made by attaching one or more protein polyurethane alloy layers, and one or more basecoat and/or top-coat layers described herein, to substrate layer 2410. In some embodiments, the layer(s) may be subsequently layered over a surface of a substrate layer. Layer(s) can be attached to either top surface 2414 and/or bottom surface 2412 of substrate layer 2410. In some embodiments, the layer(s) can be layered over a sacrificial layer that is removed after layering and before or after attaching the one or more layers to substrate layer 2410. Each protein polyurethane alloy layer of a layered material can be deposited using any suitable coating technique, including, but not limited to, knife over roll coating, gravure coating, slot die coating, multi-layer slot die coating, or curtain coating. Multi-layer slot die coating can allow simultaneous coating of multiple adjacent layers.

In some embodiments, a substrate layer 2410 can be coated with an adhesive layer 2450 and additional layers (e.g., layers 2420, 2430, 2440, 2460, and/or 2470) can be formed over adhesive layer 2450 in any appropriate order. In such embodiments, the layers can be formed over adhesive layer 2450 in the same manner as described below for method 2600 with the layers being formed over the adhesive layer 2450 rather than a sacrificial layer. In some embodiments, a blended mixture as described herein can be applied directly to a surface of a substrate layer 2410, using for example, a coating or pouring process. In such embodiments, the blended mixture can penetrate at least a portion of substrate layer 2410. After application, the blended mixture can be dried to form a protein polyurethane alloy layer (e.g., layer 2420). In some embodiments, after drying, the protein polyurethane alloy layer and the substrate layer 2410 can be heated (e.g., heat pressed) to aid in attaching the layers together. Before or after drying and/or before or after attaching the protein polyurethane alloy layer and substrate layer 2410, other layers (e.g., layers 2430, 2440, 2460, and/or 2470) can be applied over the protein polyurethane alloy layer in any appropriate order. In such embodiments, the other layers can be formed over the protein polyurethane alloy layer in the same manner as described below for method 2600 with the layers being formed over the protein polyurethane alloy layer rather than a sacrificial layer.

In some embodiments, decorative layers can be applied between layers of a layered material during manufacturing. For example, a logo, an artistic pattern, a drawing, or a symbol can be applied to a first layer before disposing another layer over the first layer. Decorative layers can be applied using, for example, screen printing, digital printing, or transfer printing.

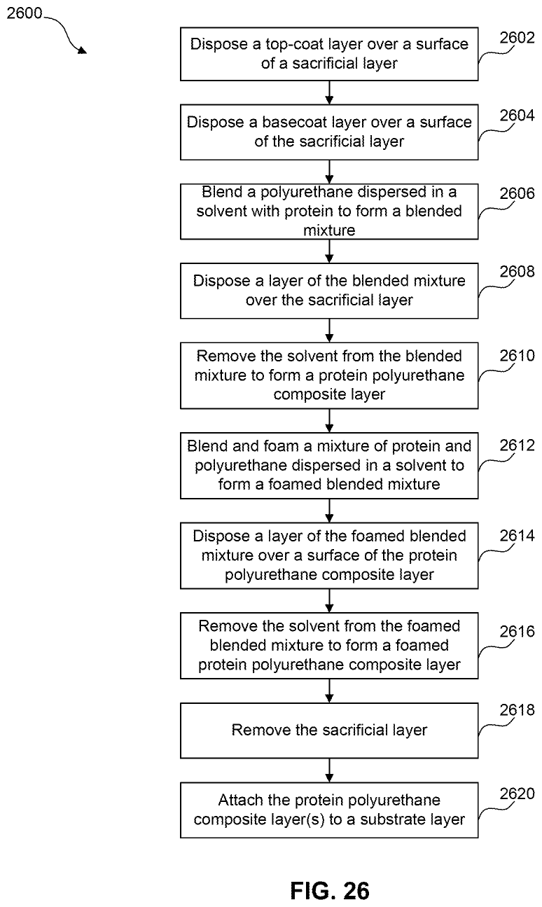

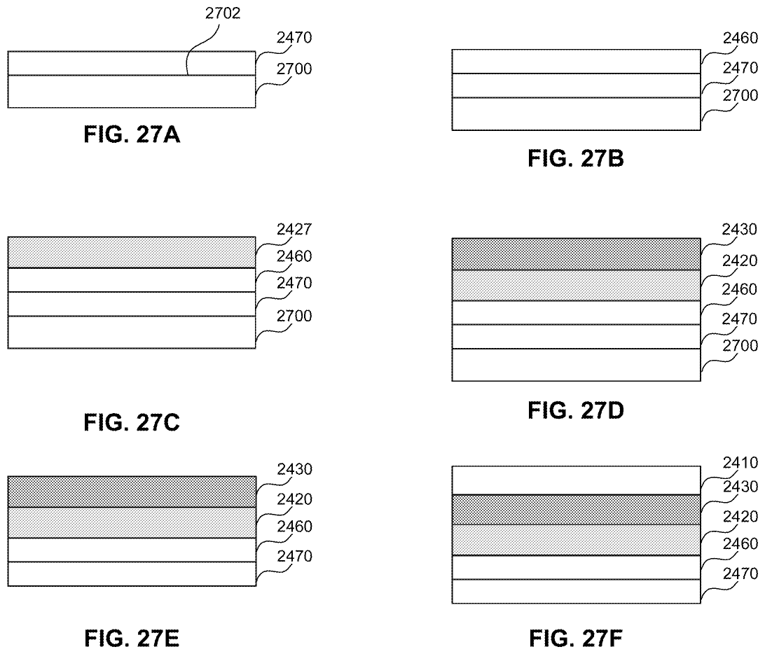

In some embodiments, the layers of a layered material can be formed over a sacrificial layer and attached to a substrate layer after formation. FIG. 26 illustrates a method 2600 for making a layered material 2400 according to some embodiments. FIGS. 27A-27F illustrate steps of method 2600. Unless stated otherwise, the steps of method 2600 need not be performed in the order set forth herein. Additionally, unless specified otherwise, the steps of method 2600 need not be performed sequentially. The steps can be performed simultaneously. As one example, method 2600 need not include a solvent removal step after the deposition of each individual protein polyurethane alloy layer; rather the solvent (for example, water) from a plurality of protein polyurethane alloy layers can be removed in a single step. Method 2600 can be used to attach layers to one or both sides of a substrate layer 2410.

In step 2602, a top-coat layer 2470 can be disposed over a top surface 2702 of a sacrificial layer 2700, as illustrated in for example FIG. 27A. Top-coat layer 2470 can be disposed over sacrificial layer 2700 using any suitable coating technique, for example, knife over roll with reverse transfer paper, spraying, or roller coating. Sacrificial layer 2700 is a layer of material that does not define a layer of layered material 2400. Rather, sacrificial layer 2700 is removed during manufacturing of layered material 2400. Sacrificial layer 2700 can be removed mechanically, such as by peeling away, or chemically, for example, by dissolving sacrificial layer 2700. In some embodiments, sacrificial layer 2700 can be a release liner. Suitable materials for sacrificial layer 2700 include but are not limited to grain texture release papers. Exemplary grain texture release papers include, release papers available from Sappi paper, for example, Matte Freeport 189, Freeport 123, or Expresso 904. In some embodiments, method 2600 does not include step 2602. That is, step 2602 is optional. In some embodiments, top-coat layer 2470 can be applied to a layered material 2400 after removing sacrificial layer 2700 in step 2618. In some embodiments, top-coat layer 2470 can be applied to a layered material 2400 after attaching protein polyurethane alloy layer(s) to a substrate layer 2410 in step 2620.

In step 2604, basecoat layer 2460 can be disposed over sacrificial layer 2700, as illustrated in for example FIG. 27B. In embodiments including top-coat layer 2470, basecoat layer 2460 can be disposed over top-coat layer 2470. Basecoat layer 2460 can disposed over sacrificial layer 2700 using any suitable coating technique, for example, knife over roll with reverse transfer paper, spraying, or roller coating. In some embodiments, method 2600 does not include step 2604. Step 2604 is optional. In some embodiments, basecoat layer 2460 can be applied to a layered material 2400 after removing sacrificial layer 2700 in step 2618. In some embodiments, basecoat layer 2460 can be applied to a layered material 2400 after attaching protein polyurethane alloy layer(s) to a substrate layer 2410 in step 2620.

In step 2606, one or more polyurethanes dispersed or dissolved in an aqueous solution can be blended with one or more proteins to form a blended mixture in the aqueous solution. In some embodiments, the one or more polyurethanes can be dispersed or dissolved in an aqueous solution before blending with protein(s). In some embodiments, the one or more polyurethanes can become dispersed or dissolved in an aqueous solution during blending with protein(s). In some embodiments, the one or more polyurethanes and the one or more proteins can be blended in a suitable vessel until a homogenous blend is formed. Suitable blending equipment includes, but is not limited to, a blender, a stand mixer, an in-line mixer, or a high shear mixer.

In some embodiments, protein(s) can be dispersed or dissolved in an aqueous solution before blending with polyurethane in step 2606. Suitable aqueous solutions include, but are not limited to, water, an aqueous alkali solution, an aqueous acid solution, an aqueous solution including an organic solvent, a urea solution, and mixtures thereof. In some embodiments, the aqueous alkali solution can be a basic solution such as a sodium hydroxide, ammonia or ammonium hydroxide solution. In some embodiments, examples of an acidic aqueous solution can be an acetic acid or hydrochloric acid (HCl) solutions. Suitable organic solvents include, but are not limited to, ethanol, isopropanol, acetone, ethyl acetate, isopropyl acetate, glycerol, and the like. In some embodiments, the protein concentration in the aqueous protein mixture can range from about 10 g/L to about 300 g/L, including subranges. For example, the protein concentration in the aqueous protein mixture can be about 10 g/L, about 20 g/L, about 30 g/L, about 40 g/L, about 50 g/L, about 60 g/L, about 70 g/L, about 80 g/L, about 90 g/L, about 100 g/L, about 150 g/L, about 200 g/L, about 250 g/L, or about 300 g/L, or within a range having any two of these values as endpoints, inclusive of the endpoints. In some embodiments, the protein concentration in the aqueous protein mixture can range from about 10 g/L to about 300 g/L, about 20 g/L to about 250 g/L, about 30 g/L to about 200 g/L, about 40 g/L to about 150 g/L, about 50 g/L to about 100 g/L, about 60 g/L to about 90 g/L, or about 70 g/L to about 80 g/L.

The amount of protein in a protein/polyurethane blend can range from about 5 wt % to about 60%, based on the weight of protein and polyurethane, including subranges. For example, the amount of protein in a blend can be about 5 wt %, about 10 wt %, about 15 wt %, about 20 wt %, about 25 wt %, about 30 wt %, about 35 wt %, about 40 wt %, about 45 wt %, about 50 wt %, about 55 wt %, or about 60 wt %, or within a range having any two of these values as endpoints. In some embodiments, the amount of protein in a blend can be about 10 wt % to about 55 wt %, about 15 wt % to about 50 wt %, about 20 wt % to about 45 wt %, about 25 wt % to about 40 wt %, or about 30 wt % to about 35 wt %. In some embodiments, the amount of protein in the protein/polyurethane blend can range from 20 wt % to 40 wt %.

The amount of polyurethane(s) in a protein/polyurethane blend can range from about 10 wt % to about 85 wt %, based on the weight of protein and polyurethane, including subranges. For example, the amount of polyurethane(s) in blend can be about 10 wt %, about 15 wt %, about 20 wt %, about 25 wt %, about 30 wt %, about 35 wt %, about 40 wt %, about 45 wt %, about 50 wt %, about 55 wt %, about 60 wt %, about 65 wt %, about 70 wt %, about 75 wt %, about 80 wt %, or about 85 wt %, or within a range having any two of these values as endpoints, inclusive of the endpoints. In some embodiments, the amount of the polyurethane(s) in a blend can range from about 20 wt % to about 75 wt %, about 30 wt % to about 65 wt %, or about 40 wt % to about 55 wt %.

In some embodiments, the blending temperature can range from about room temperature (18° C.) to about 100° C., including subranges. For example, the blend temperature can be about 18° C., about 30° C., about 40° C., about 50° C., about 60° C., about 70° C., about 80° C., about 90° C., or about 100° C., or within a range having any two of these values as endpoints, inclusive of the endpoints. In some embodiments, the blend temperature can range from about 18° C. to about 90° C., about 18° C. to about 80° C., about 18° C. to about 70° C., about 18° C. to about 60° C., about 18° C. to about 50° C., about 18° C. to about 40° C., or about 18° C. to about 30° C.

In some embodiments, the blending time for step 2606 can range from about 15 minutes to about 3 hours. In some embodiments, the blending speed for step 2606 can range from about 150 rpm to about 250 rpm. The blending speed can depend on the size of a blending device (e.g., size of an impeller) and/or the size of the vessel in which the components are blended.

In some embodiments, one or more additives can be added to the blend in step 2606. The additive(s) can influence the final properties of a protein polyurethane alloy layer, and therefore the final properties of a layered material 2400. For example, the additive(s) added can impact one or more of the following material properties: stiffness, elasticity, cohesive strength, tear strength, fire retardancy, chemical stability, or wet stability. Suitable additives include, but are not limited to, cross-linkers, fillers, dyes, pigments, plasticizers, waxes, rheological modifiers, flame retardants, antimicrobial agents, antifungal agents, antioxidants, UV stabilizers, mechanical foaming agents, chemical foaming agents, foam stabilizers, and the like. Suitable dyes include, but are not limited to fiber reactive dyes or natural dyes. Suitable cross-linkers include, but are not limited to, epoxy-based cross-linkers, (for example, poly(ethylene glycol) diglycidyl ether (PEGDE) available from Sigma Aldridge), isocyanate-based cross-linkers (for example, X-TANR available from Lanxess), and carbodiimide-based cross-linkers. Suitable foaming agents include, HeiQ Chemtex 2216-T (a stabilized blend of nonionic and anionic surfactants), HeiQ Chemtex 2241-A (a modified HEUR (hydrophobically-modified ethylene oxide urethane) thickener), HeiQ Chemtex 2243 (a non-ionic silicone dispersion), or HeiQ Chemtex 2317 (a stabilized blend of nonionic and anionic surfactants) foam stabilizers available from HeiQ Chemtex. Suitable antimicrobial/antifungal agents include Ultra-Fresh DW-56, or other antimicrobial/antifungal agents used in the leather industry. Suitable flame retardants include CETAFLAMR DB9 (organophosphorous compounds containing C—PO(OH)₂ or C—PO(OR)₂ groups with the carbon chain containing polymers), CETAFLAMR PD3300 (organophosphorous compounds containing C—PO(OH)₂ or C—PO(OR)₂ groups with the carbon chain containing polymers), or other flame retardants used for coated textiles. Suitable fillers include, but are not limited to, thermoplastic microspheres, for example, EXPANCELR Microspheres. Suitable rheological modifiers include, but are not limited to, alkali swellable rheological modifiers, hydrophobically-modified ethylene oxide-based urethane (HEUR) rheological modifiers, and volume exclusion thickeners. Exemplary alkali swellable rheological modifiers include but are not limited to, ACRYSOL™ DR-106, ACRYSOL™ ASE-60 from Dow Chemicals, TEXICRYLR 13-3131, and TEXICRYL R 13-308 from Scott-Bader. Exemplary HEUR modifiers include, but are not limited to, RM-4410 from Stahl and Chemtex 2241-A from HeiQ. Exemplary volume exclusion thickeners include, but are not limited to, WALOCEL™ XM 20000 PV from Dow Chemicals and Methyl-Hydroxyethyl Cellulose from Sigma-Aldrich.

In some embodiments, a blend can include one or more coloring agents. In some embodiments, the coloring agent can be a dye, for example a fiber reactive dye, a direct dye, or a natural dye. Exemplary dyes, include but are not limited to, Azo structure acid dyes, metal complex structure acid dyes, anthraquinone structure acid dyes, and azo/diazo direct dyes. In some embodiments, the coloring agent can be pigment, for example a lake pigment. In some embodiments, a blend can include a coloring agent content of about 2 wt % or less. For example, a blend can include about 0.1 wt %, about 0.5 wt %, about 1 wt %, about 1.5 wt %, or about 2 wt % coloring agent. In some embodiments, a blend can include about 0.1 wt % to about 2 wt %, about 0.5 wt % to about 1.5 wt %, or about 0.1 wt % to about 1 wt % coloring agent. In some embodiments, a blend can be free or substantially free of a coloring agent. In such embodiments, a protein polyurethane alloy layer created from the blend can be free or substantially free of a coloring agent.

In step 2608, a layer of the blended mixture is disposed over top surface 2702 of sacrificial layer 2700. The blended mixture can be coated over top surface 2702 of sacrificial layer 2700. In embodiments not including steps 2602 and 2604, the blended mixture can be coated directly on top surface 2702 of sacrificial layer 2700. In embodiments including step 2604, the blended mixture can be coated directly on a surface of basecoat layer 2460. In embodiments including step 2602 but not step 2604, the blended mixture can be coated directly on a surface of top-coat layer 2470. In some embodiments, the blended mixture can be formed into a sheet by coating it on a surface to a desired thickness. Coating can include pouring, extruding, casting, and the like. In some embodiments, the sheet can be spread to a desired thickness using, for example, a blade, a knife, a roller, a knife over roll, curtain coating, and slot die coating.

In some embodiments, the temperature of the blended mixture during coating can be about 40° C. or higher. For example, the temperature of the blended mixture can range from about 40° C. to about 100° C., including subranges. For example, the temperature can be about 40° C., about 50° C., about 60° C., about 70° C., about 80° C., about 90° C., or about 100° C., or within a range having any two of these values as endpoints, inclusive of the endpoints. In some embodiments, the temperature of the blended mixture during coating can range from about 40° C. to about 90° C., about 40° C. to about 80° C., about 40° C. to about 70° C., about 40° C. to about 60° C., or about 40° C. to about 50° C. Coating at a temperature below about 40° C. can result in the blended mixture being too viscous and can make it difficult to form a layer of uniform thickness.

In step 2610, solvent (for example, water) can be removed from the coated blended mixture to form protein polyurethane alloy layer 2420, as illustrated in for example, FIG. 27C. Suitable solvent removal methods include, but are not limited to tunnel drying, vacuum drying, oven drying with hot air, humidity chamber drying, flotation drying with hot air, and ovens with a combination of medium range IR (infrared) for preheating and then hot air for subsequent drying.

Suitable solvent removal temperatures for step 2610 can range from about room temperature (18° C.) to about 100° C. Suitable humidity values for solvent removal in step 1910 include a humidity in a range from 0% RH (relative humidity) to about 65% RH. The solvent removal temperature and/or humidity can affect the final properties of a protein polyurethane alloy layer, and therefore a layered material. The solvent removal temperature and/or humidity in step 2610 can impact one or more of the following material properties: stiffness, elasticity, cohesive strength, tear strength, fire retardancy, chemical stability, and wet stability. For example, relatively high humidity and relatively low temperature can result in a material that is softer and more elastic. Conversely, relatively low humidity and relatively high temperature can result in a material that is harder and less elastic.

In some embodiments, steps 2606-2610 can be repeated a plurality of times to form a plurality of protein polyurethane alloy layers 2420 over sacrificial layer 2700. In some embodiments, steps 2606-2610 can be repeated sequentially to form a plurality of protein polyurethane alloy layers 2420 over sacrificial layer 2700. In some embodiments, steps 2606-2610 can be repeated after steps 2612-2616 to form one or more protein polyurethane alloy layers 2420 over one or more foamed protein polyurethane alloy layers 2430/2440. In some embodiments, method 2600 may not include steps 2606-2610.

In step 2612, one or more polyurethanes dispersed or dissolved in an aqueous solution can be blended with protein(s) and foamed to form a foamed blended mixture in the aqueous solution. In some embodiments, the one or more polyurethanes can be dispersed or dissolved in an aqueous solution before blending with protein(s) and foaming. In some embodiments, the one or more polyurethanes can become dispersed or dissolved in an aqueous solution during blending with protein(s) and foaming. In some embodiments, the one or more polyurethanes and the one or more proteins can be blended in a suitable vessel until a homogenous blend is formed. Suitable blending equipment includes, but is not limited to, a blender, a stand mixer, an in-line mixer, or a high shear mixer. The blend may be foamed using, for example, a mechanical foaming process or a chemical foaming process. Exemplary mechanical foaming equipment includes a Hansa Mixer or a GEMATA® foamer. Blending and foaming can be performed separately or concurrently.

Suitable polyurethane(s) for blending and foaming in step 2612 are those discussed herein for protein polyurethane alloy layers. In some embodiments, one or more foaming agents and/or foam stabilizers may be added to the blend in step 2612. Suitable foaming agents and foam stabilizers include those discussed herein for protein polyurethane alloy layers 2430/2440.

In some embodiments, a blend can include a foaming agent or a foam stabilizer content of about 10 wt % or less. For example, a blend can include about 0.1 wt %, about 1 wt %, about 2.5 wt %, about 5 wt %, about 7.5 wt %, or about 10 wt % foaming agent or foam stabilizer. In some embodiments, a blend can include about 0.1 wt % to about 10 wt %, about 1 wt % to about 7.5 wt %, about 2.5 wt % to about 5 wt %, about 0.1 wt % to about 5 wt %, or about 0.1 wt % to about 2.5 wt % foaming agent or foam stabilizer. In some embodiments, a blend can be substantially free or free of a foaming agent and/or a foam stabilizer. In such embodiments, a protein polyurethane alloy layer created from the blend can be substantially free or free of a foaming agent and/or a foam stabilizer.

Foaming in step 2612 can be used to impart a desired density for a foamed protein polyurethane alloy layer. In some embodiments, a foamed blended mixture can have a liquid density, before solvent is removed in step 2616, ranging from about 300 g/L to about 900 g/L, including subranges. For example, a foamed blended mixture formed in step 2612 can have a liquid density of about 300 g/L, about 400 g/L, about 500 g/L, about 600 g/L, about 700 g/L, about 800 g/L, or about 900 g/L, or within a range having any two of these values as endpoints. In some embodiments, the foamed blended mixture can have a liquid density ranging from about 300 g/L to about 800 g/L, about 300 g/L to about 700 g/L, about 400 g/L to about 600 g/L, about 300 g/L to about 500 g/L, or about 300 g/L to about 600 g/L. In some embodiments, a blended mixture formed in step 2606 can have a liquid density, before the solvent is removed from the blended mixture in step 2610 that is greater than the liquid density of the foamed blended mixture formed in step 2612 before solvent is removed in step 2616.

In some embodiments, protein(s) can be dispersed or dissolved in an aqueous solution before blending with polyurethane and foaming in step 2612. Suitable aqueous solutions include those discussed above for step 2606. The protein concentration in the aqueous solution can be any value or range discussed above for step 2606. The amount of protein in a protein/polyurethane blend for step 2612 can be any value or range discussed above for step 2606. The blending temperature for step 2612 can be in the range discussed above for step 2606. The blending time for step 2612 can be in the range discussed above for step 2606. The blending speed for step 2612 can be in the range discussed above for step 2606. In some embodiments, one or more additives can be added to the blend in step 2612. The additive(s) added in step 2612 can be any of the additives discussed above for step 2606.

In step 2614, a layer of the foamed blended mixture is disposed over sacrificial layer 2700. In some embodiments, a layer of the foamed blended mixture is disposed over a surface of a protein polyurethane alloy layer 2420. In some embodiments, the blended and foamed mixture can be coated directly on a surface of a protein polyurethane alloy layer 2420. In some embodiments, the foamed blended mixture can be formed into a sheet by coating it on a surface to a desired thickness. Coating can include pouring, extruding, casting, and the like. In some embodiments, the sheet can be spread to a desired thickness using, for example, a blade, a knife, a roller, a knife over roll, curtain coating, and slot die coating.

In step 2616, solvent (for example, water) can be removed from the coated, foamed blended mixture to form a foamed protein polyurethane alloy layer 2430, as illustrated in for example, FIG. 27D. Suitable solvent removal methods include, but are not limited to tunnel drying, vacuum drying, oven drying with hot air, humidity chamber drying, flotation drying with hot air, and ovens with a combination of medium range IR for preheating and then hot air for subsequent drying. Suitable solvent removal temperatures for step 2616 can in the temperature range discussed above for step 2610. Humidity values for step 2616 can be in the range discussed above for step 2610

In some embodiments, steps 2612-2616 can be repeated a plurality of times to form a plurality of foamed protein polyurethane alloy layers over sacrificial layer 2700, for example, foamed protein polyurethane alloy layers 2430 and 2440. In such embodiments, the foamed blended mixtures formed in separate steps 2612 can have different liquid densities. For example, the liquid density for one foamed blended mixture can be 10 g/L to 300 g/L more or less than the liquid density for another foamed blended mixture. For example, in some embodiments, a first blended mixture can have a liquid density ranging from about 300 g/L to about 500 g/L and a second blended mixture can have a liquid density ranging from about 600 g/L to about 700 g/L. As another example, a first blended mixture can have a liquid density ranging from about 300 g/L to about 400 g/L and a second blended mixture can have a liquid density ranging from about 500 g/L to about 700 g/L.

In some embodiments, steps 2612-2616 can be repeated sequentially to form a plurality of foamed protein polyurethane alloy layers over sacrificial layer 2700. In some embodiments, a foamed and blended mixture formed in step 2612 can be used to form multiple foamed protein polyurethane alloy layers in steps 2614-2616. In some embodiments, steps 2612-2616 can be performed before performing a set of steps 2606-2610 to form one or more foamed protein polyurethane alloy layers between a protein polyurethane alloy layer 2420 and sacrificial layer 2700. In some embodiments, method 1900 may not include steps 2612-2616.

In step 2618, sacrificial layer 2700 is removed from the layer(s) formed in steps 2602-2616, as illustrated in for example FIG. 27E. Sacrificial layer 2700 can be removed by a mechanical process or a chemical process. For example, sacrificial layer 2700 can be removed by peeling sacrificial layer 2700 away from the other layers. As another example, sacrificial layer 2700 can be removed by dissolving sacrificial layer 2700. In some embodiments, sacrificial layer 2700 can be removed in step 2618 before attaching the layer(s) formed in steps 2602-2616 to a substrate layer 2410 in step 2620. In some embodiments, sacrificial layer 2700 can be removed after step 2620.

In step 2620, the layer(s) formed in steps 2602-2616 are attached to a substrate layer 2410, as illustrated in for example FIG. 27F. In step 2620, protein polyurethane alloy layer 2420, and any other protein polyurethane alloy layers formed in steps 2606-2616 are attached to substrate layer 2410. In some embodiments, attaching one or more protein polyurethane alloy layers (e.g., protein polyurethane alloy layer 2420) to substrate layer 2410 in step 2620 includes a heat pressing process. In such embodiments, protein polyurethane alloy layer (e.g., protein polyurethane alloy layer 2420) can be in direct contact with substrate layer 2410. Also, in such embodiments, a protein polyurethane alloy layer can partially melt into substrate layer 2410, and upon cooling the two layers are firmly attached. In some embodiments, attaching one or more protein polyurethane alloy layers (e.g., protein polyurethane alloy layer 2420) to substrate layer 2410 in step 2620 includes a lamination process. In such embodiments, lamination can be accomplished with an adhesive layer 2450. In such embodiments, substrate layer 2410 and/or a protein polyurethane alloy layer can be coated with an adhesive by known techniques such as slot die casting, kiss coating, a drawdown technique, or reverse transfer coating. In some embodiments, the lamination process can include passing substrate layer 2410 and the other layer(s) through rollers under heat.

In some embodiments, step 2620 can be omitted from method 2600. In such embodiments, the layer(s) formed in steps 2602-2616 define a protein polyurethane alloy layer or a layered material without a substrate layer 2410.

In some embodiments, layered materials described herein can have a tear strength that is at least about 1% greater than that of a natural leather of the same thickness. For example, the layered material can have a tear strength that is about 1%, about 2%, about 3%, about 4%, about 5%, about 6%, about 7%, about 8%, about 9%, about 10%, about 15%, about 20%, about 25%, about 30%, about 35%, about 40%, about 45%, about 50%, about 100%, about 150%, or about 200% greater than that of natural leather of the same thickness. In some embodiments, the layered material can have a tear strength in the range of about 20 N to about 300 N, including subranges. For example, the tear strength of the layered material can be about 20 N, about 30 N, about 40 N, about 50 N, about 60 N, about 70 N, about 80 N, about 90 N, about 100 N, about 125 N, about 150 N, about 175 N, about 200 N, about 225 N, about 250 N, about 275 N, or about 300 N, or within a range having any two of these values as endpoints, inclusive of the endpoints. In some embodiments, the tear strength can range from about 30 N to about 275 N, about 40 N to about 250 N, about 50 N to about 225 N, about 60 N to about 200 N, or about 75 N to about 175 N, about 80 N to about 150 N, about 90 N to about 125 N, or about 100 N to about 125 N.

In some embodiments, a protein polyurethane alloy layer described herein can have a tear strength in the range of about 2 N to about 30 N, including subranges. For example, the tear strength of the protein polyurethane alloy layer can be about 2 N, about 4 N, about 5 N, about 10 N, about 15 N, about 20 N, about 25 N, or about 30 N, or within a range having any two of these values as endpoints, inclusive of the endpoints. In some embodiments, the tear strength can range from about 4 N to about 25 N, about 5 N to about 20 N, or about 10 N to about 15 N.

Tear strength, or tear resistance, is a measure of how well a material can withstand the effects of tearing. Tear resistance can be measured by a variety of methods, for example the method provided by ASTM D 412 or the method provided by ISO 3377 (also called the “Bauman tear”). The method provided by ASTM D 624 can also be used to measure the resistance to the formation of a tear and the resistance to the expansion of a tear. Regardless of the method used, first, a cut is made in the material sample tested to induce a tear. Then, the sample is held between two grips and a uniform pulling force is applied until sample tears in two. Tear resistance is then calculated by dividing the force applied by the thickness of the material. Unless specified otherwise, a tear strength value reported herein is measured by ISO 3377.

In some embodiments, the layered materials described herein can have a tensile strength in the range of about 1 kPa (kilopascal) to about 100 MPa (megapascals), including subranges. For example, the layered material can have a tensile strength of about 1 kPa, about 50 kPa, about 100 kPa, about 200 kPa, about 300 kPa, about 400 kPa, about 500 kPa, about 600 kPa, about 700 kPa, about 800 kPa, about 900 kPa, about 1 MPa, about 5 MPa, about 10 MPa, about 20 MPa, about 30 MPa, about 40 MPa, about 50 MPa, about 60 MPa, about 70 MPa, about 80 MPa, about 90 MPa, or about 100 MPa, or within a range having any two of these values as endpoints, inclusive of the endpoints. In some embodiments, the tensile strength can range from about 50 kPa to about 90 MPa, about 100 kPa to about 80 MPa, about 200 kPa to about 70 MPa, about 300 kPa to about 60 MPa, about 400 kPa to about 50 MPa, about 500 kPa to about 40 MPa, about 600 kPa to about 30 MPa, about 700 kPa to about 20 MPa, about 800 kPa to about 10 MPa, or about 1 MPa to about 5 MPa.

Softness, also referred to as “hand feel” of a material can be determined by ISO 17235. In some embodiments, an exterior surface of a layered material described herein can have a softness ranging from about 2 mm to about 12 mm, including subranges. For example, an exterior surface of a layered material can have a softness of about 2 mm, about 3 mm, about 4 mm, about 5 mm, about 6 mm, about 7 mm, about 8 mm, about 9 mm, about 10 mm, about 11 mm, or about 12 mm, or within a range having any two of these values as endpoints, inclusive of the endpoints. In some embodiments, the softness can be about 3 mm to about 11 mm, about 4 mm to about 10 mm, about 5 mm to about 9 mm, about 6 mm to about 8 mm, or about 6 mm to about 7 mm. Unless specified otherwise, a softness value disclosed herein is determined by ISO 17235.

Flexibility, or strain, of a material can be determined by measuring its elongation at failure when a tensile force is applied, for example using the equation: ΔL/L, where ΔL is the change in length of the material after the tensile force is applied, and L is the original length of the material. Flexibility can also be measured according to the method provided by ASTM D 412. In some embodiments, the layered materials described herein can have a flexibility in the range of about 100% to about 400%, including subranges. For example, the layered materials can have a flexibility of about 100%, about 200%, about 300%, or about 400%, or within a range having any two of these values as endpoints, inclusive of the endpoints. In some embodiments, the flexibility can be about 100% to about 200%, about 100% to about 300%, about 200% to about 300%, or about 200% to about 400%. Unless specified otherwise, a flexibility value disclosed herein is measured by ASTM D 412. In some embodiments, a protein polyurethane alloy layer described herein can have flexibility value or range as described above for a layered material.

In some embodiments, a layered material as described herein can have a permanent set in a hysteresis experiment of about 8% or less. In some embodiments, a layered material can have a permanent set of about 1%, about 2%, about 3%, about 4%, about 5%, about 6%, about 7%, or about 8%, or within a range having any two of these values as endpoints. In some embodiments, a layered material can have a permanent set of about 1% to about 8%, about 2% to about 7%, about 3% to about 6%, or about 4% to about 5%.

Unless specified otherwise, a permanent set value is measured by the following method. A dog-bone-shaped tensile specimen of a material is cut and the original length of the sample is measured. The samples are cut to have a dog-bone shape with about 110 mm length and 10 mm width (75-100 mm gauge length). Then, the sample is stretched along its length using an INSTRON® machine to 15% strain and returned to 0% strain, both at a constant rate of three millimeters per second. This is repeated five times. Then, the distance between the original sample length and the length of the sample at which the load goes to zero on the last return cycle is measured. The percent difference between the length measured after repeatedly straining the material and the original length is the permanent set %. For purposes of calculating a permanent set value, three separate samples of a material are evaluated, and the average permanent set value is reported as the permanent set value for the material.

In some embodiments, layered materials described herein can have a moisture vapor transmission rate (MVTR) of about 75 g/m²/hr or more. In some embodiments, layered materials described herein can have a MVTR ranging from about 75 g/m²/hr to about 200 g/m²/hr, including subranges. For example, the layered material can have a MVTR of about 80 g/m²/hr to about 190 g/m²/hr, about 90 g/m²/hr to about 180 g/m²/hr, about 100 g/m²/hr to about 170 g/m²/hr, about 110 g/m²/hr to about 160 g/m²/hr, about 120 g/m²/hr to about 150 g/m²/hr, or about 130 g/m²/hr to about 140 g/m²/hr. Unless specified otherwise, a MVTR value disclosed herein is measured using ASTM E96 (“Standard Test Methods for Water Vapor Transmission of Materials”)—Method B.

Layered materials having a moisture vapor transmission rate as reported herein can be suitable for use in a variety of applications where breathability of the material is a desirable property. Exemplary applications where breathability can be desirable include, but are not limited to, footwear, apparel, and upholstery. Layered materials as described herein can have a significantly higher water vapor transmission rate compared to a layered polymeric material having the same number of layers with the same thicknesses and made of the same polymeric material(s), but without protein blended in the polymeric material(s).

In some embodiments, layered materials described herein can have a color fastness of class 4 or higher when measured according to ISO 11640 (“Leather-Tests for color fastness-fastness to cycles of to-and-fro rubbing”) wet-rub fastness test. In some embodiments, layered materials described herein can have a color fastness of class 4, class 4.5, or class 5 when measured according to ISO 11640's wet-rub fastness test. A color fastness of class 4 or higher can provide layered materials described herein with desirable wear resistance for a variety of applications.

Layered materials described herein can achieve a color fastness of class 4 or higher without the inclusion of a pigment in the materials. This is a unique characteristic compared to a layered polyurethane material made of the same polyurethane(s) without protein(s) blended in the polyurethane(s). Protein within layered materials described herein can adhere well to a dye used to color the material. To achieve a high color fastness, polyurethane materials are usually colored using a pigment because dyes do not generally adhere to a polyurethane well. Poor adherence between a dye and a polyurethane leads to a relatively low color fastness. Dyed layered materials described herein can have improved depth of color and other aesthetic features not achievable with a polyurethane colored using a pigment.

In some embodiments, a layered material described herein, or an individual layer of a layered material described herein, can be subjected to the same, or similar finishing treatments as those used to treat natural leather. In some embodiments, a layered material described herein can be tumbled or staked to tailor properties of the material, such as the feel of the material. In such embodiments, traditional textile tumbling and staking methods can be used.

In some embodiments, a layered material, or an individual layer of a layered material, can have a rough exterior surface. For example, top surface 2424 of protein polyurethane alloy layer 2420 can have a rough surface, top surface 2474 of top-coat layer 2470 can have a rough surface, top surface 2464 of basecoat layer 2460 can have a rough surface, top surface 2434 of protein polyurethane alloy layer 2430 can have a rough surface, or top surface 2444 of protein polyurethane alloy layer 2440 can have a rough surface. A rough exterior surface can create a surface texture similar in appearance and feel to that of a naturel leather (e.g., the grain of pebbled natural leather). In some embodiments, top surface 2702 of sacrificial layer 2700 can have a rough surface that is transferred onto the surface of a layer disposed directly on top surface 2702 during method 2600.

A rough surface has a surface area per square inch of at least about 1% greater than 1 in². In other words, in some embodiments, a one square inch sample of layered material 2400, including a layer having rough exterior surface, can have a surface area that is at least about 1% greater than a one square inch sample of a material having a perfectly smooth surface. In some embodiments, a rough exterior surface can have a surface area per square inch of at least about 1% greater than 1 in², about 10% greater than 1 in², about 20% greater than 1 in², about 30% greater than 1 in², about 40% greater than 1 in², about 50% greater than 1 in², about 60% greater than 1 in², about 70% greater than 1 in², about 80% greater than 1 in², about 90% greater than 1 in², about 100% greater than 1 in², about 150% greater than 1 in², about 200% greater than 1 in², about 250% greater than 1 in², about 300% greater than 1 in², about 350% greater than 1 in², about 400% greater than 1 in², about 450% greater than 1 in², or about 500% greater than 1 in², or within a range having any two of these values as endpoints, inclusive of the endpoints. In some embodiments, a rough surface can have a surface area per square inch of about 1% greater than 1 in² to about 500% greater than 1 in², about 10% greater than 1 in² to about 450% greater than 1 in², about 20% greater than 1 in² to about 400% greater than 1 in², about 30% greater than 1 in² to about 350% greater than 1 in², about 40% greater than 1 in² to about 300% greater than 1 in², about 50% greater than 1 in² to about 250% greater than 1 in², about 60% greater than 1 in² to about 200% greater than 1 in², about 70% greater than 1 in² to about 150% greater than 1 in², or about 80% greater than 1 in² to about 100% greater than 1 in². Unless specified otherwise, a surface area of material disclosed herein is measured using profilometry. For non-transparent materials, optical profilometry is used. In some embodiments, a layered material, or an individual layer of a layered material, can have a smooth exterior surface. A smooth surface has a surface area per square inch of less than 1% greater than 1 in². For example, a smooth surface can have a surface area per square inch of 1 in² to less than 1.01 in². In some embodiments, top surface 2702 of sacrificial layer 2700 can have a smooth surface that is transferred onto the surface of a layer disposed directly on top surface 2702 during method 2600.

In some embodiments, a layered material, or an individual layer of a layered material, can have a textured exterior surface. In some embodiments, top surface 2702 of sacrificial layer 2700 can have a textured surface that is transferred onto the surface of a layer disposed directly on top surface 2702 during method 2600. In some embodiments, a textured exterior surface can a surface area per square inch, or surface area per square inch range, as discussed above for a rough surface.

In some embodiments, the texture can be a macro-scale texture, for example, any of the many textures used on Sappi/Warren Release Papers that are commercially available under the trademark ULTRACAST® or tradename Classic, manufactured by S.D. Warren Company d/b/a Sappi North America. An example of a macro-scale texture is a replicate of a natural leather grain with feature depths of about 50 to about 300 microns. Any other desired macro-scale texture may also be used. In some embodiments, a macro-scale texture can be a “leather grain texture.” As used herein, the term “leather grain texture” is a texture that mimics the look and feel of natural leather. Exemplary “leather grain textures” include but are not limited to, Sappi Matte Freeport 189, Sappi Freeport 123, or Sappi Expresso 904.

In some embodiments, the texture can be a micro-scale texture. In some embodiments, the texture can be a micro-scale texture with surface features having a feature size of less than 50 microns, for example 1000 nanometers to less than 50 microns. An example of a micro-scale texture is referred to in the art as “Sharklet.” Sharklet textures can be applied to provide the products with a surface that is structured to impede bacterial growth. The micro-scale texture of the surface replicates sharkskin denticles, which are arranged in a diamond pattern with millions of tiny ribs. Sharklet materials are discussed, for example, in U.S. Pat. Nos. 7,650,848 and 8,997,672, the disclosures of which are incorporated herein by reference.

In some embodiments, the texture can be a nanoscale texture with surface features having a feature size of less than 1000 nanometers, for example 10 nanometers to less than 1000 nanometers. One example of a nanoscale texture is a diffraction grating that has a series of raised ridges about 400 nanometers wide, spaced approximately 800 nanometers apart, with a depth of approximately 100 nanometers.

The embodiments discussed herein will be further clarified in the following examples. It should be understood that these examples are not limiting to the embodiments described above.

### Example 1

A 10% weight to volume (w/v) soy protein isolate (SPI) solution was prepared by adding 20 grams (g) of SPI (Solae XT 221D-IP; DuPont) in 180 milliliters (mL) of water to make a 200 mL solution. Then, 54 microliters (μL) of 10N sodium hydroxide (NaOH) was added and the solution was mixed until the SPI was fully dissolved. Once the SPI was fully dissolved, 200 milligrams (mg) of solid succinic anhydride and 10N sodium hydroxide (NaOH) were added simultaneously to the SPI solution while maintaining a pH of approximately 9 to 9.5. After addition of the succinic anhydride and sodium hydroxide, the solution was incubated at room temperature for 1 hour while being agitated using an overhead agitator. After incubation, the solution was dialyzed against DI water for 24 hours using SNAKESKIN™ dialysis tubing from Thermo Scientific.

After dialysis, the average lysine modification (%) and solubility (%) for the solution (Solution No. 1.1) was measured. Average lysine modification was determined using a lysine modification assay and solubility was determined by centrifugation.

For the solubility measurement, the modified protein was resuspended to a 5% (w/v) aqueous solution with DI water and the total solids were measured with a moisture analyzer. 35 mL of the 5% solution was centrifuged in a 50 mL tube for 10 minutes at 15,000×g (times gravity). After centrifugation, the supernatant was decanted and the volume and total solids of the supernatant (soluble fraction) was measured. The solubility was then calculated as follows: solubility=(total solids of the supernatant*volume of the supernatant)/(Total solids of the 5% starting solution*volume of the 5% starting solution). The measured solubility value for Solution No. 1.1 is shown below in Table 3.

For the lysine modification measurement, the protein samples were digested using trypsin before loading onto a liquid chromatography-mass spectrometer (LC/MS). After running the samples on the LC/MS, each sample was mapped by matching the digested peptides against the reference protein sequence for Beta-conglycinin alpha′ subunit (Gene CG-1; Organism: Glycine max (soybean) (Glycine hispida), which contains the possibility for both modified and unmodified lysine residues. Following peptide mapping, peak areas of matched peptides were used to calculate the proportion of modified to unmodified lysine at each lysine residue in the protein sequence and then the average % lysine modification was calculated based on all detected lysines. The following calculations were used to calculate the average % lysine modification. For each detected lysine, the lysine modification % was calculated as equal to the (SUM succinylated lysine/(SUM succinylated lysine+SUM non-succinylated lysine). The overall average % lysine modification was then calculated by averaging the individual lysine modification % of all the detected lysines. The measured average % lysine modification value for Solution 1.1 is shown below in Table 3.

The procedure for preparing Solution No. 1.1, described above, was repeated for Solution Nos. 1.2-1.12, with the amount of succinic anhydride shown in Table 3. The extent of lysine modification (%) and solubility (%) for each of Solution Nos. 1.2-1.12 was also measured using the same test methods as Solution No. 1.1. For succinic anhydride concentrations of 0.5% (Solution No. 1.5), 1.0% (Solution No. 1.10) and 1.5% (Solution No. 1.11), the lysine modification and solubility measurements were performed in quintuplicate (n=5), duplicate (n=2), and triplicate (n=3), respectively. The measured lysine modification and solubility values for Solution Nos. 1.1-1.12 are shown below in Table 3. The lysine modification and solubility values reported for Solution No. 1.5, Solution No. 1.10, and Solution No. 1.11 are the average values+/−their respective standard deviations.

TABLE 3

| | Succinic | Succinic | Average Lysine | |
|---|---|---|---|---|
| Solution | Anhydride | Anhydride | Modification | Solubility |
| No. | (w/v %) | Mass Added (g) | (%) | (%) |
| 1.1 | 0.1 | 0.2 | 23.3 | 51.5 |
| 1.2 | 0.2 | 0.4 | 33.1 | 56.1 |
| 1.3 | 0.3 | 0.6 | 41.4 | 57.1 |
| 1.4 | 0.4 | 0.8 | 63.8 | 68.1 |
| 1.5 | 0.5 (n = | 1.0 | 68.49 ± | 73.73 ± |
| | 5) | | 2.89 | 4.44 |
| 1.6 | 0.6 | 1.2 | 74.0 | 82.37 |
| 1.7 | 0.7 | 1.4 | 88.7 | 87.40 |
| 1.8 | 0.8 | 1.6 | 81.8 | 87.10 |
| 1.9 | 0.9 | 1.8 | 94.5 | 86.61 |
| 1.10 | 1 (n = | 2.0 | 95.38 ± | 86.85 ± |
| | 2) | | 0.68 | 1.2 |
| 1.11 | 1.5 (n = | 3.0 | 94.17 ± | 95.33 ± |
| | 3) | | 2.48 | 3.06 |
| 1.12 | 2 | 4.0 | 99.6 | 88.55 |

FIG. 1 shows a bivariate fit of solubility (%) versus succinic anhydride (w/v %) added to the protein solution for Sample Nos. 1.1-1.11 and FIG. 2 shows a bivariate fit of lysine succinylation (%) versus succinic anhydride (w/v %) for Sample Nos. 1.1-1.12. The results reported in Table 3 and FIGS. 1 and 2 demonstrate a positive linear relationship between 0.1% to 1% (w/v) succinic anhydride. At concentrations greater than 1% succinic anhydride, both the lysine modification and solubility plateau, suggesting limited or no additional lysine modification or solubility was achieved.

### Example 2

Three separate solutions (Solution Nos. 2.1-2.3) of 10% (w/v) soy protein isolate (SPI) were prepared by adding 20 g of SPI (Solae XT 221D-IP; DuPont) in 180 mL of water to make a 200 mL solution. Then 34 microliters (μL) of 10N sodium hydroxide (NaOH) was added to Solution No. 2.1, 48 μL of 10N NaOH was added to Solution No. 2.2, and 54 μL of 10N NaOH was added to Solution No. 2.3 to pH the solutions to about 7.5, about 8.5, and about 9.5, respectively, and all three solutions were mixed until the SPI was fully dissolved. Once the SPI was fully dissolved, 1 gram (0.5 w/v %) of solid succinic anhydride was added to each solution and each solution was mixed again. Then each solution was incubated at room temperature for 1 hour while being agitated using an overhead agitator. After incubation, each solution was dialyzed against DI water for 24 hours using SNAKESKIN™ dialysis tubing from Thermo Scientific.

After dialysis, the average lysine modification (%) and solubility (%) for each solution was measured as described above in Example 1. The measured average lysine modification (%) and mean solubility (%) values for each solution are shown below in Table 4. For the mean solubility values reported in Table 4, the solubility measurement was repeated three times on three separate 35 mL samples and the solubility is reported as the mean solubility of the three samples.

The results demonstrated a positive linear relationship between reaction pH and average lysine modification (%) as well as solubility (%). At a reaction pH of 9 to 9.5, the highest lysine modification and highest solubility were observed. Without being bound by theory, it is believed that the reaction may be slightly more efficient at a higher pH because the lysine is more likely to be deprotonated, which favors nucleophilic attack on succinic anhydride and results in succinyl lysine. Alternatively, and again without being bound by theory, the reaction may be slightly more efficient at a higher pH because the SPI is more soluble under alkaline conditions, thus making the lysine more accessible for modification.

TABLE 4

| | Succinylation | Average Lysine | Mean |
|---|---|---|---|
| Solution No. | Reaction pH | Modification (%) | Solubility (%) |
| 2.1 | 7-7.5 | 66.47 | 60 |
| 2.2 | 8-8.5 | 68.44 | 78 |
| 2.3 | 9-9.5 | 73.20 | 79 |

### Example 3A

A 10% (w/v) soy protein isolate (SPI) solution (Solution No. 3.1) was prepared by as described in Example 1. After incubation, the solution was dialyzed against DI water for 24 hours using SNAKESKIN™ dialysis tubing from Thermo Scientific and the excess sodium counter ion (referred to herein as “ash”) concentration was measured.

The procedure for preparing Solution No. 3.1 was repeated for Solution Nos. 3.2-3.9 with the respective amount of succinic anhydride varied as shown below in Table 4. Solution A was used as a negative control. Solution A was prepared by adding 20 g of SPI (Solae XT 221D-IP; DuPont) in 180 mL of water to make a 200 mL solution with the addition of 54 microliters (μL) of 10N sodium hydroxide. 10N sodium hydroxide was then added to the SPI solution to achieve a pH of approximately 9 to 9.5. After adding the sodium hydroxide, the solution was incubated at room temperature for 1 hour while being agitated using an overhead agitator. After incubation, the solution was dialyzed against DI water for 24 hours using SNAKEKIN™ dialysis tubing from Thermo Scientific and the excess sodium counter ion (ash) concentration was measured.

A theoretical calculation based on stoichiometry was used to predict the ash concentration and total protein yield at varying concentrations of succinic anhydride ranging from 0.0% to 2.0% (w/v) for non-dialyzed solutions. The results of the theoretical calculation for theoretical samples T1-T13 are shown below in Table 5. The calculations for the “theoretical sodium generated” in Table 5 are based on a 10% (w/v) SPI solution.

Table 6 shows the measured ash composition (g/100 g) for Solution Nos. A and 3.1-3.9 after dialysis. The ash composition (g/100 g) was measured using a standardized test called “ASHM_S.” In the ASHM_S test, organic matter is burned off by igniting a sample at 550° C. in an electric furnace. The mass of remaining material is determined gravimetrically and referred to as “ash.” The ASHM_S test results showed slightly lower ash concentrations than the theoretical predictions, which suggests that only a small amount of ash was removed during dialysis.

TABLE 5

| | | Succinic | | | | Theoretical | Theoretical |
|---|---|---|---|---|---|---|---|
| | | Anhydride | | Theoretical | Theoretical | Ash | Protein |
| Theoretical | Succinic | Mass | Succinic | Sodium | Sodium | Composition | Composition |
| Sample | Anhydride | Added | Anhydride | Hydroxide | Generated | (g/100 g) | (g/100 g) |
| Number | (w/v %) | (g) | (N) | (N) | (g/L)* | [Dry basis] | [Dry basis] |
| T1 | 0 | 0.0 | 0 | 0 | 0.00 | 4.99 | 90.71 |
| T2 | 0.1 | 0.2 | 0.02 | 0.02 | 0.46 | 5.43 | 90.30 |
| T3 | 0.2 | 0.4 | 0.04 | 0.04 | 0.92 | 5.86 | 89.89 |
| T4 | 0.3 | 0.6 | 0.06 | 0.06 | 1.38 | 6.28 | 89.48 |
| T5 | 0.4 | 0.8 | 0.08 | 0.08 | 1.84 | 6.71 | 89.08 |
| T6 | 0.5 | 1.0 | 0.1 | 0.1 | 2.30 | 7.12 | 88.68 |
| T7 | 0.6 | 1.2 | 0.12 | 0.12 | 2.76 | 7.54 | 88.28 |
| T8 | 0.7 | 1.4 | 0.14 | 0.14 | 3.22 | 7.95 | 87.89 |
| T9 | 0.8 | 1.6 | 0.16 | 0.16 | 3.68 | 8.36 | 87.50 |
| T10 | 0.9 | 1.8 | 0.18 | 0.18 | 4.14 | 8.76 | 87.12 |
| T11 | 1 | 2.0 | 0.2 | 0.2 | 4.60 | 9.16 | 86.73 |
| T12 | 1.5 | 3.0 | 0.3 | 0.3 | 6.90 | 11.11 | 84.87 |
| T13 | 2 | 4.0 | 0.4 | 0.4 | 9.20 | 12.98 | 83.09 |

TABLE 6

| | Succinic | | Theoretical | Measured |
|---|---|---|---|---|
| | Anhydride | | Ash | Ash |
| | Mass | Succinic | Composition | Composition |
| Solution | Added | Anhydride | (g/100 g) | (g/100 g) |
| No. | (g) | (w/v %) | [Dry basis] | [Dry basis] |
| A | 0.0 | 0 | 4.99 | 4.99 |
| 3.1 | 0.4 | 0.2 | 5.86 | 5.13 |
| 3.2 | 0.6 | 0.3 | 6.28 | 6.12 |
| 3.3 | 0.8 | 0.4 | 6.71 | 6.34 |
| 3.4 | 1.0 | 0.5 | 7.12 | 7.29 |
| 3.5 | 1.2 | 0.6 | 7.54 | 7.15 |
| 3.6 | 1.4 | 0.7 | 7.95 | 7.69 |
| 3.7 | 1.6 | 0.8 | 8.36 | 7.87 |
| 3.8 | 1.8 | 0.9 | 8.76 | 8.21 |
| 3.9 | 2.0 | 1 | 9.16 | 9.76 |

### Example 3B

Solution 3B.1, a 10% (w/v) soy protein isolate (SPI) solution, was prepared by adding 2 kg of SPI (Solae XT 221D-IP; DuPont) in 18 liters (L) of water to make a 20 L solution followed by 54 milliliters (mL) of 10N sodium hydroxide (NaOH) to enhance solubility. This solution was used directly for small molecule analysis as described below.

Solution 3B.2, a 10% (w/v) soy protein isolate (SPI) solution, was prepared by adding 2 kg of SPI (Solae XT 221D-IP; DuPont) in 18 liters (L) of water to make a 20 L solution followed by 54 milliliters (mL) of 10N sodium hydroxide (NaOH). Once the SPI was fully dissolved, the solution was succinylated by adding 100 grams (g) solid succinic anhydride (0.5 w/v %) and 10N sodium hydroxide (NaOH) simultaneously to the SPI solution while maintaining a pH of approximately 9 to 9.5. After addition of the succinic anhydride and sodium hydroxide, the solution was incubated at room temperature for 1 hour while being agitated using an overhead agitator. After incubation, solution 3B.2 was diluted to three times its volume with DI water. Next, the diluted solution was subject to two diavolume discontinuous diafiltrations followed by two diavolume continuous diafiltrations using a diafiltration instrument with a 50 kDa 46 mm (millimeter) spiral wound membrane spacer available from SNYDER® Filtration. Once the continuous diafiltrations were complete, the resulting diafiltered product was concentrated to 10% to 12% w/v solids in the diafiltration instrument. After concentration, the diafiltered product was spray dried into a solid protein powder.

Small molecule analysis was used to evaluate efficiency of the diafiltration process to remove phenolic color bodies from the protein solution. Solution No. 3B.2 Waste in Table 7 below represents the diafiltration waste collected from the succinylated protein solution, and Solution No. 3B.2 Product represents the final succinylated diafiltered protein product. The phenolic compound impurities in a sample of Solution 3B.1, Solution 3B.2 Waste, and Solution 3B.2 Product were measured using trimethylsilyl (TMS) derivatization for small molecules on a gas chromatography mass spectrometer. The phenolic compounds detected are summarized below in Table 7. As indicated by the measured phenolic compound impurities present in the diafiltration product, most of the phenolic compounds were removed by the diafiltration process. While these small molecules were not present in high quantities, they did introduce unwanted yellow color to the diafiltration waste.

TABLE 7

| Solution No. | Solution No. | Solution No. |
|---|---|---|
| 3B.1 (SPI) | 3B.2 Waste | 3B.2 Product |
| Syringic acid | Syringic acid | Pyrogallol |
| Flavones | Flavones | |
| Ferulic acid | Ferulic acid | |
| Emodin | Emodin | |
| Vanillic Acid | Vanillic Acid | |
| Maltol | Maltol | |
| 4-Cumylphenol/ | 4-Cumylphenol/ | |
| Bisphenol A (—OH) | Bisphenol A (—OH) | |
| Chrysophanol | 4-Hydroxybenzoic acid | |
| 1-Hydroxy-3-methoxy- | Hydroxyquinol | |
| 6-methylanthraquinone | | |
| 4-Hydroxyanthraquinone- | Sinapinic acid | |
| 2-carboxylic acid | | |
| | 4-Coumaric acid | |

### Example 4

Eight 10% (w/v) soy protein isolate (SPI) solutions (Solution Nos. 4.1-4.8) were prepared as described in Example 1 with the amounts of succinic anhydride shown below in Table 8. After incubation, each solution was dialyzed as described in Example 1. After dialysis, the product was lyophilized into a solid protein powder.

TABLE 8

| Solution | Succinic | Succinic Anhydride |
|---|---|---|
| No. | Anhydride (w/v %) | Mass Added (g) |
| 4.1 | 0 | 0 |
| 4.2 | 0.2 | 0.4 |
| 4.3 | 0.4 | 0.8 |
| 4.4 | 0.5 | 1.0 |
| 4.5 | 0.6 | 1.2 |
| 4.6 | 0.8 | 1.6 |
| 4.7 | 1.0 | 2.0 |
| 4.8 | 1.5 | 3.0 |

Eight protein polyurethane alloy solutions were prepared according to the following procedure, using the protein powder obtained from solutions 4.1-4.8 individually: 0.75 g of each solid protein powder described above was individually dissolved in 4.25 mL of deionized (DI) water and agitated with a stir bar. The solution was then vortexed to disperse the dissolved protein. After vortexing, the solution was heated to 50° C. and stirred at 600 rpm for 30 minutes. Then, 8.6 g (20:80 protein/polyurethane ratio) of waterborne polyurethane dispersion L3360 from Hauthaway was added to the solution and stirred without heat for an additional 20-30 minutes to remove bubbles and formed into a protein polyurethane film using a drawdown technique (described below).

A control polyurethane solution was prepared by mixing 8.6 g of a waterborne polyurethane dispersion L3360 from Hauthaway with 5 mL of de-ionized water. The solution was then warmed to 30-35° C. for 30 minutes and formed into a polyurethane film using the drawdown technique described below.

Protein polyurethane alloy drawdown films (Drawdown Sample Films 4.1-4.8) and a control polyurethane drawdown film (Drawdown Sample Film 4.9) were prepared using a TQC Automatic Film Applicator Model AB4120 with a 600 mm bar thickness, 15 mm/s speed, and a 250 mm length glass plate. The glass plates with the drawdown film were left in an oven at 75° C. to dry for 30 minutes. The films were then peeled off with a razor blade.

Each Drawdown Sample Film 4.1-4.9 was placed in a conditioning chamber (23° C., 50% humidity) overnight before testing. After conditioning, at least five dogbones of 10 mm width were cut from each of Drawdown Sample Films 4.1-4.8 and the tensile strength, Young's Modulus, and elongation at break (“elongation length”) of the samples were tested according to ISO3376 (“Leather—Physical and mechanical tests”), with the exception that the dogbone test samples had an overall width of 10 mm, a gauge length of 15 mm, and a gauge width of 5 mm. FIG. 3 is a graph of Young's Modulus for the Drawdown Sample Films 4.1-4.8. FIG. 4 is a graph of tensile strength for the Drawdown Sample Films 4.1-4.8. FIG. 5 is a graph of elongation length for the Drawdown Sample Films 4.1-4.8.

Samples cut from drawdown films 4.1, 4.4, 4.7, 4.8, and 4.9 were conditioned in an oven overnight at 45° C. After conditioning, a 10 mm wide strip was cut from each of the five samples and the strips were tested using a DMA-850 from TA Instruments across a temperature range of −80° C. to 200° C. and at a ramp rate of 4° C./min. FIG. 6 is a DMA thermogram comparing the DMA results for the five DMA samples.

At 0.6 w/v % succinic anhydride and above, the films became significantly more brittle, as evidenced by an increase in Young's modulus. Further, as evident in FIG. 6, increased succinic anhydride correlated to increased thermostability. Drawdown film 4.4 (0.5% w/v succinic anhydride) showed similar thermostability to drawdown film 4.1 (no succinic anhydride). At 1.0% w/v and 1.5% w/v succinic anhydride, the films exhibited a significantly higher thermostability in comparison to the film 4.1 (no succinic anhydride).

Drawdown films 4.1, 4.4, 4.7, and 4.8 exhibited significantly higher thermostability in comparison to Drawdown film 4.9. Drawdown film 4.1 had a second DMA modulus transition onset temperature of about 166.8° C. Drawdown film 4.4 had a second DMA modulus transition onset temperature of about 168.0° C. Drawdown film 4.7 had a second DMA modulus transition onset temperature of about 168.1° C. Drawdown film 4.8 had a second DMA modulus transition onset temperature of about 169.5° C. Drawdown film 4.9 had a second DMA modulus transition onset temperature of about 111.72° C. In addition, at 125° C., drawdown film 4.1 had a storage modulus about 35 MPa, drawdown film 4.4 had a storage modulus about 52 MPa, drawdown film 4.7 had a storage modulus about 58 MPa, drawdown film 4.8 had a storage modulus about 154 MPa, and drawdown film 4.9 had a storage modulus about 0.9 MPa.

### Example 5

Eight 10% (w/v) soy protein isolate (SPI) solutions (Solution Nos. 5.1-5.8) were prepared as described in Example 1 with the amounts of succinic anhydride shown below in Table 9. After incubation, each solution was dialyzed as described in Example 1. After dialysis, the product was lyophilized into solid protein powder.

TABLE 9

| Solution | Succinic | Succinic Anhydride |
|---|---|---|
| No. | Anhydride (w/v %) | Mass Added (g) |
| 5.1 | 0 | 0 |
| 5.2 | 0.1 | 0.2 |
| 5.3 | 0.3 | 0.6 |
| 5.4 | 0.5 | 1.0 |
| 5.5 | 0.6 | 1.2 |
| 5.6 | 0.8 | 1.4 |
| 5.7 | 1.0 | 2.0 |
| 5.8 | 1.5 | 3.0 |

Each respective protein powder obtained from Solution Nos. 5.1-5.8 was then dissolved in deionized water at a concentration of 150 mg/mL to create eight different sample protein solutions. The complex viscosity, and loss and storage moduli for each of the eight protein solutions were measured by a TA DHR-2 rheometer from TA Instruments with a concentric cylinder. For each measurement, 10 mL of each protein solution was loaded into the cylinder and a frequency sweep test was performed at the range of 0.1 to 100 rad/s to read the complex viscosity, and loss and storage moduli values.

FIG. 7 is a graph of the complex viscosity for the eight protein solutions. The succinylated protein solutions' viscosities increased as the amount of added succinic anhydride increased. Protein solutions with 0.1 to 0.3 w/v % added succinic anhydride showed similar protein solubility in water compared to the non-succinylated protein solution (Solution No. 5.1). Protein solutions with 0.5 to 1.5 w/v % added succinic anhydride powder exhibited increased viscosity compared to protein solutions with 0.1 to 0.3 w/v % succinic anhydride.

FIG. 8 is a graph of the loss and storage moduli for the sample protein solutions made with the solid protein particles obtained from Solution Nos. 5.2, 5.3, 5.4, 5.7, and 5.8. At 0.1 w/v % and 0.3 w/v % added succinic anhydride, the protein solutions had a viscous behavior. At 0.5 w/v % added succinic anhydride and above, a more elastic behavior was observed. The graphs in FIGS. 7 and 8 suggest protein solubility may be saturated at about 0.5 w/v % added succinic anhydride.

### Example 6

Ten 10% (w/v) soy protein isolate (SPI) solutions (Solution Nos. 6.1-6.1.0) were prepared as described in Example 1 with the amounts of succinic anhydride shown below in Table 10. After incubation, each solution was dialyzed as described in Example 1. After dialysis, the product was lyophilized into solid protein powder.

TABLE 10

| Solution | Succinic | Succinic Anhydride |
|---|---|---|
| No. | Anhydride (w/v %) | Mass Added (g) |
| 6.1 | 0 | 0 |
| 6.2 | 0.1 | 0.2 |
| 6.3 | 0.2 | 0.4 |
| 6.4 | 0.3 | 0.6 |
| 6.5 | 0.4 | 0.8 |
| 6.6 | 0.5 | 1.0 |
| 6.7 | 0.7 | 1.4 |
| 6.8 | 0.9 | 1.8 |
| 6.9 | 1.0 | 2.0 |
| 6.10 | 1.5 | 3.0 |

Ten alloy solutions were prepared using Solution Nos. 6.1-6.10, respectively, as described in Example 4. After the protein polyurethane solutions were prepared, their complex viscosity and their loss and storage moduli were measured as described above in Example 5.

FIGS. 9A and 9B are graphs of the complex viscosity for the ten protein polyurethane alloy solutions. As shown in FIGS. 9A and 9B, protein polyurethane alloy solutions made with 0.1 to 0.5% w/v added succinic anhydride displayed a Newtonian behavior. In contrast, protein polyurethane alloy solutions made with 0.5%-1.5% w/v added succinic anhydride powder displayed a shear thinning behavior.

FIG. 10 is a graph of the loss and storage moduli for the protein polyurethane alloy solutions made with the spray dried protein particles obtained from Solution Nos. 6.1, 6.2, 6.6, 6.9, and 6.10. The protein polyurethane alloy solutions' storage modulus (G′) and loss modulus (G″) increased as the amount of added succinic anhydride increased. Also, while the solution made with 1.5% w/v added succinic anhydride powder had similar viscosity to the solution made with no added succinic anhydride powder (see FIGS. 9A and 9B), its storage modulus (G′) was higher than its loss modulus (G″). Without wishing to be bound by theory, the observed behavior for complex viscosity, loss modulus, and storage modulus suggests there may be increased network forming between the polyurethane and the protein at higher degrees of succinylation for the protein powder.

### Example 7

Eight 10% (w/v) soy protein isolate (SPI) solutions (Solution Nos. 7.1-7.8) were prepared as described in Example 1 with the amounts of succinic anhydride shown below in Table 11. After incubation, each solution was dialyzed as described in Example 1. After dialysis, the product was lyophilized into solid protein powder.

TABLE 11

| Solution | Succinic | Succinic Anhydride |
|---|---|---|
| No. | Anhydride (w/v %) | Mass Added (g) |
| 7.1 | 0 | 0 |
| 7.2 | 0.2 | 0.4 |
| 7.3 | 0.4 | 0.8 |
| 7.4 | 0.5 | 1.0 |
| 7.5 | 0.6 | 1.2 |
| 7.6 | 0.8 | 1.6 |
| 7.7 | 1.0 | 2.0 |
| 7.8 | 1.5 | 3.0 |

Eight protein polyurethane alloy solutions were prepared as described in Example 4 using Solution Nos. 7.1-7.8, respectively. Then the eight protein polyurethane alloy solutions were formed into protein polyurethane films using the drawdown technique as described in Example 4.

The eight drawdown films were then prepared for color testing by taping down the top and bottom edges of the films with paper tape. Each film's initial color was measured with a Hunter Lab UltraScan spectrophotometer set at mode: RSIN-Reflectance Specular Included (% R UVN SCI). Then each film was placed in a hydrolysis chamber at 70° C. and 95% humidity for six days. After removal from the hydrolysis chamber, each film was placed in an oven at 45° C. for 10 min to dry. The color of each film was then measured with the UltraScan spectrophotometer (% R UVN SCI mode), using a white tile as a standard. The color parameter “dE*” measured by the spectrophotometer was recorded. The color of a sample measured with the UltraScan spectrophotometer consists of three spectral components-light/dark (L*), red/green (a*), and blue/yellow (b*). “dE*” is the overall color change with the equation:

$${dE}^{*} = \sqrt{{( {L}_{2}^{*} - {L}_{1}^{*} )}^{2} + {( {a}_{2}^{*} - {a}_{1}^{*} )}^{2} + {( {b}_{2}^{*} - {b}_{1}^{*} )}^{2}} ,$$

where 2 is the sample film and 1 is the white tile standard.

The graph in FIG. 11 shows the difference in color (ΔE, dE*) for each of the films on the sixth day. The difference in color values plotted are the difference in color relative to the white tile used as the standard on the sixth day. The difference in color was measured at least twice on the sixth day for each film. The results showed a strong correlation between the amount of added succinic anhydride and an anti-yellowing effect. As the amount of added succinic anhydride was increased, the films exhibited a lower initial color difference and a lower final color difference.

### Example 8

Eight 10% (w/v) soy protein isolate (SPI) solutions (Solution Nos. 8.1-8.8) were prepared as described in Example 1 with the amounts of succinic anhydride and at the pH values shown below in Table 12. After incubation, each solution was dialyzed as described in Example 1. After dialysis, the product was lyophilized into a solid protein powder.

TABLE 12

| | Succinic | Succinic | |
|---|---|---|---|
| Solution | anhydride | anhydride | pH of |
| No. | (w/v %) | added (g) | solution |
| 8.1 | 0 | 0 | 9.5 |
| 8.2 | 0.5 | 1.0 | 9.5 |
| 8.3 | 0.5 | 1.0 | 7.5 |
| 8.4 | 0.5 | 1.0 | 8.5 |
| 8.5 | 0.5 | 1.0 | 9.5 |
| 8.6 | 1.0 | 2.0 | 7.5 |
| 8.7 | 1.0 | 2.0 | 8.5 |
| 8.8 | 1.0 | 2.0 | 9.5 |

Eight protein polyurethane alloy solutions were prepared as described in Example 4 using Solution Nos. 8.1-8.8, respectively. Then the eight protein polyurethane alloy solutions were formed into protein polyurethane films using the drawdown technique described in Example 4.

Each drawdown film was then placed in a conditioning chamber (23° C., 50% humidity) overnight before testing. After conditioning, at least five dogbones of each film were cut and the tensile strength, Young's Modulus, and elongation at break (“elongation length”) of the samples was tested according to ISO3376 (“Leather—Physical and mechanical tests”), with the exception that the dogbone test samples had an overall width of 10 mm, a gauge length of 15 mm, and a gauge width of 5 mm FIG. 12 is a graph of Young's Modulus for each of the eight films. FIG. 13 is a graph of tensile strength for each of the eight films. FIG. 14 is a graph of elongation length for each of the eight films.

At 1.0 w/v % succinic anhydride, the Young's Modulus varied between about 125 MPa and about 160 MPa at the different reaction pHs. At 0.5 w/v % succinic anhydride, the Young's Modulus varied between about 100 MPa and about 125 MPa. Similar to the results for tensile strength, there was less variation in Young's Modulus for 0.5 w/v % succinic anhydride compared to 1.0 w/v % succinic anhydride. In general, the films became stiffer with increasing reaction pH.

At 1.0 w/v % succinic anhydride, the tensile strength varied between about 9 MPa and about 17 MPa at the different reaction pHs. At 0.5 w/v % succinic anhydride, the tensile strength varied between about 12 MPa and 18 MPa at the difference reaction pHs. Overall, there was less variation in the tensile strength for 0.5 w/v % succinic anhydride compared to 1.0 w/v % succinic anhydride.

At 1.0 w/v % succinic anhydride, the elongation length varied between about 50% and 275% at the difference reaction pHs. At 0.5 w/v % succinic anhydride, the elongation length varied between about 125% and about 275%. Similar to the results for tensile strength and Young's Modulus, there was less variation in elongation length for 0.5 w/v % succinic anhydride compared to 1.0 w/v % succinic anhydride.

### Example 9

Seven 10% (w/v) soy protein isolate (SPI) solutions (Solution Nos. 9.1-9.7) were prepared as described in Example 1 with the amounts of succinic anhydride and at the pH values shown below in Table 13. After incubation, each solution was dialyzed as described in Example 1. After dialysis, the product was lyophilized into a solid protein powder.

TABLE 13

| Solution | Succinic | Succinic anhydride | |
|---|---|---|---|
| No. | anhydride (w/v %) | added (g) | pH |
| 9.1 | 0.5 | 1.0 | 9.5 |
| 9.2 | 0.5 | 1.0 | 7.5 |
| 9.3 | 0.5 | 1.0 | 8.5 |
| 9.4 | 0.5 | 1.0 | 9.5 |
| 9.5 | 1.0 | 2.0 | 7.5 |
| 9.6 | 1.0 | 2.0 | 8.5 |
| 9.7 | 1.0 | 2.0 | 9.5 |

Seven protein polyurethane alloy solutions were prepared as described in Example 4 using Solution Nos. 9.1-9.7, respectively.

The complex viscosity of the seven protein polyurethane alloy solutions was measured as described above in Example 5.

FIG. 15 is a graph of the complex viscosity for the seven protein polyurethane alloy solutions. The complex viscosity measurements show a noticeable increase in viscosity at 1.0 w/v % succinic anhydride, with the viscosity increasing with increasing pH. Without wishing to be bound by theory, it is possible the increase in viscosity could be an indicator of a destabilized polyurethane dispersion.

### Example 10

Eight 10% (w/v) soy protein isolate (SPI) solutions (Solution Nos. 10.1-10.8) were prepared as described in Example 1 with the amounts of succinic anhydride and at the pH values shown below in Table 14. After incubation, each solution was dialyzed as described in Example 1. After dialysis, the product was lyophilized into a solid protein powder.

TABLE 14

| Solution | Succinic anhydride | Succinic anhydride | |
|---|---|---|---|
| No. | (w/v %) | added (g) | pH of solution |
| 10.1 | 0 | 0 | 9.5 |
| 10.2 | 0.5 | 1.0 | 9.5 |
| 10.3 | 0.5 | 1.0 | 7.5 |
| 10.4 | 0.5 | 1.0 | 8.5 |
| 10.5 | 0.5 | 1.0 | 9.5 |
| 10.6 | 1.0 | 2.0 | 7.5 |
| 10.7 | 1.0 | 2.0 | 8.5 |
| 10.8 | 1.0 | 2.0 | 9.5 |

Eight separate protein polyurethane alloy solutions were prepared as described in Example 4 using Solution Nos. 10.1-10.8, respectively. Then the eight protein polyurethane alloy solutions were formed into a protein polyurethane film using the drawdown technique as described in Example 4.

The drawdown films were then prepared for color testing by taping down the top and bottom edges with paper tape. Each film's initial color was measured with a Hunter Lab UltraScan spectrophotometer set at mode: RSIN-Reflectance Specular Included (% R UVN SCI). Then each film was placed in a hydrolysis chamber at 70° C. and 95% humidity. The next day, each sample was removed from the hydrolysis chamber and placed in an oven at 45° C. for 10 min to dry. The color of each film was then measured with the UltraScan % R UVN SCI mode, using a white tile as a standard. After measuring their color, each film was again placed in the hydrolysis chamber at 70° C. and 95% humidity. This color was tracked over seven days.

FIG. 16 shows the blueness-yellowness difference (Ab*, db*) defined by the spectrometer measured b* value in CIELab color space between the eight films and the white tile on the seventh day (i.e., after six days in the hydrolysis chamber). All seven films made with succinylated SPI exhibited less change in their b* value compared to the film made with non-succinylated SPI. The results also show that reaction pH can have an influence on color change performance.

### Example 11

Two non-succinylated soy protein isolate (SPI) polyurethane solutions were prepared as described in Example 4 using Solae XT 221D-IP as the protein powder and L3360 from Hauthaway as the polyurethane with the addition of 25 μL of 10N NaOH to assist with dissolution of the protein in the solutions. Then, two succinylated soy protein isolate (S-SPI) polyurethane solutions were prepared as described in Example 4 using the spray-dried protein powder obtained from Example 3B and L3360 from Hauthaway without the addition of 25 μL of 10N NaOH.

After addition of the polyurethane, 1% (based on weight of protein) of Turquoise fiber reactive dye was added to one of the non-succinylated protein polyurethane solutions and one of the succinylated protein polyurethane solutions. Separately, 1% (based on weight of protein) of Bordeaux fiber reactive dye was added to one of the non-unsuccinylated (SPI) protein polyurethane solutions and one of the succinylated (S-SPI) protein polyurethane solutions. The dyes used were Bright Turquoise CW #20, 77.2 μL of 97 mg/mL solution in water (“Turquoise”) and Bordeaux CW #7479, 82.4 μL of 91 mg/mL solution in water (“Bordeaux”). After the dye was added, the solutions were stirred without heat at no higher than 150 rpm for another 20 to 30 minutes to remove bubbles. Then the solutions were made into protein polyurethane alloy drawdown films according to the drawdown technique as described in Example 4.

The protein polyurethane alloy drawdown films were prepared for aging testing by taping the top and bottom edges with paper tape. Each film's initial color (Day 0) was measured with a Hunter Lab UltraScan spectrophotometer set at mode: RSIN-Reflectance Specular Included (% R UVN SCI). The samples were then left in a hydrolysis chamber (70° C., 95% humidity). Before each color measurement, the film was removed from the chamber and left in an oven at 45° C. for 10 minutes to dry. The sample's color was tracked over 14 days.

The measured color values for the Turquoise and Bordeaux films over 14 days are shown in FIGS. 17A and 17B, respectively. Negative b* values represent the degree of blueness and positive b* values represent a degree of yellowness defined by the spectrometer measured b* value in CIELab color space. Positive a* values represent the degree of redness and negative a* values represent a degree of greenness defined by the spectrometer measured a* value in CIELab color space.

For both dyes, the succinylated SPI films (“S-SPI Turquoise” and “S-SPI Bordeaux”) displayed more of their desired color at Day 0 and retained their dye's color better over 14 days in comparison to non-succinylated SPI films (“SPI Turquoise” and “SPI Bordeaux”). The more negative b* values for the succinylated SPI in FIG. 17A indicate the film was better at displaying the Turquoise dye color and retaining the color over time. The higher a* values for the succinylated SPI in FIG. 17B indicate the film was better at displaying the Bordeaux dye color and retaining the color over time.

### Example 12

Two protein polyurethane alloy materials were prepared. The first material was prepared by adding 8.8 g of Solae XT 221D-IP and 250 μL (microliter) of 10N NaOH solution into 50 mL of DI water. The solution was then heated to 50° C. for 30 min to fully dissolve the protein powder. Once dissolved, 1.2 g of antimicrobial Ultra-Fresh DW-56 was added and the solution was stirred for five minutes at room temperature. After stirring, 53 g of waterborne polyurethane dispersion IMPRAPERMR DL 5249 from Covestro was added to the solution, resulting in 30:70 protein:polyurethane ratio. After addition of the polyurethane, 0.5 g of AF-715 (an antifoaming agent available from Quaker Color) was added and the solution was stirred at room temperature for 5 minutes until fully mixed. Once mixed, 0.2 g of BORCHI® Gel L 75 N was added and the solution was again stirred at room temperature for 15 minutes. The solution was then coated on release paper with a gap width of 0.38 mm using a Mathis LABCOATER and dried at 75° C. for 10 minutes. After drying, another layer of the same solution was added with a gap width of 0.45 mm and dried for 10 minutes at 75° C. followed by 10 minutes at 100° C. The dried material was then removed from the release paper. After drying, the material had a thickness ranging from 0.08 mm and 0.1 mm.

The second material was prepared by adding 8.8 g of the spray-dried protein powered obtained from Example 3B into 50 mL of DI water. The solution was then heated to 50° C. for 30 min to fully dissolve the protein powder. Once dissolved, 1.2 g of antimicrobial Ultra-Fresh DW-56 was added and the solution was stirred for five minutes at room temperature. After stirring, 53 g of waterborne polyurethane dispersion IMPRAPERMR DL 5249 from Covestro was added to the solution, resulting in 30:70 protein:polyurethane ratio. After addition of the polyurethane, 0.5 g of AF-715 (an antifoaming agent available from Quaker Color) was added and the solution was stirred at room temperature for 5 minutes until fully mixed. Once mixed, 0.2 g of Borchi® Gel L 75 N was added and the solution was again stirred at room temperature for 15 minutes. The solution was then coated on release paper with a gap width of 0.38 mm using a Mathis LABCOATER and dried at 75° C. for 10 minutes. After drying, another layer of the same solution was added with a gap width of 0.45 mm and dried for 10 minutes at 75° C. followed by 10 minutes at 100° C. The dried material was then removed from the release paper. After drying, the material had a thickness ranging from 0.08 mm and 0.1 mm.

Three samples (Samples 12.1.1, 12.2.1 and 12.2.2) were then measured for breathability according to ASTM E96-Method B. Sample 12.1.1 was a 0.08 mm thick piece of the first material. Sample 12.2.1 was a 0.1 mm thick piece of the second material. Sample 12.2.2 was a 0.08 mm thick piece of the second material.

FIG. 18 shows the average moisture vapor transmission rate (MVTR) measured in g/m²/24 hr for each of the three samples. Samples 12.2.1 and 12.2.2 exhibited improved breathability compared to Sample 12.1.1. Sample 12.1.1 had an average MVRT of 520 g/m²/24 h, with a maximum measured value of 564 g/m²/24 h. Sample 12.2.1 and Sample 12.2.2 had an average MVTR of 561 g/m²/24 h and 584 g/m²/24 h, respectively. Sample 12.2.2 had a maximum measured value of 603 g/m²/24 h.

### Example 13

Two protein polyurethane alloy solutions were prepared as described in Example 4 using non-succinylated Solae XT 221D-IP and the spray-dried protein powder obtained from Example 3B, respectively, and water borne polyurethane dispersion RC-2214 from Stahl as the polyurethane. Then the two protein polyurethane alloy solutions were formed into protein polyurethane films (Drawdown Sample Films 13.1 and 13.2) using the drawdown technique as described in Example 4.

A third sample protein polyurethane alloy mixture was prepared as described in Example 4 using the spray-dried protein powder obtained from Example 3B and water borne polyurethane dispersion 414 from Stahl as the polyurethane. This third mixture exhibited phase separation and immiscibility, and did not form a protein polyurethane alloy solution. As such, the third mixture was not formed into a protein polyurethane film.

A control polyurethane solution, was prepared by mixing 5.1 g of a waterborne polyurethane dispersion RC-2214 from Stahl with 5 mL of de-ionized water. The solution was then warmed to 30-35° C. for 30 minutes and formed into a polyurethane film (Drawdown Sample Film 13.3) using the drawdown technique described in Example 4.

Drawdown Sample Film 13.2 and 13.3 were placed in a conditioning chamber (23° C., 50% humidity) overnight before testing. After conditioning, at least five dogbones were cut from each of Drawdown Film Sample Nos. 13.1 and Sample No. 13.2, respectively, and the tensile strength, Young's Modulus, and elongation at break (“elongation length”) of the films was tested according to according to ISO3376 (“Leather—Physical and mechanical tests”), with the exception that the dogbone test samples had an overall width of 10 mm, a gauge length of 15 mm, and a gauge width of 5 mm. FIG. 19 is a graph of Young's Modulus for Drawdown Film Sample Nos. 13.1 and Sample No. 13.2. FIG. 20 is a graph of tensile strength for Drawdown Film Sample Nos. 13.1 and Sample No. 13.2. FIG. 21 is a graph of elongation length for Drawdown Film Sample Nos. 13.1 and Sample No. 13.2.

Samples cut from Drawdown Films Sample Nos. 13.1, 13.2, and 13.3 were conditioned oven overnight at 45° C. After conditioning, a 10 mm wide strip was cut from each of the three samples and the strips were tested using a DMA-850 from TA Instruments across a temperature range of −80° C. to 200° C. and at a ramp rate of 4° C./min. FIG. 22 is a DMA thermogram comparing the DMA results for the three DMA samples.

The data in FIGS. 19-21 shows that the film made with succinylated SPI was stiffer and weaker compared to the film made with non-succinylated SPI. The thermogram in FIG. 22 shows that the film made with succinylated SPI and the film made with non-succinylated SPI had similar thermostability. Although the film made with succinylated SPI and the film made with non-succinylated SPI did not show a significant increase in second DMA modulus transition onset temperature compared to the control sample, the widening of the rubbery plateau in the thermogram and an increase in Young's modulus indicate the SPI was dissolved in the RC-2214 polyurethane.

While various embodiments have been described herein, they have been presented by way of example, and not limitation. It should be apparent that adaptations and modifications are intended to be within the meaning and range of equivalents of the disclosed embodiments, based on the teaching and guidance presented herein. It therefore will be apparent to one skilled in the art that various changes in form and detail can be made to the embodiments disclosed herein without departing from the spirit and scope of the present disclosure. The elements of the embodiments presented herein are not necessarily mutually exclusive, but can be interchanged to meet various situations as would be appreciated by one of skill in the art.

Embodiments of the present disclosure are described in detail herein with reference to embodiments thereof as illustrated in the accompanying drawings, in which like reference numerals are used to indicate identical or functionally similar elements. References to “one embodiment,” “an embodiment,” “some embodiments,” “in certain embodiments,” etc., indicate that the embodiment described can include a particular feature, structure, or characteristic, but every embodiment can not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

The examples are illustrative, but not limiting, of the present disclosure. Other suitable modifications and adaptations of the variety of conditions and parameters normally encountered in the field, and which would be apparent to those skilled in the art, are within the spirit and scope of the disclosure.

It is to be understood that the phraseology or terminology used herein is for the purpose of description and not of limitation. The breadth and scope of the present disclosure should not be limited by any of the above-described exemplary embodiments, but should be defined in accordance with the following claims and their equivalents.

### SEQUENCES

Human Collagen alpha-1(III) chain
SEQ ID NO: 1:
DVKSGVAVGGLAGYPGPAGPPGPPGPPGTSGHPGSPGSPGYQGPPGEPGQ
AGPSGPPGPPGAIGPSGPAGKDGESGRPGRPGERGLPGPPGIKGPAGIPG
FPGMKGHRGFDGRNGEKGETGAPGLKGENGLPGENGAPGPMGPRGAPGER
GRPGLPGAAGARGNDGARGSDGQPGPPGPPGTAGFPGSPGAKGEVGPAGS
PGSNGAPGQRGEPGPQGHAGAQGPPGPPGINGSPGGKGEMGPAGIPGAPG
LMGARGPPGPAGANGAPGLRGGAGEPGKNGAKGEPGPRGERGEAGIPGVP
GAKGEDGKDGSPGEPGANGLPGAAGERGAPGFRGPAGPNGIPGEKGPAGE
RGAPGPAGPRGAAGEPGRDGVPGGPGMRGMPGSPGGPGSDGKPGPPGSQG
ESGRPGPPGPSGPRGQPGVMGFPGPKGNDGAPGKNGERGGPGGPGPQGPP
GKNGETGPQGPPGPTGPGGDKGDTGPPGPQGLQGLPGTGGPPGENGKPGE
PGPKGDAGAPGAPGGKGDAGAPGERGPP

Collagen Fragment
SEQ ID NO: 2:
DVKSGVAVGGLAGYPGPAGPPGPPGPPGTSGHPGSPGSPGYQGPPGEPGQ
AGPSGPPGPPGAIGPSGPAGKDGESGRPGRPGERGLPGPPGIKGPAGIPG
FPGMKGHRGFDGRNGEKGETGAPGLKGENGLPGENGAPGPMGPRGAPGER
GRPGLPGAAGARGNDGARGSDGQPGPPGPPGTAGFPGSPGAKGEVGPAGS
PGSNGAPGQRGEPGPQGHAGAQGPPGPPGINGSPGGKGEMGPAGIPGAPG
LMGARGPPGPAGANGAPGLRGGAGEPGKNGAKGEPGPRGERGEAGIPGVP
GAKGEDGKDGSPGEPGANGLPGAAGERGAPGFRGPAGPNGIPGEKGPAGE
RGAPGPAGPRGAAGEPGRDGVPGGPGMRGMPGSPGGPGSDGKPGPPGSQG
ESGRPGPPGPSGPRGQPGVMGFPGPKGNDGAPGKNGERGGPGGPGPQGPP
GKNGETGPQGPPGPTGPGGDKGDTGPPGPQGLQGLPGTGGPPGENGKPGE
PGPKGDAGAPGAPGGKGDAGAPGERGPPAIAGIGGEKAGGFAPYYG

What is claimed is:

1. A protein polyurethane alloy, comprising:
a succinylated protein dissolved within a polyurethane.

2. The protein polyurethane alloy of claim 1, wherein the succinylated protein is a succinylated soy protein.

3. The protein polyurethane alloy of claim 2, wherein the succinylated soy protein is succinylated soy protein isolate.

4. The protein polyurethane alloy of claim 2 or claim 3, wherein the protein polyurethane alloy has a second DMA modulus transition onset temperature ranging from about 130° C. to about 200° C.

5. The protein polyurethane alloy of any one of claims 2-4, wherein the protein polyurethane alloy has a Young's modulus ranging from about 80 MPa to about 155 MPa.

6. The protein polyurethane alloy of any one of claims 2-5, wherein the polyurethane has a second DMA modulus transition onset temperature in the absence of soy protein, and wherein the second DMA modulus transition onset temperature of the protein polyurethane alloy ranges from about 15° C. to about 100° C. greater than the second DMA modulus transition onset temperature of the polyurethane in the absence of soy protein.

7. The protein polyurethane alloy of any one of claims 2-6, wherein the protein polyurethane alloy has a tensile strength ranging from about 10 MPa to about 18 MPa.

8. The protein polyurethane alloy of any one of claims 2-7, wherein the polyurethane has a moisture vapor transmission rate in the absence of soy protein, and wherein the protein polyurethane alloy has a moisture vapor transmission rate ranging from about 200 g/m²/24 hr to about 300 g/m²/24 hr greater than the moisture vapor transmission rate of the polyurethane in the absence of soy protein.

9. The protein polyurethane alloy of any one of claims 1-8, comprising about 10 wt % to about 50 wt % of the succinylated protein and about 50 wt % to about 90 wt % of the polyurethane.

10. The protein polyurethane alloy of any one of claims 1-8, comprising about 20 wt % to about 35 wt % of the succinylated protein and about 65 wt % to about 80 wt % of the polyurethane.

11. The protein polyurethane alloy of any one of claims 2-10, wherein the protein polyurethane alloy has a color stability defined by a change in a spectrometer measured b* value in the CIELab color space of 6 or less, and wherein the change in the spectrometer measured b* value is a change in b* over six days with the protein polyurethane alloy placed in a hydrolysis chamber at 70° C. and 95% humidity.

12. The protein polyurethane alloy of any one of claims 2-11, wherein the succinylated protein has a lysine modification of about 20% to 100%.

13. The protein polyurethane alloy of any one of claims 2-12, wherein the succinylated protein has a solubility of about 50% to 100%.

14. The protein polyurethane alloy of any one of claims 1-13, further comprising a colored dye.

15. A succinylated soy protein, comprising an average lysine modification of about 20% to 100% and a solubility of about 50% to 100%.

16. The succinylated soy protein of claim 15, wherein the succinylated soy protein is succinylated soy protein isolate.

17. A method of making a protein polyurethane alloy, the method comprising:
succinylating a protein;
blending the succinylated protein with one or more polyurethanes in an aqueous solution to form a blended mixture comprising the succinylated protein dissolved within the one or more polyurethanes; and
removing solvent from the aqueous solution.

18. The method of claim 17, wherein succinylating the protein comprises:
adding succinic anhydride to a protein aqueous solution comprising the protein;
purifying the protein aqueous solution; and
drying the purified protein aqueous solution.

19. The method of claim 17 or claim 18, wherein the protein comprises soy protein.

20. The method of claim 17 or claim 18, wherein the protein comprises soy protein isolate.
